(19) United States
(12) Patent Application Publication
(10) Pub. No.: US 2022/0396817 A1
(43) Pub. Date: Dec. 15, 2022

(54) PRODUCTION OF GLYCOSYLATED PRODUCT IN HOST CELLS

(71) Applicant: INBIOSE N.V., Zwijnaarde (BE)

(72) Inventors: Sofie Aesaert, Zwijnaarde (BE); Joeri Beauprez, Zwijnaarde (BE); Pieter Coussement, Zwijnaarde (BE); Thomas Decoene, Zwijnaarde (BE); Nausicaä Lannoo, Zwijnaarde (BE); Gert Peters, Zwijnaarde (BE); Kristof Vandewalle, Zwijnaarde (BE)

(21) Appl. No.: 17/904,189

(22) PCT Filed: Feb. 12, 2021
(86) PCT No.: PCT/EP2021/053497

§ 371 Date: Aug. 12, 2022

(30) Foreign Application Priority Data
Feb. 14, 2020 (BE) BE2020/5093

(51) Int. Cl. C12P 19/04; C12N 15/90; C12N 9/10; C12N 15/52; C12N 1/20

(52) U.S. Cl. CPC C12P 19/04; C12N 15/902; C12N 9/1096; C12N 9/1048; C12N 15/52; C12N 1/20

(57) ABSTRACT

The present disclosure is in the technical field of synthetic biology and metabolic engineering. The disclosure provides engineered viable bacteria. In particular, the disclosure provides viable bacteria with reduced cell wall biosynthesis additionally modified for production of glycosylated product. The disclosure further provides methods of generating viable bacteria and uses thereof. Furthermore, the disclosure in the technical field of fermentation of metabolically engineered microorganisms producing glycosylated product.

### CROSS-REFERENCE TO RELATED APPLICATIONS

This application is a national phase entry under 35 U.S.C. § 371 of International Patent Application PCT/EP2021/053497, filed Feb. 12, 2021, designating the United States of America and published as International Patent Publication WO 2021/160827 A2 on Aug. 19, 2021, which claims the benefit under Article 8 of the Patent Cooperation Treaty to Belgian Patent Application Serial No. BE2020/5093, filed Feb. 14, 2020.

### STATEMENT ACCORDING TO 37 C.F.R. § 1.821(c) or (e) -SEQUENCE LISTING SUBMITTED AS A TXT AND PDF FILES

Pursuant to 37 C.F.R. § 1.821(c) or (e), files containing a TXT version and a PDF version of the Sequence Listing have been submitted concomitant with this application, the contents of which are hereby incorporated by reference.

### TECHNICAL FIELD

This disclosure is in the technical field of synthetic biology and metabolic engineering. The disclosure provides engineered viable bacteria. In particular, the disclosure provides viable bacteria with reduced cell wall biosynthesis additionally modified for production of glycosylated product. The disclosure further provides methods of generating viable bacteria and uses thereof. Furthermore, the disclosure is in the technical field of fermentation of metabolically engineered microorganisms producing glycosylated product.

### BACKGROUND

The cell wall forms an integral part of the microbial cell. Apart from the first level a cell has with the outside world, it forms a crucial part in the structural integrity of the cell, protecting it against several environmental factors and antimicrobial stresses. The cell wall is mainly built up out of oligo and polysaccharides, forming a structural sugar layer. This layer is synthesized via glycosyltransferases, linking the oligosaccharide moieties together. These glycosyltransferases are also the source for biotechnologists to synthesize glycosylated products, e.g., specialty saccharides (such as disaccharides, oligosaccharide and polysaccharides), glycolipids and glycoproteins as described e.g., in WO2013/087884, WO2012/007481, WO2016/075243 or WO2018/122225. Deletion of the cell wall biosynthetic routes tends to lead to reduced fitness, in particular, on minimal salt media with high osmotic pressure, as shown by Baba et al. 2006, Mol Syst Biol (2006)2:2006.0008. Therefore, to date little to no technologies have attempted to modify the cell wall biosynthesis.

Another problem that occurs during the biochemical synthesis of glycosylated products, is the interference of endogenously present glycosyltransferases with the biosynthesis of complex glycan structures and vice versa, the interference of heterologously introduced glycosyltransferases with the native cell wall biosynthesis routes.

It was further observed that overexpression of certain glycosyltransferases in micro-organisms with specific oligosaccharides or polysaccharides in the cell wall, tend to become slimy and lead to high viscosity production process.

### BRIEF SUMMARY

Summary of the

Surprisingly, it has now been found that the genetically modified microorganisms modified to produce a glycosylated product and with reduced cell wall biosynthesis used in the disclosure provide for newly identified microorganisms having a similar or positive effect on fermentative production of glycosylated product, in terms of yield, productivity, specific productivity and/or growth speed. In the production of glycosylated products such as oligosaccharides, little to no effect was observed on the fitness, as exemplified with the growth rate. Moreover, these modifications may improve some of the production parameters, such as viscosity, airlift and foaming. These parameters impact the mass transfer of a bioreactor (e.g., the oxygen transfer) and the vessel filling of a bioreactor, i.e., increasing the amount of product per total bioreactor volume.

Provided herein are tools and methods by means of which glycosylated products can be produced in an efficient, time and cost-effective way and which yield high amounts of the desired product.

Further provided herein are a cell and a method for the production of a glycosylated product wherein the cell is genetically modified for the production of the glycosylated product and comprises a reduced cell wall biosynthesis.

### Definitions

The words used in this specification to describe the disclosure and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various embodiments and aspects of embodiments of the disclosure disclosed herein are to be understood not only in the order and context specifically described in this specification, but to include any order and any combination thereof. Whenever the context requires, all words used in the singular number shall be deemed to include the plural and vice versa. Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the specification, there have been disclosed embodiments of the disclosure, and although specific terms are employed, the terms are used in a descriptive sense only and not for purposes of limitation, the scope of the disclosure being set forth in the following claims. It must be understood that the illustrated embodiments have been set forth only for the purposes of example and that it should not be taken as limiting the disclosure. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims that follow, reference characters used to designate claim steps are provided for convenience of description only, and are not intended to imply any particular order for performing the steps.

According to the disclosure, the term “polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” according to the disclosure. Moreover, DNAs or RNAs comprising unusual bases, such as inosine, or modified bases, such as tritylated bases, are to be understood to be covered by the term “polynucleotides.” It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid sidechains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, gamma-carboxylation of glutamic acid residues, hydroxylation and ADP-ribosylation, selenoylation, transfer-RNA mediated addition of amino acids to proteins, such as arginylation, and ubiquitination. Polypeptides may be branched or cyclic, with or without branching. Cyclic, branched and branched circular polypeptides may result from post-translational natural processes and may be made by entirely synthetic methods, as well.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated,” as the term is employed herein. Similarly, a “synthetic” sequence, as the term is used herein, means any sequence that has been generated synthetically and not directly isolated from a natural source. “Synthesized,” as the term is used herein, means any synthetically generated sequence and not directly isolated from a natural source.

“Recombinant” means genetically engineered DNA prepared by transplanting or splicing genes from one species into the cells of a host organism of a different species. Such DNA becomes part of the host's genetic makeup and is replicated. “Mutant” cell or microorganism as used within the context of the disclosure refers to a cell or microorganism, which is genetically engineered or has an altered genetic make-up.

The term “endogenous,” within the context of the disclosure refers to any polynucleotide, polypeptide or protein sequence that is a natural part of a cell and is occurring at its natural location in the cell chromosome. The term “exogenous” refers to any polynucleotide, polypeptide or protein sequence that originates from outside the cell under study and not a natural part of the cell or which is not occurring at its natural location in the cell chromosome or plasmid.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism species. In contrast a “homologous” polynucleotide, gene, nucleic acid, polypeptide, or enzyme is used herein to denote a polynucleotide, gene, nucleic acid, polypeptide, or enzyme that is derived from the host organism species. When referring to a gene regulatory sequence or to an auxiliary nucleic acid sequence used for maintaining or manipulating a gene sequence (e.g., a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e., in the genome of a non-genetically engineered organism) is referred to herein as a “heterologous promoter,” even though the promoter may be derived from the same species (or, in some cases, the same organism) as the gene to which it is linked.

The term “polynucleotide encoding a polypeptide” as used herein encompasses polynucleotides that include a sequence encoding a polypeptide of the disclosure. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

The term “modified expression” of a gene relates to a change in expression compared to the wild type expression of the gene in any phase of biosynthesis of the product. The modified expression is either a lower or higher expression compared to the wild type, wherein the term “higher expression” is also defined as “overexpression” of the gene in the case of an endogenous gene or “expression” in the case of a heterologous gene that is not present in the wild type strain. Lower expression or reduced expression is obtained by means of common well-known technologies for a skilled person (such as the usage of siRNA, CRISPR, CRISPRi, riboswitch, recombineering, homologous recombination, ssDNA mutagenesis, RNAi, miRNA, asRNA, mutating genes, knocking-out genes, transposon mutagenesis, etc.), which are used to change the genes in such a way that they are less-able (i.e., statistically significantly ‘less-able’ compared to a functional wild-type gene) or completely unable (such as knocked-out genes) to produce functional final products. Lower expression or reduced expression can, for instance, be obtained by mutating one or more base pairs in the promoter sequence or changing the promoter sequence fully to a constitutive promoter with a lower expression strength compared to the wild type or an inducible promoter, which result in regulated expression or a repressible promoter, which results in regulated expression. Overexpression or expression is obtained by means of common well-known technologies for a skilled person, wherein the gene is part of an “expression cassette,” which relates to any sequence in which a promoter sequence, untranslated region sequence (UTR) (containing either a ribosome binding sequence or Kozak sequence), a coding sequence (for instance, a membrane protein gene sequence) and optionally a transcription terminator is present, and leading to the expression of a functional active protein. The expression is either constitutive or conditional or regulated.

The term “riboswitch” as used herein is defined to be part of the messenger RNA that folds into intricate structures that block expression by interfering with translation. Binding of an effector molecule induces conformational change(s) permitting regulated expression post-transcriptionally.

The term “constitutive expression” is defined as expression that is not regulated by transcription factors other than the subunits of RNA polymerase (e.g., the bacterial sigma factors) under certain growth conditions. Non-limiting examples of such transcription factors are CRP, LacI, ArcA, Cra, IclR in E coli, or, Aft2p, Crzlp, Skn7 in Saccharomyces cerevisiae, or, DeoR, GntR, Fur in B. subtilis. These transcription factors bind on a specific sequence and may block or enhance expression in certain growth conditions. RNA polymerase binds a specific sequence to initiate transcription, for instance, via a sigma factor in prokaryotic hosts.

The term “regulated expression” is defined as expression that is regulated by transcription factors other than the subunits of RNA polymerase (e.g., bacterial sigma factors) under certain growth conditions. Examples of such transcription factors are described above. Commonly expression regulation is obtained by means of an inducer, such as but not limited to IPTG, arabinose, rhamnose, fucose, allo-lactose or pH shifts, or temperature shifts or carbon depletion or substrates or the produced product.

The term “wild type” refers to the commonly known genetic or phenotypical situation as it occurs in nature.

The term “glycosylated product” as used herein refers to the group of molecules comprising at least one monosaccharide as defined herein. Examples of such glycosylated products include, but are not limited to, monosaccharide, phosphorylated monosaccharide, activated monosaccharide, disaccharide, oligosaccharide, glycoprotein, nucleoside, glycosylphosphate, glycoprotein and glycolipid.

The term “monosaccharide” as used herein refers to saccharides containing only one simple sugar. Examples of monosaccharides comprise Hexose, D-Glucopyranose, D-Galactofuranose, D-Galactopyranose, L-Galactopyranose, D-Mannopyranose, D-Allopyranose, L-Altropyranose, D-Gulopyranose, L-Idopyranose, D-Talopyranose, D-Ribofuranose, D-Ribopyranose, D-Arabinofuranose, D-Arabinopyranose, L-Arabinofuranose, L-Arabinopyranose, D-Xylopyranose, D-Lyxopyranose, D-Erythrofuranose, D-Threofuranose, Heptose, L-glycero-D-manno-Heptopyranose (LDmanHep), D-glycero-D-manno-Heptopyranose (DDmanHep), 6-Deoxy-L-altropyranose, 6-Deoxy-D-gulopyranose, 6-Deoxy-D-talopyranose, 6-Deoxy-D-galactopyranose, 6-Deoxy-L-galactopyranose, 6-Deoxy-D-mannopyranose, 6-Deoxy-L-mannopyranose, 6-Deoxy-D-glucopyranose, 2-Deoxy-D-arabino-hexose, 2-Deoxy-D-erythro-pentose, 2,6-Dideoxy-D-arabino-hexopyranose, 3,6-Dideoxy-D-arabino-hexopyranose, 3,6-Dideoxy-L-arabino-hexopyranose, 3,6-Dideoxy-D-xylo-hexopyranose, 3,6-Dideoxy-D-ribo-hexopyranose, 2,6-Dideoxy-D-ribo-hexopyranose, 3,6-Dideoxy-L-xylo-hexopyranose, 2-Amino-2-deoxy-D-glucopyranose, 2-Amino-2-deoxy-D-galactopyranose, 2-Amino-2-deoxy-D-mannopyranose, 2-Amino-2-deoxy-D-allopyranose, 2-Amino-2-deoxy-L-altropyranose, 2-Amino-2-deoxy-D-gulopyranose, 2-Amino-2-deoxy-L-idopyranose, 2-Amino-2-deoxy-D-talopyranose, 2-Acetamido-2-deoxy-D-glucopyranose, 2-Acetamido-2-deoxy-D-galactopyranose, 2-Acetamido-2-deoxy-D-mannopyranose, 2-Acetamido-2-deoxy-D-allopyranose, 2-Acetamido-2-deoxy-L-altropyranose, 2-Acetamido-2-deoxy-D-gulopyranose, 2-Acetamido-2-deoxy-L-idopyranose, 2-Acetamido-2-deoxy-D-talopyranose, 2-Acetamido-2,6-dideoxy-D-galactopyranose, 2-Acetamido-2,6-dideoxy-L-galactopyranose, 2-Acetamido-2,6-dideoxy-L-mannopyranose, 2-Acetamido-2,6-dideoxy-D-glucopyranose, 2-Acetamido-2,6-dideoxy-L-altropyranose, 2-Acetamido-2,6-dideoxy-D-talopyranose, D-Glucopyranuronic acid, D-Galactopyranuronic acid, D-Mannopyranuronic acid, D-Allopyranuronic acid, L-Altropyranuronic acid, D-Gulopyranuronic acid, L-Gulopyranuronic acid, L-Idopyranuronic acid, D-Talopyranuronic acid, Sialic acid, 5-Amino-3,5-dideoxy-D-glycero-D-galacto-non-2-ulosonic acid, 5-Acetamido-3,5-dideoxy-D-glycero-D-galacto-non-2-ulosonic acid, 5-Glycolylamido-3,5-dideoxy-D-glycero-D-galacto-non-2-ulosonic acid, Erythritol, Arabinitol, Xylitol, Ribitol, Glucitol, Galactitol, Mannitol, D-ribo-Hex-2-ulopyranose, D-arabino-Hex-2-ulofuranose (D-fructofuranose), D-arabino-Hex-2-ulopyranose, L-xylo-Hex-2-ulopyranose, D-lyxo-Hex-2-ulopyranose, D-threo-Pent-2-ulopyranose, D-altro-Hept-2-ulopyranose, 3-C-(Hydroxymethyl)-D-erythofuranose, 2,4,6-Trideoxy-2,4-diamino-D-glucopyranose, 6-Deoxy-3-O-methyl-D-glucose, 3-O-Methyl-D-rhamnose, 2,6-Dideoxy-3-methyl-D-ribo-hexose, 2-Amino-3-O-[(R)-1-carboxyethyl]-2-deoxy-D-glucopyranose, 2-Acetamido-3-O-[(R)-carboxyethyl]-2-deoxy-D-glucopyranose, 2-Glycolylamido-3-O-[(R)-1-carboxyethyl]-2-deoxy-D-glucopyranose, 3-Deoxy-D-lyxo-hept-2-ulopyranosaric acid, 3-Deoxy-D-manno-oct-2-ulopyranosonic acid, 3-Deoxy-D-glycero-D-galacto-non-2-ulopyranosonic acid, 5,7-Diamino-3,5,7,9-tetradeoxy-L-glycero-L-manno-non-2-ulopyranosonic acid, 5,7-Diamino-3,5,7,9-tetradeoxy-L-glycero-L-altro-non-2-ulopyranosonic acid, 5,7-Diamino-3,5,7,9-tetradeoxy-D-glycero-D-galacto-non-2-ulopyranosonic acid, 5,7-Diamino-3,5,7,9-tetradeoxy-D-glycero-D-talo-non-2-ulopyranosonic acid, glucose, galactose, N-acetylglucosamine, glucosamine, mannose, xylose, N-acetylmannosamine, N-acetylneuraminic acid, N-glycolylneuraminic acid, a sialic acid, N-acetylgalactosamine, galactosamine, fucose, rhamnose, glucuronic acid, gluconic acid, fructose and polyols.

The term “phosphorylated monosaccharide” as used herein refers to one of the above listed monosaccharides, which is phosphorylated. Examples of phosphorylated monosaccharides include but are not limited to glucose-1-phosphate, glucose-6-phosphate, glucose-1,6-bisophosphate, galactose-1-phosphate, fructose-6-phosphate, fructose-1,6-bisphosphate, fructose-1-phosphate, glucosamine-1-phosphate, glucosamine-6-phosphate, N-acetylglucosamine-1-phosphate, mannose-1-phosphate, mannose-6-phosphate or fucose-1-phosphate. Some, but not all, of these phosphorylated monosaccharides are precursors or intermediates for the production of activated monosaccharide.

The terms “activated monosaccharide,” “nucleotide-activated sugar,” “nucleotide-sugar,” “activated sugar,” “nucleoside” or “nucleotide donor” as used herein can be used interchangeably and refer to activated forms of monosaccharides, such as the monosaccharides as listed here above. Examples of activated monosaccharides include but are not limited to GDP-fucose, GDP-mannose, CMP-N-acetylneuraminic acid, CMP-N-glycolylneuraminic acid, UDP-glucuronate, UDP-N-acetylgalactosamine, UDP-glucose, UDP-galactose, CMP-sialic acid and UDP-N-acetylglucosamine. Activated monosaccharides, also known as nucleotide sugars, act as glycosyl donors in glycosylation reactions. Those reactions are catalyzed by a group of enzymes called glycosyltransferases.

The term “glycosyltransferase” as used herein refers to an enzyme capable to catalyze the transfer of sugar moieties from activated donor molecules to specific acceptor molecules, forming glycosidic bonds. A classification of glycosyltransferases using nucleotide diphospho-sugar, nucleotide monophospho-sugar and sugar phosphates and related proteins into distinct sequence-based families has been described (Campbell et al., Biochem. J. 326, 929-939 (1997)) and is available on the CAZy (CArbohydrate-Active EnZymes) website (www.cazy.org).

Fucosyltransferases are glycosyltransferases that transfer a fucose residue (Fuc) from a GDP-fucose (GDP-Fuc) donor onto a glycan acceptor. Fucosyltransferases comprise alpha-1,2-fucosyltransferases, alpha-1,3-fucosyltransferases, alpha-1,4-fucosyltransferases and alpha-1,6-fucosyltransferases that catalyze the transfer of a Fuc residue from GDP-Fuc onto a glycan acceptor via alpha-glycosidic bonds. Fucosyltransferases can be found but are not limited to the GT10, GT11, GT23, GT65 and GT68 CAZy families. Sialyltransferases are glycosyltransferases that transfer a sialyl group (like Neu5Ac or Neu5Gc) from a donor (like CMP-Neu5Ac or CMP-Neu5Gc) onto a glycan acceptor. Sialyltransferases comprise alpha-2,3-sialyltransferases and alpha-2,6-sialyltransferases that catalyze the transfer of a sialyl group onto a glycan acceptor via alpha-glycosidic bonds. Sialyltransferases can be found but are not limited to the GT29, GT42, GT80 and GT97 CAZy families. Galactosyltransferases are glycosyltransferases that transfer a galactosyl group (Gal) from an UDP-galactose (UDP-Gal) donor onto a glycan acceptor.

Galactosyltransferases comprise beta-1,3-galactosyltransferases, beta-1,4-galactosyltransferases, alpha-1,3-galactosyltransferases and alpha-1,4-galactosyltransferases that transfer a Gal residue from UDP-Gal onto a glycan acceptor via alpha- or beta-glycosidic bonds. Galactosyltransferases can be found but are not limited to the GT2, GT6, GT8, GT25 and GT92 CAZy families. Glucosyltransferases are glycosyltransferases that transfer a glucosyl group (Glc) from an UDP-glucose (UDP-Glc) donor onto a glycan acceptor. Glucosyltransferases comprise alpha-glucosyltransferases, beta-1,2-glucosyltransferases, beta-1,3-glucosyltransferases and beta-1,4-glucosyltransferases that transfer a Glc residue from UDP-Glc onto a glycan acceptor via alpha- or beta-glycosidic bonds. Glucosyltransferases can be found but are not limited to the GT1, GT4 and GT25 CAZy families. Mannosyltransferases are glycosyltransferases that transfer a mannose group (Man) from a GDP-mannose (GDP-Man) donor onto a glycan acceptor. Mannosyltransferases comprise alpha-1,2-mannosyltransferases, alpha-1,3-mannosyltransferases and alpha-1,6-mannosyltransferases that transfer a Man residue from GDP-Man onto a glycan acceptor via alpha-glycosidic bonds. Mannosyltransferases can be found but are not limited to the GT22, GT39, GT62 and GT69 CAZy families. N-acetylglucosaminyltransferases are glycosyltransferases that transfer an N-acetylglucosamine group (GlcNAc) from an UDP-N-acetylglucosamine (UDP-GlcNAc) donor onto a glycan acceptor. N-acetylglucosaminyltransferases can be found but are not limited to GT2 and GT4 CAZy families. N-acetylgalactosaminyltransferases are glycosyltransferases that transfer an N-acetylgalactosamine group (GalNAc) from an UDP-N-acetylgalactosamine (UDP-GalNAc) donor onto a glycan acceptor. N-acetylgalactosaminyltransferases can be found but are not limited to GT7, GT12 and GT27 CAZy families. N-acetylmannosaminyltransferases are glycosyltransferases that transfer an N-acetylmannosamine group (ManNAc) from an UDP-N-acetylmannosamine (UDP-ManNAc) donor onto a glycan acceptor. Xylosyltransferases are glycosyltransferases that transfer a xylose residue (Xyl) from an UDP-xylose (UDP-Xyl) donor onto a glycan acceptor. Xylosyltransferases can be found but are not limited to GT14, GT61 and GT77 CAZy families. Glucuronyltransferases are glycosyltransferases that transfer a glucuronate from an UDP-glucuronate donor onto a glycan acceptor via alpha- or beta-glycosidic bonds. Glucuronyltransferases can be found but are not limited to GT4, GT43 and GT93 CAZy families. Galacturonyltransferases are glycosyltransferases that transfer a galacturonate from an UDP-galacturonate donor onto a glycan acceptor. N-glycolylneuraminyltransferases are glycosyltransferases that transfer an N-glycolylneuraminic acid group (Neu5Gc) from a CMP-Neu5Gc donor onto a glycan acceptor. Rhamnosyltransferases are glycosyltransferases that transfer a rhamnose residue from a GDP-rhamnose donor onto a glycan acceptor. Rhamnosyltransferases can be found but are not limited to the GT1, GT2 and GT102 CAZy families. N-acetylrhamnosyltransferases are glycosyltransferases that transfer an N-acetylrhamnosamine residue from an UDP-N-acetyl-L-rhamnosamine donor onto a glycan acceptor. UDP-4-amino-4,6-dideoxy-N-acetyl-beta-L-altrosamine transaminases are glycosyltransferases that use an UDP-2-acetamido-2,6-dideoxy-L-arabino-4-hexulose in the biosynthesis of pseudaminic acid, which is a sialic acid-like sugar that is used to modify flagellin. Fucosaminyltransferases are glycosyltransferases that transfer an N-acetylfucosamine residue from a dTDP-N-acetylfucosamine or an UDP-N-acetylfucosamine donor onto a glycan acceptor.

The term “galactoside beta-1,3-N-acetylglucosaminyltransferase” refers to a glycosyltransferase that is capable to transfer an N-acetylglucosamine (GlcNAc) residue from UDP-GlcNAc to the terminal galactose residue of lactose in a beta-1,3 linkage.

The term “disaccharide” as used herein refers to a saccharide polymer containing two simple sugars, i.e., monosaccharides. Such disaccharides contain monosaccharides selected from the list as used herein above. Examples of disaccharides comprise, but are not limited to, lactose, N-acetyllactosamine, Lacto-N-biose, lactulose, sucrose, maltose, trehalose.

“Oligosaccharide” as the term is used herein and as generally understood in the state of the art, refers to a saccharide polymer containing a small number, typically three to fifteen, of simple sugars, i.e., monosaccharides. Preferably the oligosaccharide as described herein contains monosaccharides selected from the list as used herein above. Examples of oligosaccharides include but are not limited to Lewis-type antigen oligosaccharides, neutral oligosaccharides, fucosylated oligosaccharides, sialylated oligosaccharides, and mammalian milk oligosaccharides.

As used herein, “mammalian milk oligosaccharide” refers to oligosaccharides such as but not limited to 3-fucosyllactose, 2′-fucosyllactose, 6-fucosyllactose, 2′,3-difucosyllactose, 2′,2-difucosyllactose, 3,4-dificosyllactose, 6′-sialyllactose, 3′-sialyllactose, 3,6-disialyllactose, 6,6′-disialyllactose, 3,6-disialyllacto-N-tetraose, lactodifucotetraose, lacto-N-tetraose, lacto-N-neotetraose, lacto-N-fucopentaose IL, lacto-N-fucopentaose I, lacto-N-fucopentaose III, lacto-N-fucopentaose V, lacto-N-fucopentaose VI, sialyllacto-N-tetraose d (LSTd), sialyllacto-N-tetraose c (LSTc), sialyllacto-N-tetraose b (LSTb), sialyllacto-N-tetraose a (LSTa), lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated milk oligosaccharides, neutral milk oligosaccharide and/or sialylated milk oligosaccharides.

As used herein the term “Lewis-type antigens” comprise the following oligosaccharides: H1 antigen, which is Fucα1-2Galβ1-3GlcNAc, or in short 2′FLNB; Lewis^{a}, which is the trisaccharide Galβ1-3[Fucα1-4]GlcNAc, or in short 4-FLNB; Lewis^{b}, which is the tetrasaccharide Fucα1-2Galβ1-3[Fucα1-4]GcNAc, or in short DiF-LNB; sialyl Lewis^{a}, which is 5-acetylneuraminyl-(2-3)-galactosyl-(1-3)-(fucopyranosyl-(1-4))-N-acetylglucosamine, or written in short Neu5Acα2-3Galβ1-3[Fucα1-4]GlcNAc; H2 antigen, which is Fucα1-2Galβ1-4GlcNAc, or otherwise stated 2′fucosyl-N-acetyl-lactosamine, in short 2′FLacNAc; Lewis^{x}, which is the trisaccharide Galβ1-4[Fucα1-3]GlcNAc, or otherwise known as 3-Fucosyl-N-acetyl-lactosamine, in short 3-FLacNAc, Lewis^{y}, which is the tetrasaccharide Fucα1-2Galβ1-4[Fucα1-3]GcNAc and sialyl Lewis^{x}, which is 5-acetylneuraminyl-(2-3)-galactosyl-(1-4)-(fucopyranosyl-(1-3))-N-acetylglucosamine, or written in short Neu5Acα2-3Galβ1-4[Fucα1-3]GcNAc.

The term “sialylated oligosaccharide” as used herein refers to a sugar polymer containing at least two monosaccharide units, at least one of which is a sialyl (N-acetylneuraminyl) moiety. The sialylated oligosaccharide can have a linear or branched structure containing monosaccharide units that are linked to each other by interglycosidic linkage.

As used herein, a “sialylated oligosaccharide” is furthermore to be understood as a charged sialic acid containing oligosaccharide, i.e., an oligosaccharide having a sialic acid residue. It has an acidic nature. Some examples are 3-SL (3′-sialyllactose), 3′-sialyllactosamine, 6-SL (6′-sialyllactose), 6′-sialyllactosamine, oligosaccharides comprising 6′-sialyllactose, SGG hexasaccharide (Neu5Acα-2,3Gal beta-1,3GalNac beta-1,3Gala-1,4Gal beta-1,4Gal), sialylated tetrasaccharide (Neu5Acα-2,3Gal beta-1,4GlcNac beta-14GlcNAc), pentasaccharide LSTD (Neu5Acα-2,3Gal beta-1,4GlcNac beta-1,3Gal beta-1,4Glc), sialylated lacto-N-triose, sialylated lacto-N-tetraose, sialyllacto-N-neotetraose, monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, 3′-sialyl-3-fucosyllactose, disialomonofucosyllacto-N-neohexaose, monofucosylmonosialyllacto-N-octaose (sialyl Lea), sialyllacto-N-fucohexaose IL, disialyllacto-N-fucopentaose IL, monofucosyldisialyllacto-N-tetraose and oligosaccharides bearing one or several sialic acid residue(s), including but not limited to: oligosaccharide moieties of the gangliosides selected from GM3 (3′sialyllactose, Neu5Acα-2,3Gal β-4Glc) and oligosaccharides comprising the GM3 motif, GD3 (Neu5Acα-2,8Neu5Acα-2,3Gal β-1,4Glc), GT3 (Neu5Acα-2,8Neu5Acα-2,8Neu5Acα-2,3Gal β-1,4Glc), GM2 (GalNAc β-1,4(Neu5Acα-2,3)Gal β-1,4Glc), GM1 (Gal β-1,3GalNAc β-1,4(Neu5Acα-2,3)Gal β-1,4Glc), GD1a (Neu5Acα-2,3Gal β-1,3GalNAc β-1,4(Neu5Acα-2,3)Gal β-1,4Glc), GT1a (Neu5Acα-2,8Neu5Acα-2,3Gal β-1,3GalNAc β-1,4(Neu5Acα-2,3)Gal β-1,4Glc), GD2 (GalNAc β-1,4(Neu5Acα-2,8Neu5Acα2,3)Gal β-1,4Glc), GT2 (GalNAc β-1,4(Neu5Acα-2,8Neu5Acα-2,8Neu5Acα2,3)Gal β-1,4Glc), GD1b (Gal β-1,3GalNAc β-1,4(Neu5Acα-2,8Neu5Acα2,3)Gal β-1,4Glc), GT1b (Neu5Acα-2,3Gal β-1,3GalNAc β-1,4(Neu5Acα-2,8Neu5Acα2,3)Gal β-1,4Glc), GQ1b (Neu5Acα-2,8Neu5Acα-2,3Gal β-1,3GalNAc β-1,4(Neu5Acα-2,8Neu5Acα2,3)Gal β-1,4Glc), GT1c (Gal β-1,3GalNAc β-1,4(Neu5Acα-2,8Neu5Acα-2,8Neu5Acα2,3)Gal β-1,4Glc), GQ1c (Neu5Acα-2,3Gal β-1,3GalNAc β-1,4(Neu5Acα-2,8Neu5Acα-2,8Neu5Acα2,3)Gal β-1,4Glc), GP1c (Neu5Acα-2,8Neu5Acα-2,3Gal β-1,3GalNAc β-1,4(Neu5Acα-2,8Neu5Acα-2,8Neu5Acα2,3)Gal β-1,4Glc), GD1a (Neu5Acα-2,3Gal β-1,3(Neu5Acα-2,6)GalNAc β-1,4Gal β-1,4Glc), Fucosyl-GM1 (Fuca-1,2Gal β-1,3GalNAc β-1,4(Neu5Acα-2,3)Gal β-1,4Glc); all of which may be extended to the production of the corresponding gangliosides by reacting the above oligosaccharide moieties with ceramide or synthetizing the above oligosaccharides on a ceramide.

Preferably the sialylated oligosaccharide is a sialylated mammalian milk oligosaccharide, also known as acidic mammalian milk oligosaccharides. Examples of acidic mammalian milk oligosaccharides include, but are not limited to, 3′-sialyllactose (3′-O-sialyllactose, 3′-SL, 3′SL), 6′-sialyllactose (6′-O-sialyllactose, 6′-SL, 6′SL), 3-fucosyl-3′-sialyllactose (3′-O-sialyl-3-O-fucosyllactose, FSL), 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-LNH (SLNH), sialyl-lacto-N-hexaose (SLNH), sialyl-lacto-N-neohexaose I (SLNH-I), sialyl-lacto-N-neohexaose II (SLNH-II), disialyl-lacto-N-tetraose (DS-LNT), 6′-O-sialylated-lacto-N-neotetraose, 3′-O-sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose. In some sialylated mammalian milk oligosaccharides the sialic acid residue is preferably linked to the 3-O— and/or 6-O— position of a terminal D-galactose or to the 6-O— position of a non-terminal GlcNAc residue via α-glycosidic linkages.

A “fucosylated oligosaccharide” as used herein and as generally understood in the state of the art is an oligosaccharide that is carrying a fucose-residue. Examples comprise 2′-fucosyllactose, 3-fucosyllactose, 4 fucosyllactose, 6 fucosyllactose, difucosyllactose, lactodifucotetraose (LDFT), Lacto-N-fucopentaose I (LNF I), Lacto-N-fucopentaose II (LNF-II), Lacto-N-fucopentaose III (LNF III), lacto-N-fucopentaose V (LNF V), lacto-N-fucopentaose VI (LNF VI), lacto-N-neofucopentaose I, lacto-N-difucohexaose I (LDFH I), lacto-N-difucohexaose II (LDFH II), Monofucosyllacto-N-hexaose III (MFLNH III), Difucosyllacto-N-hexaose (DFLNHa), difucosyl-lacto-N-neohexaose. Preferably the fucosylated oligosaccharide is a fucosylated mammalian milk oligosaccharide, also known as fucosylated mammalian milk oligosaccharides.

A “neutral oligosaccharide” as used herein and as generally understood in the state of the art is an oligosaccharide that has no negative charge originating from a carboxylic acid group. Examples of such neutral oligosaccharide are 2′-fucosyllactose, 3-fucosyllactose, 2′, 3-difucosyllactose, lacto-N-triose II, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, 6′-galactosyllactose, 3′-galactosyllactose, lacto-N-hexaose, lacto-N-neohexaose, para-lacto-N-hexaose, para-lacto-N-neohexaose, difucosyl-lacto-N-hexaose and difucosyl-lacto-N-neohexaose. Preferably the neutral oligosaccharide is a neutral mammalian milk oligosaccharide, also known as neutral mammalian milk oligosaccharides.

As used herein, the term “glycolipid” refers to any of the glycolipids, which are generally known in the art. Glycolipids (GLs) can be subclassified into Simple (SGLs) and Complex (CGLs) glycolipids. Simple GLs, sometimes called saccharolipids, are two-component (glycosyl and lipid moieties) GLs in which the glycosyl and lipid moieties are directly linked to each other. Examples of SGLs include glycosylated fatty acids, fatty alcohols, carotenoids, hopanoids, sterols or paraconic acids. Bacterially produced SGLs can be classified into rhamnolipids, glucolipids, trehalolipids, other glycosylated (non-trehalose containing) mycolates, trehalose-containing oligosaccharide lipids, glycosylated fatty alcohols, glycosylated macro-lactones and macro-lactams, glycomacrodiolides (glycosylated macrocyclic dilactones), glyco-carotenoids and glyco-terpenoids, and glycosylated hopanoids/sterols. Complex glycolipids (CGLs) are, however, structurally more heterogeneous, as they contain, in addition to the glycosyl and lipid moieties, other residues like, for example, glycerol (glycoglycerolipids), peptide (glycopeptidolipids), acylated-sphingosine (glycosphingolipids), or other residues (lipopolysaccharides, phenolic glycolipids, nucleoside lipids).

The term polyol as used herein is an alcohol containing multiple hydroxyl groups. For example, glycerol, sorbitol, or mannitol.

The term “sialic acid” as used herein refers to the group comprising sialic acid, neuraminic acid, N-acetylneuraminic acid and N-glycolylneuraminic acid.

The terms “cell genetically modified for the production of glycosylated product” within the context of the disclosure refers to a cell of a microorganism, which is genetically manipulated to comprise at least one of i) a gene encoding a glycosyltransferase necessary for the synthesis of the glycosylated, ii) a biosynthetic pathway to produce a nucleotide donor suitable to be transferred by the glycosyltransferase to a carbohydrate precursor, and/or iii) a biosynthetic pathway to produce a precursor or a mechanism of internalization of a precursor from the culture medium into the cell where it is glycosylated to produce the glycosylated product.

The terms “nucleic acid sequence coding for an enzyme for glycosylated product synthesis” relates to nucleic acid sequences coding for enzymes necessary in the synthesis pathway to the glycosylated product. The synthesis pathway to the glycosylated product comprise but are not limited to a fucosylation, sialylation, galactosylation, N-acetylglucosaminylation, N-acetylgalactosylation, mannosylation, N-acetylmannosinylation pathway.

Examples of such enzymes useful in the synthesis pathway to the glycosylated product are fructose-6-P-aminotransferases (e.g., glmS), glucosamine-6-P-aminotransferases (e.g., a heterologous GNA1), (native) phosphatases, N-acetylglucosamine-2-epimerases (e.g., a heterologous AGE), sialic acid synthases (e.g., a heterologous neuB), CMP-sialic acid synthetases (e.g., a heterologous neuA), UDP-N-acetylglucosamine-2-epimerases, ManNAc kinase forming ManNAc-6P, sialic acid phosphate synthetase forming Neu5Ac-9P, sialic acid phosphatase forming sialic acid, sialyltransferases, alfa-2,3-sialyltransferase, alfa-2,6-sialyltransferase, alfa-2,8-sialyltransferase.

A “fucosylation pathway” as used herein is a biochemical pathway consisting of the enzymes and their respective genes, mannose-6-phosphate isomerase, phosphomannomutase, mannose-1-phosphate guanylyltransferase, GDP-mannose 4,6-dehydratase, GDP-L-fucose synthase and/or the salvage pathway L-fucokinase/GDP-fucose pyrophosphorylase, combined with a fucosyltransferase leading to α 1,2; α 1,3; α 1,4 or α 1,6 fucosylated oligosaccharides.

A “sialylation pathway” is a biochemical pathway consisting of the enzymes and their respective genes, L-glutamine-D-fructose-6-phosphate aminotransferase, glucosamine-6-phosphate deaminase, phosphoglucosamine mutase, N-acetylglucosamine-6-phosphate deacetylase, N-acetylglucosamine epimerase, UDP-N-acetylglucosamine 2-epimerase, N-acetylglucosamine-6P 2-epimerase, Glucosamine 6-phosphate N-acetyltransferase, N-AcetylGlucosamine-6-phosphate phosphatase, N-acetylmannosamine-6-phosphate phosphatase, N-acetylmannosamine kinase, phosphoacetylglucosamine mutase, N-acetylglucosamine-1-phosphate uridyltransferase, glucosamine-1-phosphate acetyltransferase, sialic acid synthase, N-acetylneuraminate lyase, N-acylneuraminate-9-phosphate synthase, N-acylneuraminate-9-phosphate phosphatase, and/or CMP-sialic acid synthase, combined with a sialyltransferase leading to α 2,3; α 2,6 α 2,8 sialylated oligosaccharides.

A “galactosylation pathway” as used herein is a biochemical pathway consisting of the enzymes and their respective genes, galactose-1-epimerase, galactokinase, glucokinase, galactose-1-phosphate uridylyltransferase, UDP-glucose 4-epimerase, glucose-1-phosphate uridylyltransferase, and/or glucophosphomutase, combined with a galactosyltransferase leading to an alpha or beta bound galactose on the 2, 3, 4, 6 hydroxyl group of a mono-, di-, or oligosaccharide.

An “N-acetylglucosaminylation pathway” as used herein is a biochemical pathway consisting of the enzymes and their respective genes, L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridyltransferase, glucosamine-1-phosphate acetyltransferase, and/or glucosamine-1-phosphate acetyltransferase, combined with a glycosyltransferase leading to an alpha or beta bound N-acetylglucosamine on the 3, 4, 6 hydroxylgroup of a mono-, di- or oligosaccharide.

An “N-acetylgalactosylation pathway” as used herein is a biochemical pathway consisting of the enzymes and their respective genes, L-glutamine-D-fructose-6-phosphate aminotransferase, phosphoglucosamine mutase, N-acetylglucosamine 1-phosphate uridylyltransferase, UDP-N-acetylglucosamine 4-epimerase, UDP-galactose 4-epimerase, N-acetylgalactosamine kinase and/or UDP-GalNAc pyrophosphorylase combined with a glycosyltransferase leading to an alpha or beta bound N-acetylgalactosamine on a mono-, di- or oligosaccharide.

A “mannosylation pathway” as used herein is a biochemical pathway consisting of the enzymes and their respective genes, mannose-6-phosphate isomerase, phosphomannomutase and/or mannose-1-phosphate guanyltransferase combined with a glycosyltransferase leading to an alpha or beta bound mannose on a mono-, di- or oligosaccharide.

An “N-acetylmannosinylation pathway” as used herein is a biochemical pathway consisting of the enzymes and their respective genes, L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridyltransferase, glucosamine-1-phosphate acetyltransferase, glucosamine-1-phosphate acetyltransferase, UDP-GlcNAc 2-epimerase and/or ManNAc kinase combined with a glycosyltransferase leading to an alpha or beta bound N-acetylmannosamine on a mono-, di- or oligosaccharide.

The term “cell wall biosynthesis pathway” as used herein is a biochemical pathway consisting of the enzymes and their respective genes involved in the synthesis of components of the cell wall. Components of the cell wall comprise oligosaccharides comprising D- or L-glucose, D- or L-galactose, mannose, N-acetylglucosamine, N-acetylmannosamine, N-acetylgalactosamine, L-fucose, N-acetylneuraminic acid, L-rhamnose (Herget et al., 2008, BMC Struct. Biol. 8:35, doi:10.1186/1472-6807-8-35).

The term “cell wall carbohydrate antigen biosynthesis” as used herein is a biochemical pathway consisting of the enzymes and their respective genes involved in the synthesis of cell wall carbohydrate antigen.

The term “cell wall carbohydrate antigen” refers to a carbohydrate chain linked to a protein or a lipid residing in the cell wall wherein the carbohydrate chain elicits an immune response. The term “O-antigen biosynthesis gene cluster” as used herein refers to a group of genes that encode enzymes that are involved in the biosynthesis of the O-antigen. The O-antigen biosynthesis gene cluster comprises genes involved in nucleotide sugar biosynthesis, glycosyltransferases and O-antigen processing genes (Samuel and Reeves, 2003, Carbohydr. Res. 338:23, 2503-2519).

The term “common-antigen biosynthesis gene cluster” as used herein refers to a group of genes that encode enzymes that are involved in the biosynthesis of the common-antigen comprising genes involved in nucleotide sugar biosynthesis, glycosyltransferases and common-antigen processing genes.

The term “colanic acid biosynthesis gene cluster” as used herein refers to a group of genes that encode enzymes that are involved in the biosynthesis of the colanic acid comprising genes involved in nucleotide sugar biosynthesis, glycosyltransferases and colanic acid processing genes (Scott et al., 2019, Biochem. 58:13, 1818-1830; Stevenson et al., 1996, J. Bacteriol. 178:6, 4885-4893).

The term “purified” refers to material that is substantially or essentially free from components that interfere with the activity of the biological molecule. For cells, saccharides, nucleic acids, and polypeptides, the term “purified” refers to material that is substantially or essentially free from components that normally accompany the material as found in its native state. Typically, purified saccharides, oligosaccharides, proteins or nucleic acids of the disclosure are at least about 50%, 55%, 60%, 65%, 70%, 75%, 80% or 85% pure, usually at least about 90%, 91%, 92%, 93%, 94%, 95%, 96%, 97%, 98%, or 99% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For oligosaccharides, e.g., 3-sialyllactose, purity can be determined using methods such as but not limited to thin layer chromatography, gas chromatography, NMR, HPLC, capillary electrophoresis or mass spectroscopy.

The terms “identical” or percent “identity” or % “identity” in the context of two or more nucleic acid or polypeptide sequences, refer to two or more sequences or subsequences that are the same or have a specified percentage of amino acid residues or nucleotides that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Percent identity may be calculated globally over the full-length sequence of the reference sequence, resulting in a global percent identity score. Alternatively, percent identity may be calculated over a partial sequence of the reference sequence, resulting in a local percent identity score. Using the full-length of the reference sequence in a local sequence alignment results in a global percent identity score between the test and the reference sequence. Percent identity can be determined using different algorithms like, for example, BLAST and PSI-BLAST (Altschul et al., 1990, J. Mol. Biol. 215:3, 403-410; Altschul et al., 1997, Nucleic Acids Res. 25:17, 3389-402), the Clustal Omega method (Sievers et al., 2011, Mol. Syst. Biol. 7:539), the MatGAT method (Campanella et al., 2003, BMC Bioinformatics, 4:29) or EMBOSS Needle (https://galaxy-iuc.github.io/emboss-5.0-docs/needle.html).

The BLAST (Basic Local Alignment Search Tool) method of alignment is an algorithm provided by the National Center for Biotechnology Information (NCBI) to compare sequences using default parameters. The program compares nucleotide or protein sequences to sequence databases and calculates the statistical significance. PSI-BLAST (Position-Specific Iterative Basic Local Alignment Search Tool) derives a position-specific scoring matrix (PSSM) or profile from the multiple sequence alignment of sequences detected above a given score threshold using protein-protein BLAST (BLASTp). The BLAST method can be used for pairwise or multiple sequence alignments. Pairwise Sequence Alignment is used to identify regions of similarity that may indicate functional, structural and/or evolutionary relationships between two biological sequences (protein or nucleic acid). The web interface for BLAST is available at: https://blast.ncbi.nlm.nih.gov/Blast.cgi.

Clustal Omega (Clustal W) is a multiple sequence alignment program that uses seeded guide trees and HMM profile-profile techniques to generate alignments between three or more sequences. It produces biologically meaningful multiple sequence alignments of divergent sequences. The web interface for Clustal W is available at https://www.ebi.ac.uk/Tools/msa/clustalo/. Default parameters for multiple sequence alignments and calculation of percent identity of protein sequences using the Clustal W method are: enabling de-alignment of input sequences: FALSE; enabling mbed-like clustering guide-tree: TRUE; enabling mbed-like clustering iteration: TRUE; Number of (combined guide-tree/HMM) iterations: default(0); Max Guide Tree Iterations: default [−1]; Max H1MM Iterations: default [−1]; order: aligned.

MatGAT (Matrix Global Alignment Tool) is a computer application that generates similarity/identity matrices for DNA or protein sequences without needing pre-alignment of the data. The program performs a series of pairwise alignments using the Myers and Miller global alignment algorithm, calculates similarity and identity, and then places the results in a distance matrix. The user may specify which type of alignment matrix (e.g., BLOSUM50, BLOSUM62, and PAM250) to employ with their protein sequence examination.

EMBOSS Needle (https://galaxy-iuc.github.io/emboss-5.0-docs/needle.html) uses the Needleman-Wunsch global alignment algorithm to find the optimal alignment (including gaps) of two sequences when considering their entire length. The optimal alignment is ensured by dynamic programming methods by exploring all possible alignments and choosing the best. The Needleman-Wunsch algorithm is a member of the class of algorithms that can calculate the best score and alignment in the order of mn steps, (where ‘n’ and ‘m’ are the lengths of the two sequences). The gap open penalty (default 10.0) is the score taken away when a gap is created. The default value assumes you are using the EBLOSUM62 matrix for protein sequences. The gap extension (default 0.5) penalty is added to the standard gap penalty for each base or residue in the gap. This is how long gaps are penalized.

For the purposes of this disclosure, percent identity is determined using MatGAT2.01 (Campanella et al., 2003, BMC Bioinformatics 4:29). The following default parameters for protein are employed: (1) Gap cost Existence: 12 and Extension: 2; (2) The Matrix employed was BLOSUM50.

The term “control sequences” refers to sequences recognized by the host cells transcriptional and translational systems, allowing transcription and translation of a polynucleotide sequence to a polypeptide. Such DNA sequences are thus necessary for the expression of an operably linked coding sequence in a particular host cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. The control sequences can furthermore be controlled with external chemicals, such as, but not limited to, IPTG, arabinose, lactose, allo-lactose, rhamnose or fucose via an inducible promoter or via a genetic circuit that either induces or represses the transcription or translation of the polynucleotide to a polypeptide.

Generally, “operably linked” means that the DNA sequences being linked are contiguous, and, in the case of a secretory leader, contiguous and in reading phase. However, enhancers do not have to be contiguous.

As used herein, the term “cell productivity index (CPI)” refers to the mass of the product produced by the cells divided by the mass of the cells produced in the culture.

The terms “precursor” as used herein refers to substances that are taken up or synthetized by the cell for the specific production of a sialylated oligosaccharide. In this sense a precursor can be an acceptor as defined herein, but can also be another substance, metabolite, which is first modified within the cell as part of the biochemical synthesis route of the sialylated oligosaccharide. Examples of such precursors comprise the acceptors as defined herein, and/or glucose, galactose, fructose, glycerol, sialic acid, fucose, mannose, maltose, sucrose, lactose glucose-1-phosphate, galactose-1-phosphate, UDP-glucose, UDP-galactose, glucose-6-phosphate, fructose-6-phosphate, fructose-1,6-bisphosphate, glycerol-3-phosphate, dihydroxyacetone, glyceraldehyde-3-phosphate, dihydroxyacetone-phosphate, glucosamine-6-phosphate, glucosamine, N-acetyl-glucosamine-6-phosphate, N-acetyl-glucosamine, N-acetyl-mannosamine, N-acetylmannosamine-6-phosphate, UDP-N-acetylglucosamine, N-acetylglucosamine-1-phosphate, N-acetylneuraminic acid (sialic acid), N-acetyl-Neuraminic acid-9 phosphate, CMP-sialic acid, mannose-6-phosphate, mannose-1-phosphate, GDP-mannose, GDP-4-dehydro-6-deoxy-α-D-mannose, and/or GDP-fucose.

The term “acceptor” as used herein refers to oligosaccharides, which can be modified by a sialyltransferase, fucosyltransferase, galactosyltransferase, N-acetylglucosamine transferase, N-acetylgalactosamine transferase. Examples of such acceptors are lactose, lacto-N-biose (LNB), lacto-N-triose, lacto-N-tetraose (LNT), lacto-N-neotetraose (LNnT), N-acetyl-lactosamine (LacNAc), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, galactosyllactose, a lactose extended with 1, 2, 3, 4, 5, or a multiple of N-acetyllactosamine units and/or 1, 2, 3, 4, 5, or a multiple of, Lacto-N-biose units, and oligosaccharide containing 1 or multiple N-acetyllactosamine units and/or 1 or multiple lacto-N-biose units or an intermediate into sialylated oligosaccharide, fucosylated and sialylated versions thereof.

An amino acid sequence or polypeptide sequence or protein sequence, used herein interchangeably, of the polypeptide used herein can be a sequence as indicated with the SEQ ID NO of the attached sequence listing. The amino acid sequence of the polypeptide can also be an amino acid sequence that has 80% or more sequence identity, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9 sequence identity to the full length amino acid sequence of any one of the respective SEQ ID NO.

The term “foaming” as used herein refers to the generation of foam during fermentation processes caused by the existence of foam-active substances in the fermentation broth, escaping gas/air and turbulences within the fermenter. Sugars, starches and proteins, as part of the growth medium the cells are growing in, act as foam promoting substances and they may be assisted by other substances or ingredients that partly consist of trace elements for the microorganisms. Also, amino acids and proteins, which are generated by the microorganisms during the fermentation, can cause considerable foam activity. Foaming can be a serious problem in fermentation, particularly in large scale, highly loaded fermentations, causing overflow and dangerous or inefficient use of the reactor.

The term “airlift” as used herein refers to the gas holdup within the liquid of a chemical or biological fluid, for instance, a biocatalytical mixture or fermentation broth, wherein the gas holdup increases the volume of the liquid by an upward displacement in the reactor, tank or bioreactor.

The term “vessel filling” as used herein refers to the level a bioreactor or reactor or tank is filled in a process relative to the maximum volume a bioreactor, reactor or tank can hold, expressed in percentage. A vessel filling percentage is, for instance, non-limiting higher or equal to 50%; 55%; 60%, 65%, 66%, 67%, 68%, 69%; 70%; 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. The vessel filling is dependent on parameters comprising but not limited to vessel geometry, the volume of the inoculum, volume of the biomass generated upon cultivation of the host, volume of the feeds added during cultivation such as, for example, carbon source feed, precursor feed, acceptor feed, salts feed, acid feed, base feed, antifoam addition.

The term ‘micro-organism’ or ‘cell’ as used herein refers to a microorganism chosen from the list consisting of a bacterium, a yeast or a fungus. The latter bacterium preferably belongs to the phylum of the Proteobacteria or the phylum of the Firmicutes or the phylum of the Cyanobacteria or the phylum Deinococcus-Thermus. The latter bacterium belonging to the phylum Proteobacteria belongs preferably to the family Enterobacteriaceae, preferably to the species Escherichia coli.

Examples of Escherichia strains that can be used include, but are not limited to, Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. The disclosure specifically relates to a mutated and/or transformed Escherichia coli strain as indicated above wherein the E. coli strain is a K12 strain. More specifically, the disclosure relates to a mutated and/or transformed Escherichia coli strain as indicated herein wherein the K12 strain is E. coli substr. MG1655.

Alternatively, the E. coli is selected from the group consisting of K-12 strain, W3110, MG1655, B/r, BL21, O157:h7, 042, 101-1,1180, 1357, 1412, 1520, 1827-70, 2362-75, 3431, 53638, 83972, 929-78, 98NK2, ABU 83972, B, B088, B171, B185, B354, B646, B7A, C, c7122, CFT073, DH1, DH5a, E110019, E128010, E74/68, E851/71, EAEC 042, EPECall, EPECa12, EPECa14, ETEC, H10407, F11, F18+, FVEC1302, FVEC1412, GEMS_EPEC1, HB101, HT 115, KO 11, LF82, LT-41, LT-62, LT-68, MS107-1, MS 119-7, MS124-1, MS 145-7, MS 79-2, MS 85-1, NCTC 86, Nissle 1917, NT:H19, NT:H40, NU14, 0103:H2, 0103:HNM, O103:K+, O104:H12, 0108:H25, 0109:H9, O111H−, O111:H19, 011:H2, 0111:H21, O111:NM, O115:H−, O115:HMN, O115:K+, O119:H6, 0119:UT, O124:H40, 0127a:H6, O127:H6, 0128:H2, 0131:H25, 0136:H−, O139:H28 (strain E24377A/ETEC), O13:H11, O142:H6, O145:H−, O153:H21, O153:H7, O154:H9, 0157:12, O157:H−, O157:H12, O157:H43, O157:H45, O157:H7 EDL933, 0157:NM, O15:NM, O177:H11, 017:K52:H18 (strain UMNO26/ExPEC), O180:H−, OL:K1/APEC, 026, 026:H−, 026:H11, 026:H11:K60, 026:NM, O41:H−, 045:K1 (strain S88/ExPEC), 051:H−, O55:H51, 055:H6, 055:H7, 05:H−, 06, 063:H6, 063:HNM, 06:K15:H31 (strain 536/UPEC), 07:K1 (strain IAI39/ExPEC), 08 (strain IAIl), 081 (strain EDla), 084:H−, 086a:H34, 086a:H40, 090:H8, 091:H21, 09:H4 (strain HS), 09:H51, ONT:H−, ONT:H25, OP50, Orough:H12, Orough:H19, Orough:H34, Orough:H37, Orough:H9, OUT:H12, OUT:H45, OUT:H6, OUT:H7, OUT:HNM, OUT:NM, RN587/1, RS218, 55989/EAEC, B/BL21, B/BL21-DE3, SE11, SMS-3-5/SECEC, UTI89/UPEC, TA004, TA155, TX1999, and Vir68.

The latter bacterium belonging to the phylum Firmicutes belongs preferably to the Bacilli, preferably from the species Bacillus. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces, Pichia, Komagataella, Hansenula, Kluyveromyces, Yarrowia, Eremothecium, Zygosaccharomyces or Debaromyces. The latter fungus belongs preferably to the genus Rhizopus, Dictyostelium or Aspergillus.

### DETAILED DESCRIPTION

In a first embodiment, the disclosure provides a genetically modified micro-organism or cell thereof modified to produce at least one glycosylated product wherein the micro-organism has a reduced cell wall biosynthesis.

The glycosylated product is a product as defined herein. In a preferred embodiment, the glycosylated product is a saccharide, a glycosylated aglycon, a glycolipid or a glycoprotein. Such glycosylated product can be an oligosaccharide with a degree of polymerization higher than 2. In an exemplary embodiment the glycosylated product is an oligosaccharide with a degree of polymerization higher than 3.

Alternatively, such glycosylated product can be any oligosaccharide described herein.

In a preferred embodiment, the cell wall biosynthesis is reduced by a deletion, reduced or abolished expression of at least one enzyme within the cell wall biosynthesis pathway.

In another preferred embodiment, the cell wall biosynthesis is reduced by deletion, reduced or abolished expression of at least one glycosyltransferase within the cell wall biosynthesis pathway.

In another preferred embodiment of the disclosure, the reduced cell wall biosynthesis in the genetically modified micro-organism is combined with the introduction of one or more pathways for the synthesis of one or more nucleotide-activated sugars. Preferably, the nucleotide-activated sugar is chosen from the list comprising UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-N-glycolylneuraminic acid (CMP-Neu5Gc), GDP-fucose (GDP-Fuc), GDP-rhamnose and UDP-xylose.

In a further preferred embodiment of the disclosure, the micro-organism with a reduced cell wall biosynthesis is modified to express one or more glycosyltransferases that is/are involved in the production of a glycosylated product of disclosure. Preferably, the glycosyltransferase is selected from the list comprising but not limited to: fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases and fucosaminyltransferases.

In another preferred embodiment of the disclosure, the reduced cell wall biosynthesis in the genetically modified micro-organism is combined with the introduction of one or more pathways chosen from but not limited to a fucosylation, sialylation, galactosylation, N-acetylglucosaminylation, N-acetylgalactosylation, mannosylation, N-acetylmannosinylation pathway as described herein.

The micro-organism or cell of the disclosure can be any bacterium or yeast, preferably as described herein. The bacterium can be a Gram-positive bacterium or Gram-negative bacterium. Examples of Gram-negative bacteria useful in the disclosure include, but are not limited to of Escherichia spp., Shigella spp., Salmonella spp., Campylobacter spp., Neisseria spp., Haemophilus spp., Aeromonas spp., Francisella spp., Yersinia spp., Klebsiella spp., Bordetella spp., Legionella spp., Citrobacter spp., Chlamydia spp., Brucella spp., Pseudomonas spp., Helicobacter spp., Moraxella spp., Stenotrophomonas spp., Bdellovibrio spp., Acinetobacter spp., Enterobacter spp. and Vibrio spp. Examples of Gram-positive bacteria comprise, but are not limited to, Bacillus, Lactobacillus, Lactococcus. Examples of yeast comprise, but are not limited to, Pichia, Hansenula, Komagataella, Saccharomyces.

In another preferred embodiment, the cell wall biosynthesis pathway is at least one pathway chosen from cell wall carbohydrate antigen biosynthesis, preferably O-antigen and/or common-antigen biosynthesis when the micro-organism is a Gram-negative bacterium; capsular polysaccharide biosynthesis; cell wall protein mannosylation biosynthesis, beta-1,3-glucan biosynthesis, beta-1,6-glucan biosynthesis and/or chitin biosynthesis when the micro-organism is a yeast; mycolic acid and/or arabinogalactan biosynthesis when the micro-organism is a Corynebacterium, Nocardia or Mycobacterium; or teichoic acid biosynthesis when the micro-organism is a Gram-positive bacterium, preferably Bacillus.

According to a further preferred embodiment of the disclosure, the micro-organism is a bacterium with a further cell wall biosynthesis pathway that is reduced by a deletion, reduced or abolished expression of at least one enzyme within the further cell wall biosynthesis pathway chosen from colanic acid biosynthesis, exopolysaccharide biosynthesis and/or lipopolysaccharide biosynthesis.

The micro-organism or cell according to the disclosure can be a Gram-negative bacterium modified in cell wall carbohydrate antigen biosynthesis, preferably the O-antigen biosynthesis and/or the common antigen biosynthesis.

In a preferred embodiment, the Gram-negative bacterium has a modified 0-antigen biosynthesis, which is provided by a deletion, reduced or abolished expression of any one or more of the genes present in the O-antigen biosynthesis gene cluster comprising rhamnosyltransferase, putative annotated glycosyltransferase, putative lipopolysaccharide biosynthesis O-acetyl transferase, β-1,6-galactofuranosyltransferase, putative O-antigen polymerase, UDP-galactopyranose mutase, polyisoprenol-linked O-antigen repeat unit flippase, dTDP-4-dehydrorhamnose 3,5-epimerase, dTDP-glucose pyrophosphorylase, dTDP-4-dehydrorhamnose reductase, dTDP-glucose 4,6-dehydratase 1, UTP:glucose-1-phosphate uridylyltransferase. Alternatively, the modification in the O-antigen biosynthesis is provided by a deletion, reduced or abolished expression of any one or more of i) WbbL, WbbK, WbbJ, WbbI, WbH, glf, rfbX, rfbC, rfbA, rfbD, rfbB, wcaN, preferably as given by SEQ ID NOs:27 to 38, respectively, or ii) a polypeptide sequence having 80% or more sequence identity to the full-length sequence of any one of the SEQ ID NOs:27 to 38 and having rhamnosyltransferase activity, annotated glycosyltransferase activity, lipopolysaccharide biosynthesis O-acetyl transferase activity, β-1,6-galactofuranosyltransferase activity, O-antigen polymerase activity, UDP-galactopyranose mutase activity, polyisoprenol-linked O-antigen repeat unit flippase activity, dTDP-4-dehydrorhamnose 3,5-epimerase activity, dTDP-glucose pyrophosphorylase activity, dTDP-4-dehydrorhamnose reductase activity, dTDP-glucose 4,6-dehydratase 1 activity or UTP:glucose-1-phosphate uridylyltransferase activity, respectively.

In another preferred embodiment, the Gram-negative bacterium has a modified O-antigen biosynthesis pathway combined with the introduction of one or more pathways chosen from but not limited to a fucosylation, sialylation, galactosylation, N-acetylglucosaminylation, N-acetylgalactosylation, mannosylation, N-acetylmannosinylation pathway as described herein.

In still another preferred embodiment, the Gram-negative bacterium has a modified common-antigen biosynthesis, which is provided by a deletion, reduced or abolished expression of in any one or more of the genes present in the common-antigen biosynthesis gene cluster comprising UDP-N-acetylglucosamine-undecaprenyl-phosphate N-acetylglucosaminephosphotransferase, enterobacterial common antigen polysaccharide co-polymerase, UDP-N-acetylglucosamine 2-epimerase, UDP-N-acetyl-D-mannosamine dehydrogenase, dTDP-glucose 4,6-dehydratase 2, dTDP-glucose pyrophosphorylase, dTDP-4-amino-4,6-dideoxy-D-galactose acyltransferase, dTDP-4-dehydro-6-deoxy-D-glucose transaminase, lipid III flippase, TDP-N-acetylfucosamine:lipid II N-acetylfucosaminyltransferase, putative enterobacterial common antigen polymerase, UDP-N-acetyl-D-mannosaminuronic acid transferase. Alternatively, the modification in the common-antigen biosynthesis is provided by a deletion, reduced or abolished expression of any one or more of i) rfe, wzzE, wecB, wecC, rffG, rfH, rffC, wecE, wzxE, wecF, wzyE, rffM, preferably as given by SEQ ID NOs:15 to 26, respectively, or ii) a polypeptide sequence having 80% or more sequence identity to the full-length sequence of any one of the SEQ ID NOs:15 to 26 and having UDP-N-acetylglucosamine-undecaprenyl-phosphate N-acetylglucosaminephosphotransferase activity, enterobacterial common antigen polysaccharide co-polymerase activity, UDP-N-acetylglucosamine 2-epimerase activity, UDP-N-acetyl-D-mannosamine dehydrogenase activity, dTDP-glucose 4,6-dehydratase 2 activity, dTDP-glucose pyrophosphorylase activity, dTDP-4-amino-4,6-dideoxy-D-galactose acyltransferase activity, dTDP-4-dehydro-6-deoxy-D-glucose transaminase activity, lipid III flippase activity, TDP-N-acetylfucosamine:lipid II N-acetylfucosaminyltransferase activity, enterobacterial common antigen polymerase activity or UDP-N-acetyl-D-mannosaminuronic acid transferase activity, respectively.

In another preferred embodiment, the Gram-negative bacterium has a modified common-antigen biosynthesis pathway combined with the introduction of one or more pathways chosen from but not limited to a fucosylation, sialylation, galactosylation, N-acetylglucosaminylation, N-acetylgalactosylation, mannosylation, N-acetylmannosinylation pathway as described herein.

In a further preferred embodiment, the micro-organism is a bacterium having a further reduced cell wall biosynthesis by a reduced colanic acid biosynthesis wherein the reduction in the colanic acid biosynthesis is provided by a deletion, reduced or abolished expression of any one or more of the genes present in the colanic acid biosynthesis gene cluster. In an exemplary embodiment thereof, the modification in the colanic acid biosynthesis is provided by a deletion, reduced or abolished expression of any one or more of the genes present in the colanic acid biosynthesis gene cluster comprising putative colanic acid biosynthesis protein, putative colanic biosynthesis glycosyl transferase, putative colanic acid biosynthesis pyruvyl transferase, M-antigen undecaprenyl diphosphate flippase, UDP-glucose:undecaprenyl-phosphate glucose-1-phosphate transferase, phosphomannomutase, mannose-1-phosphate guanylyltransferase, colanic acid biosynthesis fucosyltransferase, GDP-mannose mannosyl hydrolase, GDP-L-fucose synthase, GDP-mannose 4,6-dehydratase, colanic acid biosynthesis acetyltransferase, colanic acid biosynthesis fucosyltransferase, putative colanic acid polymerase, colanic acid biosynthesis galactosyltransferase, colanic acid biosynthesis acetyltransferase, colanic acid biosynthesis glucuronosyltransferase, protein-tyrosine kinase, protein-tyrosine phosphatase, outer membrane polysaccharide export protein. Alternatively, the modification in the colanic acid biosynthesis is provided by a deletion, reduced or abolished expression of any one or more of i) WcaM, WcaL, WcaK, WzxC, wcaJ, cpsG, cpsB, WcaI, gmm, fcl, gmd, WcaF, WcaE, WcaD, WcaC, WcaB, WcaA, Wzc, wzb, Wza, preferably as given by SEQ ID NOs:39 to 58, respectively, or ii) a polypeptide sequence having 80% or more sequence identity to the full-length sequence of any one of the SEQ ID NOs:39 to 58 and having colanic acid biosynthesis protein activity, colanic biosynthesis glycosyl transferase activity, colanic acid biosynthesis pyruvyl transferase activity, M-antigen undecaprenyl diphosphate flippase activity, UDP-glucose:undecaprenyl-phosphate glucose-1-phosphate transferase activity, phosphomannomutase activity, mannose-1-phosphate guanylyltransferase activity, colanic acid biosynthesis fucosyltransferase activity, GDP-mannose mannosyl hydrolase activity, GDP-L-fucose synthase activity, GDP-mannose 4,6-dehydratase activity, colanic acid biosynthesis acetyltransferase activity, colanic acid biosynthesis fucosyltransferase activity, colanic acid polymerase activity, colanic acid biosynthesis galactosyltransferase activity, colanic acid biosynthesis acetyltransferase activity, colanic acid biosynthesis glucuronosyltransferase activity, protein-tyrosine kinase activity, protein-tyrosine phosphatase activity or outer membrane polysaccharide export protein activity, respectively.

In another preferred embodiment, the bacterium having a further reduced cell wall biosynthesis by a reduced colanic acid biosynthesis is modified by the introduction of one or more pathways chosen from but not limited to a fucosylation, sialylation, galactosylation, N-acetylglucosaminylation, N-acetylgalactosylation, mannosylation, N-acetylmannosinylation pathway as described herein.

In another exemplary embodiment of the disclosure, the micro-organism is a yeast modified in the cell wall protein mannosylation biosynthesis, Beta1,3 glucan biosynthesis, beta 1,6 glucan biosynthesis and/or chitin biosynthesis.

In a further exemplary embodiment, the micro-organism is a yeast modified in the cell wall protein mannosylation biosynthesis, Beta1,3 glucan biosynthesis, beta 1,6 glucan biosynthesis and/or chitin biosynthesis and further modified by the introduction of one or more pathways chosen from but not limited to a fucosylation, sialylation, galactosylation, N-acetylglucosaminylation, N-acetylgalactosylation, mannosylation, N-acetylmannosinylation pathway as described herein.

In a preferred exemplary embodiment of the disclosure, the micro-organism is a yeast having a reduced cell wall biosynthesis by a reduced cell wall protein mannosylation biosynthesis. Preferably, the reduction in the cell wall protein mannosylation biosynthesis is provided by a deletion, reduced or abolished expression of any one or more of Protein-O-mannosyltransferase, preferably one or more of PMT1, PMT2, PMT3, PMT4, PMT5, PMT6, PMT7, more preferably one or more of PMT1, PMT2, PMT4.

In still another preferred embodiment of the disclosure, the micro-organism is a Corynebacterium, Nocardia or Mycobacterium modified in the expression of any one or more of mycolic acid biosynthesis, and/or arabinogalactan biosynthesis. Preferably, modified in the expression of any one or more of accD2, accD3, aftA, aftB or emb. In a more preferred embodiment of the disclosure, the micro-organism is a Corynebacterium, Nocardia or Mycobacterium having a reduced cell wall biosynthesis by a reduced mycolic acid and/or arabinogalactan biosynthesis. Preferably, the reduced mycolic acid and/or arabinogalactan biosynthesis is provided by a reduced expression of any one or more of mycolic acid and/or arabinogalactan biosynthesis genes, more preferably by reduced expression of any one or more of accD2, accD3, aftA, aftB or emb.

In a further preferred embodiment of the disclosure, the micro-organism is a Corynebacterium, Nocardia or Mycobacterium modified in the expression of any one or more of mycolic acid biosynthesis, and/or arabinogalactan biosynthesis and further modified by the introduction of one or more pathways chosen from but not limited to a fucosylation, sialylation, galactosylation, N-acetylglucosaminylation, N-acetylgalactosylation, mannosylation, N-acetylmannosinylation pathway as described herein.

In another preferred embodiment of the disclosure, the micro-organism is a Gram-positive bacterium modified in the expression of teichoic acid biosynthesis. Preferably, modified in the expression of any one or more of tagO, tagA, tagB, tagD, tagF, tagG or tagH.

In a more preferred embodiment, the micro-organism is a Gram-positive bacterium having a reduced cell wall biosynthesis by a reduced teichoic acid biosynthesis. Preferably, the reduced teichoic acid biosynthesis is provided by a reduced expression of any one or more of teichoic acid biosynthesis genes, more preferably by reduced expression of any one or more of tagO, tagA, tagB, tagD, tagF, tagG or tagH.

In a further preferred embodiment of the disclosure, the micro-organism is a Gram-positive bacterium having a reduced cell wall biosynthesis by a reduced teichoic acid biosynthesis and further modified by the introduction of one or more pathways chosen from but not limited to a fucosylation, sialylation, galactosylation, N-acetylglucosaminylation, N-acetylgalactosylation, mannosylation, N-acetylmannosinylation pathway as described herein.

According to the disclosure, the micro-organism can be an isolated micro-organism according to any of the micro-organisms described herein.

Ina second embodiment, the disclosure provides a method to reduce the viscosity, foaming, and/or airlift of a fermentation process with a micro-organism characterized in that the cell wall biosynthesis of the micro-organism is modified, preferably reduced cell wall biosynthesis. Preferably, the cell wall biosynthesis of the micro-organism is reduced by deletion, reduced or abolished expression of at least one enzyme within the cell wall biosynthesis pathway. More preferably, the micro-organism is a bacterium or yeast and the cell wall biosynthesis pathway is at least one pathway chosen from: cell wall carbohydrate antigen biosynthesis, preferably O-antigen and/or common-antigen biosynthesis when the micro-organism is a Gram-negative bacterium; capsular polysaccharide biosynthesis; cell wall protein mannosylation biosynthesis, beta-1,3-glucan biosynthesis, beta-1,6-glucan biosynthesis and/or chitin biosynthesis when the micro-organism is a yeast; mycolic acid and/or arabinogalactan biosynthesis when the micro-organism is a Corynebacterium, Nocardia or Mycobacterium or teichoic acid biosynthesis when the micro-organism is a Gram-positive bacterium, preferably Bacillus. Preferably, the micro-organism is further modified to produce at least one glycosylated product as described herein.

In a third embodiment, the disclosure provides a method for the production of a glycosylated product by a genetically modified cell, comprising the steps of:
providing a cell genetically modified for the production of glycosylated product, the cell comprising at least one nucleic acid sequence coding for an enzyme for glycosylated product synthesis,
the cell further genetically modified for reduced cell wall biosynthesis, by deletion, reduced or abolished expression of at least one enzyme within the cell wall biosynthesis pathway, wherein the cell wall biosynthesis pathway is at least one pathway chosen from cell wall carbohydrate antigen biosynthesis, capsular polysaccharide biosynthesis, cell wall protein mannosylation biosynthesis, beta-1,3-glucan biosynthesis, beta-1,6-glucan biosynthesis, chitin biosynthesis, mycolic acid biosynthesis, arabinogalactan biosynthesis and teichoic acid biosynthesis, preferably wherein the cell wall carbohydrate antigen biosynthesis is O-antigen and/or common-antigen biosynthesis,
culturing the cell in a medium under conditions permissive for the production of glycosylated product,
optionally separating glycosylated product from the culture.

The genetically modified cell is any micro-organism as described herein. Preferably bacterium or yeast. More preferably, the genetically modified cell is bacterium, preferably Enterobacteriaceae, more preferably Escherichia as described herein. In another more preferred embodiment, the genetically modified cell is yeast, preferably Pichia, Hansenula, Komagataella or Saccharomyces.

Another embodiment of the disclosure provides a method for the production of glycosylated product by a genetically modified Gram-negative bacterial cell. A Gram-negative bacterial cell genetically modified for the production of glycosylated product is provided wherein the cell comprises at least one nucleic acid sequence coding for an enzyme for glycosylated product synthesis. The enzyme for glycosylated product synthesis comprises enzymes involved in nucleotide-activated sugar synthesis and glycosyltransferases as described herein. The cell is further genetically modified for reduced cell wall biosynthesis by deletion, reduced or abolished expression of at least one enzyme within the cell wall biosynthesis pathway, the cell wall biosynthesis being cell wall carbohydrate antigen biosynthesis, preferably O-antigen and/or common-antigen biosynthesis. This cell is cultured in a medium under conditions permissive for the production of glycosylated product. Optionally, the glycosylated product can be separated from the culture.

In a further preferred embodiment, the disclosure provides a method for the production of glycosylated product by a genetically modified Gram-negative bacterial cell that has a further cell wall biosynthesis pathway that is reduced by a deletion, reduced or abolished expression of at least one enzyme within the further cell wall biosynthesis pathway. Herein, the further cell wall biosynthesis pathway is colanic acid biosynthesis, exopolysaccharide biosynthesis and/or lipopolysaccharide biosynthesis.

Another exemplary embodiment of the disclosure provides a method for the production of glycosylated product by a genetically modified yeast cell. Here, a yeast cell genetically modified for the production of glycosylated product is provided wherein the cell comprises at least one nucleic acid sequence coding for an enzyme for glycosylated product synthesis. The enzyme for glycosylated product synthesis comprises enzymes involved in nucleotide-activated sugar synthesis and glycosyltransferases as described herein. The cell is further genetically modified for reduced cell wall biosynthesis by deletion, reduced or abolished expression of at least one enzyme within the cell wall biosynthesis pathway, the cell wall biosynthesis being i) cell wall protein mannosylation biosynthesis, ii) beta-1,3-glucan biosynthesis, iii) beta-1,6-glucan biosynthesis, and/or iv) chitin biosynthesis. The cell is cultured in a medium under conditions permissive for the production of glycosylated product. Optionally, the glycosylated product is separated from the culture.

Another exemplary embodiment of the disclosure provides a method for the production of glycosylated product by a genetically modified Corynebacterium, Nocardia or Mycobacterium cell. Here, a Corynebacterium, Nocardia or Mycobacterium cell genetically modified for the production of glycosylated product is provided wherein the cell comprises at least one nucleic acid sequence coding for an enzyme for glycosylated product synthesis. The enzyme for glycosylated product synthesis comprises enzymes involved in nucleotide-activated sugar synthesis and glycosyltransferases as described herein. The cell is further genetically modified for reduced cell wall biosynthesis by deletion, reduced or abolished expression of at least one enzyme within the cell wall biosynthesis pathway, the cell wall biosynthesis being i) mycolic acid biosynthesis, and/or ii) arabinogalactan biosynthesis. The cell is cultured in a medium under conditions permissive for the production of glycosylated product. Optionally, the glycosylated product is separated from the culture.

Another exemplary embodiment of the disclosure provides a method for the production of glycosylated product by a genetically modified Bacillus cell. A Bacillus cell genetically modified for the production of glycosylated product is provided wherein the cell comprising at least one nucleic acid sequence coding for an enzyme for glycosylated product synthesis. The enzyme for glycosylated product synthesis comprises enzymes involved in nucleotide-activated sugar synthesis and glycosyltransferases as described herein. The cell is further genetically modified for reduced cell wall biosynthesis by deletion, reduced or abolished expression of at least one enzyme within the cell wall biosynthesis pathway, the cell wall biosynthesis being teichoic acid biosynthesis. The cell is cultured in a medium under conditions permissive for the production of glycosylated product. Optionally, the glycosylated product is separated from the culture.

In a preferred embodiment of the methods described herein, the cell wall biosynthesis is reduced by deletion, reduced or abolished expression of at least one glycosyltransferase within the cell wall biosynthesis pathway.

As described herein, a method for the production of glycosylated product by any cell from a micro-organism as described herein can be used for the method. Such cell is then cultured in a medium under conditions permissive for the production of the glycosylated product. Optionally, the glycosylated product is separated from the culture.

In the methods of the disclosure the glycosylated product, e.g., an oligosaccharide, can be isolated from the culture medium by means of unit operation selected from the group comprising centrifugation, filtration, microfiltration, ultrafiltration, nanofiltration, ion exchange, electrodialysis, chromatography, simulated moving bed chromatography, simulated moving bed ion exchange, evaporation, precipitation, crystallisation, spray drying and any combination thereof.

In an exemplary preferred embodiment of the methods of the invention the produced oligosaccharide or mix of oligosaccharides is separated from the culture.

As used herein, the term “separating” means harvesting, collecting or retrieving the glycosylated product from the host cell and/or the medium of its growth as explained herein.

Glycosylated product, e.g., oligosaccharide, can be separated in a conventional manner from the culture or aqueous culture medium, in which the mixture was made. In case an glycosylated product is still present in the cells producing the glycosylated product, conventional manners to free or to extract the glycosylated product out of the cells can be used, such as cell destruction using high pH, heat shock, sonication, French press, homogenisation, enzymatic hydrolysis, chemical hydrolysis, solvent hydrolysis, detergent, hydrolysis, etc. The culture medium, reaction mixture and/or cell extract, together and separately called glycosylated product containing mixture or culture, can then be further used for separating the glycosylated product.

Typically oligosaccharides are purified by first removing macro components, i.e., first the cells and cell debris, then the smaller components, i.e., proteins, endotoxins and other components between 1000 Da (Dalton) and 1000 kDa and then the oligosaccharide is desalted by means of retaining the oligosaccharide with a nanofiltration membrane or with electrodialysis in a first step and ion exchange in a second step, which consists of a cation exchange resin and anion exchange resin, wherein most preferably the cation exchange chromatography is performed before the anion exchange chromatography. These steps do not separate sugars with a small difference in degree of polymerization from each other. The separation is done, for instance, by chromatographical separation.

Separation preferably involves clarifying the glycosylated product containing mixtures to remove suspended particulates and contaminants, particularly cells, cell components, insoluble metabolites and debris produced by culturing the genetically modified cell and/or performing the enzymatic reaction. In this step, the glycosylated product containing mixture can be clarified in a conventional manner. Preferably, the glycosylated product containing mixture is clarified by centrifugation, flocculation, decantation and/or filtration. A second step of separating the glycosylated product from the glycosylated product containing mixture preferably involves removing substantially all the proteins, as well as peptides, amino acids, RNA and DNA and any endotoxins and glycolipids that could interfere with the subsequent separation step, from the glycosylated product containing mixture, preferably after it has been clarified. In this step, proteins and related impurities can be removed from the glycosylated product containing mixture in a conventional manner. Preferably, proteins, salts, by-products, color and other related impurities are removed from the glycosylated product containing mixture by ultrafiltration, nanofiltration, reverse osmosis, microfiltration, activated charcoal or carbon treatment, tangential flow high-performance filtration, tangential flow ultrafiltration, affinity chromatography, ion exchange chromatography (such as but not limited to cation exchange, anion exchange, mixed bed ion exchange), hydrophobic interaction chromatography and/or gel filtration (i.e., size exclusion chromatography), particularly by chromatography, more particularly by ion exchange chromatography or hydrophobic interaction chromatography or ligand exchange chromatography. With the exception of size exclusion chromatography, proteins and related impurities are retained by a chromatography medium or a selected membrane, while glycosylated product remains in the glycosylated product containing mixture.

Contaminating compounds with a molecular weight above 1000 Da are removed by means of ultrafiltration membranes with a cut-off above 1000 Da to approximately 1000 kDa. The membrane retains the contaminant and the glycosylated product goes to the filtrate. Typical ultrafiltration principles are well known in the art and are based on Tubular modules, Hollow fiber, spiral-wound or plates. These are used in cross flow conditions or as a dead-end filtration. The membrane composition is well known and available from several vendors, and are composed of PES (Polyethylene sulfone), polyvinylpyrrolidone, PAN (Polyacrylonitrile), PA (Poly-amide), Polyvinylidene difluoride (PVDF), NC (Nitrocellulose), ceramic materials or combinations thereof.

Components smaller than the glycosylated product, for instance, monosaccharides, salts, disaccharides, acids, bases, medium constituents are separated by means of a nano-filtration or/and electrodialysis. Such membranes have molecular weight cut-offs between 100 Da and 1000 Da. For an oligosaccharide such as 3′-sialyllactose or 6′-sialyllactose the optimal cut-off is between 300 Da and 500 Da, minimizing losses in the filtrate. Typical membrane compositions are well known and are, for example, polyamide (PA), TFC, PA-TFC, Polypiperazine-amide, PES, Cellulose Acetate or combinations thereof.

The glycosylated product is further isolated from the culture medium and/or cell with or without further purification steps by evaporation, lyophilization, crystallization, precipitation, and/or drying, spray drying. The further purification steps allow the formulation of glycosylated product in combination with other glycosylated product and/or products, for instance, but not limited to the co-formulation by means of spray drying, drying or lyophilization or concentration by means of evaporation in liquid form.

In an even further aspect, the disclosure also provides for a further purification of the glycosylated product. A further purification of the glycosylated product may be accomplished, for example, by use of (activated) charcoal or carbon, nanofiltration, ultrafiltration or ion exchange to remove any remaining DNA, protein, LPS, endotoxins, or other impurity. Alcohols, such as ethanol, and aqueous alcohol mixtures can also be used. Another purification step is accomplished by crystallization or precipitation of the product. Another purification step is to spray dry or lyophilize oligosaccharide.

The separated and preferably also purified glycosylated product, e.g., a mammalian milk oligosaccharide can be used as a supplement in infant formulas and for treating various diseases in new-born infants.

In a specific embodiment an oligosaccharide is produced by the cell according to any one of embodiments described herein and/or according to the method described in any one of embodiments described herein. The oligosaccharide is added to food formulation, feed formulation, pharmaceutical formulation, cosmetic formulation, or agrochemical formulation.

According to the invention, the glycosylated product produced by the methods disclosed herein can be any glycosylated product described herein. Examples of such products comprise saccharide, a glycosylated aglycon, a glycolipid or a glycoprotein. Preferably, the glycosylated product is an oligosaccharide, preferably a mammalian milk oligosaccharide. In another preferred embodiment, the glycosylated product is an oligosaccharide, preferably an oligosaccharide with a degree of polymerization higher than 3.

According to the methods of the disclosure, the reduced cell wall biosynthesis is obtained by modified expression of any one or more of the glycosyltransferases as described herein and wherein that modified expression is obtained by deletion, reduced expression or abolished expression of any one or more of the glycosyltransferases.

The disclosure provides for use of a micro-organism as disclosed herein, in a method for the production of a glycosylated product as described herein. Preferably such glycosylated product is an oligosaccharide, preferably a mammalian milk oligosaccharide.

In a fourth embodiment, the disclosure provides a method for the production of a glycosylated product by a genetically modified cell in a bioreactor. First a cell genetically modified for the production of glycosylated product is provided, wherein the cell comprising at least one nucleic acid sequence coding for an enzyme for glycosylated product synthesis. The enzyme for glycosylated product synthesis comprises enzymes involved in nucleotide-activated sugar synthesis and glycosyltransferases as described herein. This cell is cultured in a medium under conditions permissive for the production of glycosylated product. The cell is cultured in a vessel of a bioreactor wherein the vessel filling of the bioreactor is equal to or higher than 50%. Preferably, the cell used for culturing is a cell of a micro-organism as described herein.

In the methods described herein the glycosylated product can by any glycosylated product as described herein. Preferably the glycosylated product is an oligosaccharide, preferably a mammalian milk oligosaccharide, more preferably chosen from the group of fucosylated oligosaccharide, neutral oligosaccharide or sialylated oligosaccharide as described herein, most preferably chosen from 2′-fucosyllactose, 3-fucosyllactose, difucosyllactose, Lacto-N-tetraose, Lacto-N-neotetraose, 3′-sialyllactose, 6′-sialyllactose, lacto-N-fucopentaose II, lacto-N-fucopentaose I, lacto-N-fucopentaose III, lacto-N-fucopentaose V, lacto-N-fucopentaose VI, sialyllacto-N-tetraose a (LSTd), sialyllacto-N-tetraose c (LSTc), sialyllacto-N-tetraose b (LSTb), sialyllacto-N-tetraose a (LSTa).

Moreover, the disclosure relates to the following specific embodiments:

1. A genetically modified micro-organism modified to produce at least one glycosylated product characterized in that the micro-organism has a reduced cell wall biosynthesis.

2. The modified micro-organism of embodiment 1, wherein the glycosylated product is a saccharide, a glycosylated aglycon, a glycolipid or a glycoprotein.

3. The modified micro-organism of any one of embodiment 1 or 2, wherein the cell wall biosynthesis is reduced by deletion, reduced expression or abolished expression of at least one glycosyltransferase within the cell wall biosynthesis pathway.

4. The modified micro-organism of any one of embodiment 1 to 3, wherein the micro-organism is a bacterium or yeast.

5. The modified micro-organism of any one of embodiment 1 to 4, wherein the micro-organism is an Escherichia, Bacillus, Lactobacillus, Lactococcus, Corynebacterium; or Pichia, Hansenula, Komagataella, Saccharomyces.

6. The modified micro-organism of any one of embodiment 1 to 5, wherein the micro-organism is a bacterium modified in the outer membrane oligosaccharide biosynthesis, exopolysaccharide biosynthesis and/or capsular polysaccharide biosynthesis.

7. The modified micro-organism of any one of embodiment 1 to 6, wherein the micro-organism is a Gram-negative bacterium modified in the lipopolysaccharide biosynthesis.

8. The modified micro-organism of any one of embodiment 1 to 7, wherein the micro-organism is a Gram-negative bacterium modified in the colanic acid biosynthesis, the O-antigen biosynthesis and/or the common antigen biosynthesis.

9. Micro-organism according to embodiment 8, wherein the modification in the colanic acid biosynthesis is provided by a deletion, reduced or abolished expression of any one or more of putative colanic acid biosynthesis protein, putative colanic biosynthesis glycosyl transferase, putative colanic acid biosynthesis pyruvyl transferase, M-antigen undecaprenyl diphosphate flippase, UDP-glucose:undecaprenyl-phosphate glucose-1-phosphate transferase, phosphomannomutase, mannose-1-phosphate guanylyltransferase, colanic acid biosynthesis fucosyltransferase, GDP-mannose mannosyl hydrolase, GDP-L-fucose synthase, GDP-mannose 4,6-dehydratase, colanic acid biosynthesis acetyltransferase, colanic acid biosynthesis fucosyltransferase, putative colanic acid polymerase, colanic acid biosynthesis galactosyltransferase, colanic acid biosynthesis acetyltransferase, colanic acid biosynthesis glucuronosyltransferase, protein-tyrosine kinase, protein-tyrosine phosphatase, outer membrane polysaccharide export protein.

10. Micro-organism according to embodiment 8, wherein said modification in the colanic acid biosynthesis is provided by a deletion, reduced or abolished expression of any one or more of i) WcaM, WcaL, WcaK, WzxC, wcaJ, cpsG, cpsB, WcaI, gmm, fcl, gmd, WcaF, WcaE, WcaD, WcaC, WcaB, WcaA, Wzc, wzb, Wza, preferably as given by SEQ ID NOs:39 to 58, respectively, or ii) a polypeptide sequence having 80% or more sequence identity to any one of the SEQ ID NOs:39 to 58.

Micro-organism according to embodiment 8, wherein the modification in the O-antigen biosynthesis is provided by a deletion, reduced or abolished expression of in any one or more of rhamnosyltransferase, putative glycosyltransferase, putative lipopolysaccharide biosynthesis O-acetyl transferase, β-1,6-galactofuranosyltransferase, putative O-antigen polymerase, UDP-galactopyranose mutase, polyisoprenol-linked O-antigen repeat unit flippase, dTDP-4-dehydrorhamnose 3,5-epimerase, dTDP-glucose pyrophosphorylase, dTDP-4-dehydrorhamnose reductase, dTDP-glucose 4,6-dehydratase 1, UTP:glucose-1-phosphate uridylyltransferase.

12. Micro-organism according to embodiment 8, wherein the modification in the O-antigen biosynthesis is provided by a deletion, reduced or abolished expression of any one or more of i) WbbL, WbbK, WbbJ, WbbI, WbbH, glf, rfbX, rfbC, rfbA, rfbD, rfbB, wcaN, preferably as given by SEQ ID NOs:27 to 38, respectively, or ii) a polypeptide sequence having 80% or more sequence identity to any one of the SEQ ID NOs:27 to 38.

Micro-organism according to embodiment 8, wherein the modification in the common-antigen biosynthesis is provided by a deletion, reduced or abolished expression of in any one or more of UDP-N-acetylglucosamine-undecaprenyl-phosphate N-acetylglucosaminephosphotransferase, enterobacterial common antigen polysaccharide co-polymerase, UDP-N-acetylglucosamine 2-epimerase, UDP-N-acetyl-D-mannosamine dehydrogenase, dTDP-glucose 4,6-dehydratase 2, dTDP-glucose pyrophosphorylase, dTDP-4-amino-4,6-dideoxy-D-galactose acyltransferase, dTDP-4-dehydro-6-deoxy-D-glucose transaminase, lipid III flippase, TDP-N-acetylfucosamine:lipid II N-acetylfucosaminyltransferase, putative enterobacterial common antigen polymerase, UDP-N-acetyl-D-mannosaminuronic acid transferase.

14. Micro-organism according to embodiment 8, wherein the modification in the common-antigen biosynthesis is provided by a deletion, reduced or abolished expression of any one or more of i) rfe, wzzE, wecB, wecC, rffG, rffH, rffC, wecE, wzxE, wecF, wzyE, rftM, preferably as given by SEQ ID NOs:15 to 26, respectively, or ii) a polypeptide sequence having 80% or more sequence identity to any one of the SEQ ID NOs:15 to 26.

15. The modified micro-organism according to any one of embodiment 1 to 6, wherein the micro-organism is a yeast modified in the cell wall protein mannosylation biosynthesis, beta1,3 glucan biosynthesis; beta 1,6 glucan biosynthesis and/or chitin biosynthesis.

16. Micro-organism according to embodiment 15, wherein the modification in the cell wall protein mannosylation biosynthesis is provided by a deletion, reduced or abolished expression of any one or more of Protein-O-mannosyltransferase encoding genes, preferably one or more of PMT1, PMT2, PMT3, PMT4, PMT5, PMT6, PMT7, more preferably one or more of PMT1, PMT2, PMT4.

17. The modified micro-organism according to any one of embodiment 1 to 6, wherein the micro-organism is a Corynebacterium, Nocardia or Mycobacterium modified in the expression of any one or more of mycolic acid biosynthesis, and/or arabinogalactan biosynthesis, preferably by modified expression of any one or more of accD2, accD3, aftA, aftB or emb.

18. The modified micro-organism according to any one of embodiment 1 to 6, wherein the micro-organism is a Gram-positive bacterium modified in the expression of teichoic acid biosynthesis, preferably modified in the expression of any one or more of tagO, tagA, tagB, tagD, tagF, tagG or tagH.

19. The modified micro-organism according to any one of embodiment 1 to 18, wherein the glycosylated product is an oligosaccharide with a degree of polymerization higher than 3.

20. Isolated micro-organism according to any one of embodiment 1 to 19.

21. A method to reduce the viscosity, foaming, and/or airlift of a fermentation process with a micro-organism characterized in that the cell wall biosynthesis of the micro-organism is modified, preferably reduced cell wall biosynthesis.

22. The method of embodiment 21, wherein the micro-organism is further modified to produce at least one glycosylated product.

23. Method for the production of glycosylated product by a genetically modified cell, comprising the steps of:
providing a cell genetically modified for the production of glycosylated product, the cell comprising at least one nucleic acid sequence coding for an enzyme for glycosylated product synthesis,
the cell further genetically modified for reduced cell wall biosynthesis,
culturing the cell in a medium under conditions permissive for the production of glycosylated product,
optionally separating glycosylated product from the culture.

24. Method according to embodiment 23, wherein the genetically modified cell is a micro-organism, preferably bacterium or yeast.

25. Method according to any one of embodiment 23 or 24, wherein the genetically modified cell is bacterium, preferably Enterobacteriaceae, more preferably Escherichia.

26. Method according to any one of embodiment 23 or 24, wherein the genetically modified cell is yeast, preferably Saccharomyces or Komagataella.

27. Method for the production of glycosylated product by a genetically modified Gram-negative bacterial cell, comprising the steps of:
providing a Gram-negative bacterial cell genetically modified for the production of glycosylated product, the cell comprising at least one nucleic acid sequence coding for an enzyme for glycosylated product synthesis,
the cell further genetically modified for i) modified expression of colanic acid, ii) modified expression of O-antigen, iii) modified expression of common antigen, and/or iv) modified expression of lipopolysaccharide providing reduced cell wall biosynthesis.
culturing the cell in a medium under conditions permissive for the production of glycosylated product,
optionally separating glycosylated product from the culture.

28. Method for the production of glycosylated product by a genetically modified yeast cell, comprising the steps of:
providing a yeast cell genetically modified for the production of glycosylated product, the cell comprising at least one nucleic acid sequence coding for an enzyme for glycosylated product synthesis,
the cell further genetically modified for i) modified expression of cell wall mannosylated protein, ii) modified expression of beta1,3 glucan, iii) modified expression of beta 1,6 glucan, and/or iv) modified expression of chitin providing reduced cell wall biosynthesis,
culturing the cell in a medium under conditions permissive for the production of glycosylated product,
optionally separating glycosylated product from the culture.

29. Method for the production of glycosylated product by a genetically modified Corynebacterium, Nocardia or Mycobacterium cell, comprising the steps of:
providing a Corynebacterium, Nocardia or Mycobacterium cell genetically modified for the production of glycosylated product, the cell comprising at least one nucleic acid sequence coding for an enzyme for glycosylated product synthesis,
the cell further genetically modified for i) modified expression of mycolic acid biosynthesis, or ii) modified expression of arabinogalactan biosynthesis providing reduced cell wall biosynthesis,
culturing the cell in a medium under conditions permissive for the production of glycosylated product,
optionally separating glycosylated product from the culture.

30. Method for the production of glycosylated product by a genetically modified Bacillus cell, comprising the steps of:
providing a cell genetically modified for the production of glycosylated product, the cell comprising at least one nucleic acid sequence coding for an enzyme for glycosylated product synthesis,
the cell further genetically modified for modified expression of teichoic acid biosynthesis providing reduced cell wall biosynthesis,
culturing the cell in a medium under conditions permissive for the production of glycosylated product,
optionally separating glycosylated product from the culture.

31. A method for the production of glycosylated product, the method comprising the steps of:
a) providing a cell of a micro-organism according to any one of embodiments 1 to 20,
b) culturing the cell in a medium under conditions permissive for the production of the glycosylated product,
c) optionally separating the glycosylated product from the culture.

32. Method according to any one of embodiment 21 to 31, the cell wall biosynthesis is reduced by deletion, reduced expression or abolished expression of at least one glycosyltransferase within the cell wall biosynthesis pathway.

33. Method according to any one of embodiment 22 to 32, wherein the glycosylated product is chosen from saccharide, a glycosylated aglycon, a glycolipid or a glycoprotein.

34. Method according to any one of embodiment 22 to 33, wherein the glycosylated product is an oligosaccharide, preferably a mammalian milk oligosaccharide.

35. Method according to any one of embodiment 22 to 34, wherein the glycosylated product is an oligosaccharide, preferably an oligosaccharide with a degree of polymerization higher than 3.

36. Method according to any one of embodiment 27 to 35, wherein the reduced cell wall biosynthesis is obtained by modified expression, wherein the modified expression is obtained by deletion, reduced expression or abolished expression.

37. Use of a micro-organism according to any one of the embodiments 1 to 20, in a method for the production of an oligosaccharide, preferably a mammalian milk oligosaccharide.

38. Method according to embodiment 27, characterized in that the cell is an Escherichia coli cell.

39. Method for the production of glycosylated product by a genetically modified cell in a bioreactor, comprising the steps of:
providing a cell genetically modified for the production of glycosylated product, the cell comprising at least one nucleic acid sequence coding for an enzyme for glycosylated product synthesis,
culturing the cell in a medium under conditions permissive for the production of glycosylated product,

characterized in that the vessel filling of the bioreactor is equal to or higher than 50%.

40. Method according to embodiment 39, wherein the cell is a cell of a micro-organism according to any one of embodiments 1 to 20.

41. Method according to any one of embodiment 39 or 40, wherein the glycosylated product is an oligosaccharide, preferably a mammalian milk oligosaccharide, more preferably chosen from the group of fucosylated oligosaccharide, neutral oligosaccharide or sialylated oligosaccharide, most preferably chosen from 2′-fucosyllactose, 3-fucosyllactose, difucosyllactose, Lacto-N-tetraose, Lacto-N-neotetraose, 3′-sialyllactose, 6′-sialyllactose, lacto-N-fucopentaose II, lacto-N-fucopentaose I, lacto-N-fucopentaose III, lacto-N-fucopentaose V, lacto-N-fucopentaose VI, sialyllacto-N-tetraose c (LSTc), sialyllacto-N-tetraose b (LSTb), sialyllacto-N-tetraose a (LSTa).

Moreover, the disclosure relates to the following preferred specific embodiments:

1. A micro-organism genetically modified for the production of at least one glycosylated product characterized in that the micro-organism has a cell wall biosynthesis that is reduced by a deletion, reduced or abolished expression of at least one enzyme within the cell wall biosynthesis pathway,

wherein the micro-organism is a bacterium or yeast, and

wherein the cell wall biosynthesis pathway is at least one pathway chosen from:
cell wall carbohydrate antigen biosynthesis, preferably O-antigen and/or common-antigen biosynthesis when the micro-organism is a Gram-negative bacterium,
capsular polysaccharide biosynthesis,
cell wall protein mannosylation biosynthesis, beta-1,3-glucan biosynthesis, beta-1,6-glucan biosynthesis and/or chitin biosynthesis when the micro-organism is a yeast,
mycolic acid and/or arabinogalactan biosynthesis when the micro-organism is a Corynebacterium, Nocardia or Mycobacterium,
teichoic acid biosynthesis when the micro-organism is a Gram-positive bacterium, preferably Bacillus.

2. Micro-organism according to preferred embodiment 1, wherein the reduced cell wall biosynthesis pathway is combined with the introduction of one or more pathways for the synthesis of one or more nucleotide-activated sugars.

3. Micro-organism according to any one of preferred embodiment 1 or 2, wherein the micro-organism is further modified to express one or more glycosyltransferases for production of the glycosylated product.

4. Micro-organism according to any one of preferred embodiment 1 to 3, wherein the glycosylated product is an oligosaccharide, a glycosylated aglycon, a glycolipid or a glycoprotein.

5. Micro-organism according to any one of preferred embodiment 1 to 4, wherein the enzyme within the cell wall biosynthesis pathway is a glycosyltransferase.

6. Micro-organism according to any one of preferred embodiments 1 to 5, wherein the micro-organism is a bacterium chosen from Escherichia, Bacillus, Lactobacillus, Lactococcus, Corynebacterium.

7. Micro-organism according to any one of preferred embodiments 1 to 5, wherein the micro-organism is a yeast chosen from Pichia, Hansenula, Komagataella, Saccharomyces.

8. Micro-organism according to any one of preferred embodiments 1 to 6, wherein the micro-organism is a bacterium with a further cell wall biosynthesis pathway that is reduced by a deletion, reduced or abolished expression of at least one enzyme within the further cell wall biosynthesis pathway chosen from colanic acid biosynthesis, exopolysaccharide biosynthesis and/or lipopolysaccharide biosynthesis.

9. Micro-organism according to any one of preferred embodiments 1 to 6 and 8, wherein the micro-organism is a Gram-negative bacterium having a reduced cell wall biosynthesis by a reduced O-antigen biosynthesis wherein the reduction in the O-antigen biosynthesis is provided by a deletion, reduced or abolished expression of any one or more of the genes present in the O-antigen biosynthesis gene cluster comprising rhamnosyltransferase, putative glycosyltransferase, putative lipopolysaccharide biosynthesis O-acetyl transferase, β-1,6-galactofuranosyltransferase, putative O-antigen polymerase, UDP-galactopyranose mutase, polyisoprenol-linked O-antigen repeat unit flippase, dTDP-4-dehydrorhamnose 3,5-epimerase, dTDP-glucose pyrophosphorylase, dTDP-4-dehydrorhamnose reductase, dTDP-glucose 4,6-dehydratase 1, UTP:glucose-1-phosphate uridylyltransferase.

10. Micro-organism according to preferred embodiment 9, wherein the reduction in the O-antigen biosynthesis is provided by a deletion, reduced or abolished expression of any one or more of i) WbbL, WbbK, WbbJ, WbbI, WbbH, glf, rfbX, rfbC, rfbA, rfbD, rfbB, wcaN, preferably as given by SEQ ID NOs:27 to 38, respectively, or ii) a polypeptide sequence having 80% or more sequence identity to the full-length sequence of any one of the SEQ ID NOs:27 to 38 and having rhamnosyltransferase activity, glycosyltransferase activity, lipopolysaccharide biosynthesis O-acetyl transferase activity, β-1,6-galactofuranosyltransferase activity, O-antigen polymerase activity, UDP-galactopyranose mutase activity, polyisoprenol-linked O-antigen repeat unit flippase activity, dTDP-4-dehydrorhamnose 3,5-epimerase activity, dTDP-glucose pyrophosphorylase activity, dTDP-4-dehydrorhamnose reductase activity, dTDP-glucose 4,6-dehydratase 1 activity or UTP:glucose-1-phosphate uridylyltransferase activity, respectively.

11. Micro-organism according to any one of preferred embodiments 1 to 6 and 8, wherein the micro-organism is a Gram-negative bacterium having a reduced cell wall biosynthesis by a reduced common-antigen biosynthesis wherein the reduction in the common-antigen biosynthesis is provided by a deletion, reduced or abolished expression of any one or more of the genes present in the common-antigen biosynthesis gene cluster comprising UDP-N-acetylglucosamine-undecaprenyl-phosphate N-acetylglucosaminephosphotransferase, enterobacterial common antigen polysaccharide co-polymerase, UDP-N-acetylglucosamine 2-epimerase, UDP-N-acetyl-D-mannosamine dehydrogenase, dTDP-glucose 4,6-dehydratase 2, dTDP-glucose pyrophosphorylase, dTDP-4-amino-4,6-dideoxy-D-galactose acyltransferase, dTDP-4-dehydro-6-deoxy-D-glucose transaminase, lipid III flippase, TDP-N-acetylfucosamine:lipid II N-acetylfucosaminyltransferase, putative enterobacterial common antigen polymerase, UDP-N-acetyl-D-mannosaminuronic acid transferase.

12. Micro-organism according to preferred embodiment 11, wherein the reduction in the common-antigen biosynthesis is provided by a deletion, reduced or abolished expression of any one or more of i) rfe, wzzE, wecB, wecC, rffG, rfH, rffC, wecE, wzxE, wecF, wzyE, rffM, preferably as given by SEQ ID NOs:15 to 26, respectively, or ii) a polypeptide sequence having 80% or more sequence identity to the full-length sequence of any one of the SEQ ID NOs:15 to 26 and having UDP-N-acetylglucosamine-undecaprenyl-phosphate N-acetylglucosaminephosphotransferase activity, enterobacterial common antigen polysaccharide co-polymerase activity, UDP-N-acetylglucosamine 2-epimerase activity, UDP-N-acetyl-D-mannosamine dehydrogenase activity, dTDP-glucose 4,6-dehydratase 2 activity, dTDP-glucose pyrophosphorylase activity, dTDP-4-amino-4,6-dideoxy-D-galactose acyltransferase activity, dTDP-4-dehydro-6-deoxy-D-glucose transaminase activity, lipid III flippase activity, TDP-N-acetylfucosamine:lipid II N-acetylfucosaminyltransferase activity, enterobacterial common antigen polymerase activity or UDP-N-acetyl-D-mannosaminuronic acid transferase activity, respectively.

13. Micro-organism according to preferred embodiment 8, wherein the micro-organism is a bacterium having a further reduced cell wall biosynthesis by a reduced colanic acid biosynthesis wherein the reduction in the colanic acid biosynthesis is provided by a deletion, reduced or abolished expression of any one or more of the genes present in the colanic acid biosynthesis gene cluster comprising putative colanic acid biosynthesis protein, putative colanic biosynthesis glycosyl transferase, putative colanic acid biosynthesis pyruvyl transferase, M-antigen undecaprenyl diphosphate flippase, UDP-glucose:undecaprenyl-phosphate glucose-1-phosphate transferase, phosphomannomutase, mannose-1-phosphate guanylyltransferase, colanic acid biosynthesis fucosyltransferase, GDP-mannose mannosyl hydrolase, GDP-L-fucose synthase, GDP-mannose 4,6-dehydratase, colanic acid biosynthesis acetyltransferase, colanic acid biosynthesis fucosyltransferase, putative colanic acid polymerase, colanic acid biosynthesis galactosyltransferase, colanic acid biosynthesis acetyltransferase, colanic acid biosynthesis glucuronosyltransferase, protein-tyrosine kinase, protein-tyrosine phosphatase, outer membrane polysaccharide export protein.

14. Micro-organism according to preferred embodiment 13, wherein the reduction in the colanic acid biosynthesis is provided by a deletion, reduced or abolished expression of any one or more of i) WcaM, WcaL, WcaK, WzxC, wcaJ, cpsG, cpsB, WcaI, gmm, fcl, gmd, WcaF, WcaE, WcaD, WcaC, WcaB, WcaA, Wzc, wzb, Wza, preferably as given by SEQ ID NOs:39 to 58, respectively, or ii) a polypeptide sequence having 80% or more sequence identity to the full-length sequence of any one of the SEQ ID NOs:39 to 58 and having colanic acid biosynthesis protein activity, colanic biosynthesis glycosyl transferase activity, colanic acid biosynthesis pyruvyl transferase activity, M-antigen undecaprenyl diphosphate flippase activity, UDP-glucose:undecaprenyl-phosphate glucose-1-phosphate transferase activity, phosphomannomutase activity, mannose-1-phosphate guanylyltransferase activity, colanic acid biosynthesis fucosyltransferase activity, GDP-mannose mannosyl hydrolase activity, GDP-L-fucose synthase activity, GDP-mannose 4,6-dehydratase activity, colanic acid biosynthesis acetyltransferase activity, colanic acid biosynthesis fucosyltransferase activity, colanic acid polymerase activity, colanic acid biosynthesis galactosyltransferase activity, colanic acid biosynthesis acetyltransferase activity, colanic acid biosynthesis glucuronosyltransferase activity, protein-tyrosine kinase activity, protein-tyrosine phosphatase activity or outer membrane polysaccharide export protein activity, respectively.

15. Micro-organism according to any one of preferred embodiments 1 to 5 and 7, wherein the micro-organism is a yeast having a reduced cell wall biosynthesis by a reduced cell wall protein mannosylation biosynthesis wherein the reduction of the cell wall protein mannosylation biosynthesis is provided by a deletion, reduced or abolished expression of any one or more of Protein-O-mannosyltransferase encoding gene preferably one or more of PMT1, PMT2, PMT3, PMT4, PMT5, PMT6, PMT7, more preferably one or more of PMT1, PMT2, PMT4.

16. Micro-organism according to any one of preferred embodiments 1 to 6 and 8, wherein the micro-organism is a Corynebacterium, Nocardia or Mycobacterium having a reduced cell wall biosynthesis by a reduced mycolic acid and/or arabinogalactan biosynthesis wherein the reduced mycolic acid and/or arabinogalactan biosynthesis is provided by a reduced expression of any one or more of mycolic acid and/or arabinogalactan biosynthesis genes, preferably by reduced expression of any one or more of accD2, accD3, aftA, aftB or emb.

17. Micro-organism according to any one of preferred embodiments 1 to 6 and 8, wherein the micro-organism is a Gram-positive bacterium having a reduced cell wall biosynthesis by a reduced teichoic acid biosynthesis wherein the reduced teichoic acid biosynthesis is provided by a reduced expression of any one or more of teichoic acid biosynthesis genes, preferably by reduced expression of any one or more of tagO, tagA, tagB, tagD, tagF, tagG or tagH.

18. Micro-organism according to any one of preferred embodiments 1 to 17, wherein the glycosylated product is an oligosaccharide with a degree of polymerization higher than 3.

19. Isolated micro-organism according to any one of preferred embodiments 1 to 18.

20. A method to reduce the viscosity, foaming, and/or airlift of a fermentation process with a micro-organism characterized in that the cell wall biosynthesis of the micro-organism is reduced by deletion, reduced or abolished expression of at least one enzyme within the cell wall biosynthesis pathway,

wherein the micro-organism is a bacterium or yeast, and

wherein the cell wall biosynthesis pathway is at least one pathway chosen from:
cell wall carbohydrate antigen biosynthesis, preferably O-antigen and/or common-antigen biosynthesis when the micro-organism is a Gram-negative bacterium,
capsular polysaccharide biosynthesis,
cell wall protein mannosylation biosynthesis, beta-1,3-glucan biosynthesis, beta-1,6-glucan biosynthesis and/or chitin biosynthesis when the micro-organism is a yeast,
mycolic acid and/or arabinogalactan biosynthesis when the micro-organism is a Corynebacterium, Nocardia or Mycobacterium,
teichoic acid biosynthesis when the micro-organism is a Gram-positive bacterium, preferably Bacillus.

21. Method according to preferred embodiment 20, wherein the micro-organism is further modified to produce at least one glycosylated product.

22. Method for the production of glycosylated product by a genetically modified cell, comprising the steps of:
providing a cell genetically modified for the production of glycosylated product, the cell comprising at least one nucleic acid sequence coding for an enzyme for glycosylated product synthesis,
the cell further genetically modified for reduced cell wall biosynthesis by deletion, reduced or abolished expression of at least one enzyme within the cell wall biosynthesis pathway, wherein the cell wall biosynthesis pathway is at least one pathway chosen from cell wall carbohydrate antigen biosynthesis, capsular polysaccharide biosynthesis, cell wall protein mannosylation biosynthesis, beta-1,3-glucan biosynthesis, beta-1,6-glucan biosynthesis, chitin biosynthesis, mycolic acid biosynthesis, arabinogalactan biosynthesis and teichoic acid biosynthesis, preferably wherein the cell wall carbohydrate antigen biosynthesis is O-antigen and/or common-antigen biosynthesis,
culturing the cell in a medium under conditions permissive for the production of glycosylated product,
optionally separating glycosylated product from the culture.

23. Method according to preferred embodiment 22, wherein the enzyme for glycosylated product synthesis comprises enzymes involved in nucleotide-activated sugar synthesis and glycosyltransferases.

24. Method according to any one of preferred embodiment 22 or 23, wherein the genetically modified cell is a micro-organism, preferably bacterium or yeast.

25. Method according to any one of preferred embodiment 22 to 24, wherein the genetically modified cell is a bacterium, preferably Enterobacteriaceae, more preferably Escherichia.

26. Method according to any one of preferred embodiment 22 to 24, wherein the genetically modified cell is a yeast, preferably Pichia, Hansenula, Komagataella, Saccharomyces.

27. Method for the production of glycosylated product by a genetically modified Gram-negative bacterial cell, comprising the steps of:
providing a Gram-negative bacterial cell genetically modified for the production of glycosylated product, the cell comprising at least one nucleic acid sequence coding for an enzyme for glycosylated product synthesis,
the cell further genetically modified for reduced cell wall biosynthesis by deletion, reduced or abolished expression of at least one enzyme within the cell wall biosynthesis pathway, the cell wall biosynthesis being cell wall carbohydrate antigen biosynthesis, preferably O-antigen and/or common-antigen biosynthesis,
culturing the cell in a medium under conditions permissive for the production of glycosylated product,
optionally separating glycosylated product from the culture.

28. Method according to preferred embodiment 27, wherein the enzyme for glycosylated product synthesis comprises enzymes involved in nucleotide-activated sugar synthesis and glycosyltransferases.

29. Method according to any one of preferred embodiment 27 or 28, wherein the Gram-negative bacterial cell has a further cell wall biosynthesis pathway that is reduced by a deletion, reduced or abolished expression of at least one enzyme within the further cell wall biosynthesis pathway chosen from colanic acid biosynthesis, exopolysaccharide biosynthesis and/or lipopolysaccharide biosynthesis.

30. Method for the production of glycosylated product by a genetically modified yeast cell, comprising the steps of:
providing a yeast cell genetically modified for the production of glycosylated product, the cell comprising at least one nucleic acid sequence coding for an enzyme for glycosylated product synthesis,
the cell further genetically modified for reduced cell wall biosynthesis by deletion, reduced or abolished expression of at least one enzyme within the cell wall biosynthesis pathway, the cell wall biosynthesis being i) cell wall protein mannosylation biosynthesis, ii) beta-1,3-glucan biosynthesis, iii) beta-1,6-glucan biosynthesis, and/or iv) chitin biosynthesis,
culturing the cell in a medium under conditions permissive for the production of glycosylated product,
optionally separating glycosylated product from the culture.

31. Method according to preferred embodiment 30, wherein the enzyme for glycosylated product synthesis comprises enzymes involved in nucleotide-activated sugar synthesis and glycosyltransferases.

32. Method for the production of glycosylated product by a genetically modified Corynebacterium, Nocardia or Mycobacterium cell, comprising the steps of:
providing a Corynebacterium, Nocardia or Mycobacterium cell genetically modified for the production of glycosylated product, the cell comprising at least one nucleic acid sequence coding for an enzyme for glycosylated product synthesis,
the cell further genetically modified for reduced cell wall biosynthesis by deletion, reduced or abolished expression of at least one enzyme within the cell wall biosynthesis pathway, the cell wall biosynthesis being i) mycolic acid biosynthesis, and/or ii) arabinogalactan biosynthesis,
culturing the cell in a medium under conditions permissive for the production of glycosylated product,
optionally separating glycosylated product from the culture.

33. Method according to preferred embodiment 32, wherein the enzyme for glycosylated product synthesis comprises enzymes involved in nucleotide-activated sugar synthesis and glycosyltransferases.

34. Method for the production of glycosylated product by a genetically modified Bacillus cell, comprising the steps of:
providing a Bacillus cell genetically modified for the production of glycosylated product, the cell comprising at least one nucleic acid sequence coding for an enzyme for glycosylated product synthesis,
the cell further genetically modified for reduced cell wall biosynthesis by deletion, reduced or abolished expression of at least one enzyme within the cell wall biosynthesis pathway, the cell wall biosynthesis being teichoic acid biosynthesis,
culturing the cell in a medium under conditions permissive for the production of glycosylated product,
optionally separating glycosylated product from the culture.

35. Method according preferred embodiment 34, wherein the enzyme for glycosylated product synthesis comprises enzymes involved in nucleotide-activated sugar synthesis and glycosyltransferases.

36. A method for the production of glycosylated product, the method comprising the steps of:
a) providing a cell of a micro-organism according to any one of preferred embodiments 1 to 19,
b) culturing the cell in a medium under conditions permissive for the production of the glycosylated product,
c) optionally separating the glycosylated product from the culture.

37. Method according to any one of preferred embodiments 20 to 36, wherein the cell wall biosynthesis is reduced by deletion, reduced or abolished expression of at least one glycosyltransferase within the cell wall biosynthesis pathway.

38. Method according to any one of preferred embodiments 20 to 37, wherein the glycosylated product is chosen from saccharide, a glycosylated aglycon, a glycolipid or a glycoprotein.

39. Method according to any one of preferred embodiments 20 to 38, wherein the glycosylated product is an oligosaccharide, preferably a mammalian milk oligosaccharide.

40. Method according to any one of preferred embodiments 20 to 39, wherein the glycosylated product is an oligosaccharide, preferably an oligosaccharide with a degree of polymerization higher than 3.

41. Use of a micro-organism according to any one of the preferred embodiments 1 to 19, in a method for the production of an oligosaccharide, preferably a mammalian milk oligosaccharide.

42. Method according to preferred embodiment 27, characterized in that the cell is an Escherichia coli cell.

43. Method for the production of glycosylated product by a genetically modified cell in a bioreactor, comprising the steps of:
providing a cell genetically modified for the production of glycosylated product, the cell comprising at least one nucleic acid sequence coding for an enzyme for glycosylated product synthesis,
culturing the cell in a medium under conditions permissive for the production of glycosylated product,

characterized in that the vessel filling of the bioreactor is equal to or higher than 50%.

44. Method according to preferred embodiment 43, wherein the enzyme for glycosylated product synthesis comprises enzymes involved in nucleotide-activated sugar synthesis and glycosyltransferases.

45. Method according to any one of preferred embodiment 43 or 44, wherein the cell is a cell of a micro-organism according to any one of preferred embodiments 1 to 19.

46. Method according to any one of preferred embodiment 43 to 45, wherein the glycosylated product is an oligosaccharide, preferably a mammalian milk oligosaccharide, more preferably chosen from the group of fucosylated oligosaccharide, neutral oligosaccharide or sialylated oligosaccharide, most preferably chosen from 2′-fucosyllactose, 3-fucosyllactose, difucosyllactose, Lacto-N-tetraose, Lacto-N-neotetraose, 3′-sialyllactose, 6′-sialyllactose, lacto-N-fucopentaose II, lacto-N-fucopentaose I, lacto-N-fucopentaose III, lacto-N-fucopentaose V, lacto-N-fucopentaose VI, sialyllacto-N-tetraose d (LSTd), sialyllacto-N-tetraose c (LSTc), sialyllacto-N-tetraose b (LSTb), sialyllacto-N-tetraose a (LSTa).

The following examples will serve as further illustration and clarification of the disclosure and are not intended to be limiting.

### EXAMPLES

### Example 1: Material and Methods

Material and Methods Escherichia coli

Media

Three different media were used, namely a rich Luria Broth (LB), a minimal medium for shake flask (MMsf) and a minimal medium for fermentation (MMf). Both minimal media use a trace element mix.

Trace element mix consisted of 3.6 g/L FeCl₂.4H₂O, 5 g/L CaCl₂.2H₂O, 1.3 g/L MnCl₂.2H₂O, 0.38 g/L CuCl₂.2H₂O, 0.5 g/L CoCl₂.6H₂O, 0.94 g/L ZnCl₂, 0.0311 g/L H₃BO₄, 0.4 g/L Na2EDTA.2H₂O and 1.01 g/L thiamine.HCl. The molybdate solution contained 0.967 g/L NaMoO₄.2H₂O. The selenium solution contained 42 g/L SeO₂.

The Luria Broth (LB) medium consisted of 1% tryptone peptone (Difco, Erembodegem, Belgium), 0.5% yeast extract (Difco) and 0.5% sodium chloride (VWR. Leuven, Belgium). Luria Broth agar (LBA) plates consisted of the LB media, with 12 g/L agar (Difco, Erembodegem, Belgium) added.

The minimal medium for the shake flasks (MMsf) experiments contained 2.00 g/L NH₄Cl, 5.00 g/L (NH₄)2SO₄, 2.993 g/L KH₂PO₄, 7.315 g/L K₂HPO₄, 8.372 g/L MOPS, 0.5 g/L NaCl, 0.5 g/L MgSO₄.7H₂O, 14.26 g/L sucrose or another carbon source when specified in the examples, 1 ml/L trace element mix, 100 μl/L molybdate solution, and 1 mL/L selenium solution.

The medium was set to a pH of 7 with 1M KOH. Depending on the experiment lactose, LNB or LacNAc could be added as a precursor.

The minimal medium for fermentations (MMf) contained 6.75 g/L NH₄Cl, 1.25 g/L (NH₄)2SO₄, 2.93 g/L KH₂PO₄ and 7.31 g/L KH₂PO₄, 0.5 g/L NaCl, 0.5 g/L MgSO₄.7H₂O, 14.26 g/L sucrose, 1 mL/L trace element mix, 100 μL/L molybdate solution, and 1 mL/L selenium solution with the same composition as described above.

Complex medium, e.g., LB, was sterilized by autoclaving (121° C., 21′) and minimal medium by filtration (0.22 μm Sartorius). When necessary, the medium was made selective by adding an antibiotic (e.g., ampicillin (100 mg/L), chloramphenicol (20 mg/L), carbenicillin (100 mg/L), spectinomycin (40 mg/L) and/or kanamycin (50 mg/L)).

Plasmids

pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, Belgium in 2007).

Plasmids were maintained in the host E coli DH5alpha (F⁻, phi80dlacZAM15, Δ(lacZYA-argF) U169, deoR, recAl, endAl, hsdR17(rk⁻, mk⁺), phoA, supE44, lambda⁻, thi-1, gyrA96, relAI) bought from Invitrogen.

Strains and Mutations

Escherichia coli K12 MG1655 [λ⁻, F⁻, rph-1] was obtained from the Coli Genetic Stock Center (US), CGSC Strain#: 7740, in March 2007. Gene disruptions, gene introductions and gene replacements were performed using the technique published by Datsenko and Wanner (PNAS 97 (2000), 6640-6645). This technique is based on antibiotic selection after homologous recombination performed by lambda Red recombinase. Subsequent catalysis of a flippase recombinase ensures removal of the antibiotic selection cassette in the final production strain.

Transformants carrying a Red helper plasmid pKD46 were grown in 10 ml LB media with ampicillin, (100 mg/L) and L-arabinose (10 mM) at 30° C. to an OD₆₀₀ nm of 0.6. The cells were made electrocompetent by washing them with 50 ml of ice-cold water, a first time, and with 1 ml ice cold water, a second time. Then, the cells were resuspended in 50 μl of ice-cold water. Electroporation was done with 50 μl of cells and 10-100 ng of linear double-stranded-DNA product by using a Gene Pulser™ (BioRad) (600 Ω, 25 μFD, and 250 volts).

After electroporation, cells were added to 1 ml LB media incubated 1 h at 37° C., and finally spread onto LB-agar containing 25 mg/L of chloramphenicol or 50 mg/L of kanamycin to select antibiotic resistant transformants. The selected mutants were verified by PCR with primers upstream and downstream of the modified region and were grown in LB-agar at 42° C. for the loss of the helper plasmid. The mutants were tested for ampicillin sensitivity.

The linear ds-DNA amplicons were obtained by PCR using pKD3, pKD4 and their derivates as template. The primers used had a part of the sequence complementary to the template and another part complementary to the side on the chromosomal DNA where the recombination must take place. For the genomic knock-out, the region of homology was designed 50-nt upstream and 50-nt downstream of the start and stop codon of the gene of interest. For the genomic knock-in, the transcriptional starting point (+1) had to be respected. PCR products were PCR-purified, digested with Dpnl, repurified from an agarose gel, and suspended in elution buffer (5 mM Tris, pH 8.0).

The selected mutants (chloramphenicol or kanamycin resistant) were transformed with pCP20 plasmid, which is an ampicillin and chloramphenicol resistant plasmid that shows temperature-sensitive replication and thermal induction of FLP synthesis. The ampicillin-resistant transformants were selected at 30° C., after which a few were colony purified in LB at 42° C. and then tested for loss of all antibiotic resistance and of the FLP helper plasmid. The gene knock outs and knock ins are checked with control primers (Fw/Rv-gene-out).

For 2′FL, 3FL and diFL production, the mutant strains derived from E. coli K12 MG1655 have knock-outs of the genes lacZ, lacY, lacA, glgC, agp, pfkA, pfkB, pgi, arcA, iciR, wcaJ, pgi, Ion and hyA and additionally genomic knock-ins of constitutive expression constructs containing the E coli lacY gene, a fructose kinase gene (frk) originating from Zymomonas mobilis and a sucrose phosphorylase (SP) originating from Bifidobacterium adolescentis. These genetic modifications are also described in WO2016075243 and WO2012007481. In addition, an alpha-1,2- and/or alpha-1,3-fucosyltransferase expression plasmid is added to the strains.

For LNT and LNnT production, the strain has a genomic knock out of the lacZ gene and nagB gene and knock-ins of constitutive expression constructs containing a galactoside beta-1,3-N-acetylglucosaminyltransferase (lgtA) from Neisseria meningitidis (SEQ ID NO:3) and either an N-acetylglucosamide beta-1,3-galactosyltransferase (wbgO) from Escherichia coli O55:H₇ (SEQ ID NO:4) for LNT production or an N-acetylglucosamide beta-1,4-galactosyltransferase (lgtB) from Neisseria meningitidis (SEQ ID NO:5) for LNnT production.

For 3′SL and 6′SL production the strains are described in WO18122225. The mutant strain has the following gene knock-outs: lacZ, nagABCDE, nanA, nanE, nanK, manXYZ. Additionally, the strain has genomic knock-ins of constitutive expression constructs containing a mutated variant of the L-glutamine-D-fructose-6-phosphate aminotransferase (gImS) from Escherichia coli (SEQ ID NO:6), a glucosamine 6-phosphate N-acetyltransferase (GNA1) from Saccharomyces cerevisiae (SEQ ID NO:7), an N-acetylglucosamine 2-epimerase (BoAGE) from Bacteroides ovatus (SEQ ID NO:8), an N-acetylneuraminate synthase (NeuB) from Campylobacter jejuni (SEQ ID NO: CMP-Neu5Ac synthetase (NeuA) from Campylobacter jejuni (SEQ ID NO:10), and either a beta-galactoside alpha-2,3-sialyltransferase from Pasteurella multocida (SEQ ID NO:11) for 3′SL production or a beta-galactoside alpha-2,6-sialyltransferase from Photobacterium damselae (SEQ ID NO:12) for 6′SL production.

All constitutive promoters and UTRs originate from the libraries described by De Mey et al. (BMC Biotechnology, 2007) and Mutalik et al. (Nat. Methods 2013, No. 10, 354-360). All genes were ordered synthetically at Twist Bioscience (twistbioscience.com) or IDT (eu.idtdna.com) and the codon usage was adapted using the tools of the supplier.

All strains are stored in cryovials at −80° C. (overnight LB culture mixed in a 1:1 ratio with 70% glycerol).

Cultivation Conditions

A preculture of 96well microtiter plate experiments was started from a cryovial, in 150 μL LB and was incubated overnight at 37° C. on an orbital shaker at 800 rpm. This culture was used as inoculum for a 96well square microtiter plate, with 400 μL MMsf medium by diluting 400×. These final 96-well culture plates were then incubated at 37° C. on an orbital shaker at 800 rpm for 72h, or shorter, or longer. At the end of the cultivation experiment samples were taken from each well to measure sugar concentrations in the broth supernatant (extracellular sugar concentrations, after spinning down the cells), or by boiling the culture broth for 15 min at 90° C. or 60 min at 60° C. before spinning down the cells (=whole broth measurements, average of intra- and extracellular sugar concentrations).

Also, a dilution of the cultures was made to measure the optical density at 600 nm. The cell performance index or CPI is determined by dividing the oligosaccharide concentrations by the biomass, in relative percentages compared to a reference strain. The biomass is empirically determined to be approximately ⅓^{rd} of the optical density measured at 600 nm. The oligosaccharide export ratio was determined by dividing the oligosaccharide concentrations measured in the supernatant by the oligosaccharide concentrations measured in the whole broth, in relative percentages compared to a reference strain.

A preculture for the bioreactor was started from an entire 1 mL cryovial of a certain strain, inoculated in 250 mL or 500 mL of MMsf medium in a 1 L or 2.5 L shake flask and incubated for 24 h at 37° C. on an orbital shaker at 200 rpm. A 5 L bioreactor (having 5 L working volume) (BIOSTAT® B-CDU) was then inoculated (250 mL inoculum in 2 L batch medium); the process was controlled by MFCS control software (Sartorius Stedim Biotech, Melsungen, Germany). Culturing condition were set to 37° C., and maximal stirring; pressure gas flow rates were dependent on the strain and bioreactor. The pH was controlled at 6.8 using 0.5 M H₂SO₄ and 20% NH4OH. The exhaust gas was cooled. 10% solution of silicone antifoaming agent was added when foaming raised during the fermentation.

Material and methods Bacillus subtilis

Media

Two different media are used, namely a rich Luria Broth (LB) and a minimal medium for shake flask (MMsf). The minimal medium uses a trace element mix.

Trace element mix consisted of 0.735 g/L CaCl2.2H2O, 0.1 g/L MnCl2.2H2O, 0.033 g/L CuCl2.2H2O, 0.06 g/L CoCl2.6H2O, 0.17 g/L ZnCl2, 0.0311 g/L H3BO4, 0.4 g/L Na2EDTA.2H2O and 0.06 g/L Na2MoO4. The Fe-citrate solution contained 0.135 g/L FeCl3.6H2O, 1 g/L Na-citrate (Hoch 1973 PMC1212887).

The Luria Broth (LB) medium consisted of 1% tryptone peptone (Difco, Erembodegem, Belgium), 0.5% yeast extract (Difco) and 0.5% sodium chloride (VWR. Leuven, Belgium). Luria Broth agar (LBA) plates consisted of the LB media, with 12 g/L agar (Difco, Erembodegem, Belgium) added.

The minimal medium for the shake flasks (MMsf) experiments contained 2.00 g/L (NH₄)2SO₄, 7.5 g/L KH₂PO₄, 17.5 g/L K₂HPO₄, 1.25 g/L Na-citrate, 0.25 g/L MgSO₄.7H₂O, 0.05 g/L tryptophan, from 10 up to 30 g/L glucose or another carbon source including but not limited to fructose, maltose, sucrose, glycerol and maltotriose when specified in the examples, 10 ml/L trace element mix and 10 ml/L Fe-citrate solution. The medium was set to a pH of 7 with 1M KOH. Depending on the experiment lactose, LNB or LacNAc could be added as a precursor.

Complex medium, e.g., LB, was sterilized by autoclaving (121° C., 21′) and minimal medium by filtration (0.22 μm Sartorius). When necessary, the medium was made selective by adding an antibiotic (e.g., zeocin (20 mg/L)).

Strains, Plasmids and Mutations

Bacillus subtilis 168, available at Bacillus Genetic Stock Center (Ohio, USA).

Plasmids for gene deletion via Cre/lox are constructed as described by Yan et al. (Appl. & Environm. Microbial., Sep. 2008, p5556-5562). Gene disruption is done via homologous recombination with linear DNA and transformation via electroporation as described by Xue et al. (J. Microb. Meth. 34 (1999) 183-191). The method of gene knockouts is described by Liu et al. (Metab. Engine. 24 (2014) 61-69). This method uses 1000 bp homologies up- and downstream of the target gene.

Integrative vectors as described by Popp et al. (Sci. Rep., 2017, 7, 15158) are used as expression vector and could be further used for genomic integrations if necessary. A suitable promoter for expression can be derived from the part repository (iGem): sequence id: Bba_K143012, Bba_K823000, Bba_K823002 or Bba_K823003. Cloning can be performed using Gibson Assembly, Golden Gate assembly, Cliva assembly, LCR or restriction ligation.

For the production of lactose-based oligosaccharides, Bacillus subtilis mutant strains are created to contain a gene coding for a lactose importer (such as the E. coli lacY gene). For 2′FL, 3FL and diFL production, an alpha-1,2- and/or alpha-1,3-fucosyltransferase expression construct is additionally added to the strains. For LNT and LNnT production, expression constructs are added that code for a galactoside beta-1,3-N-acetylglucosaminyltransferase (lgtA) from Neisseria meningitidis and either an N-acetylglucosamide beta-1,3-galactosyltransferase (wbgO) from Escherichia coli O55:H₇ for LNT production or an N-acetylglucosamide beta-1,4-galactosyltransferase (lgtB) from Neisseria meningitidis for LNnT production. For 3′-SL and 6′-SL production, the strains are described in WO18122225. A sialic acid producing B. subtilis strain is obtained by overexpressing the native fructose-6-P-aminotransferase (BsglmS) to enhance the intracellular glucosamine-6-phosphate pool. Further on, the enzymatic activities of the genes nagA, nagB and gamA were disrupted by genetic knockouts and a glucosamine-6-P-aminotransferase from S. cerevisiae (ScGNA1), an N-acetylglucosamine-2-epimerase from Bacteroides ovatus (BoAGE) and a sialic acid synthase from Campylobacter jejuni (CjneuB) were overexpressed on the genome. To allow production of 6′-SL, a CMP-sialic acid synthetase from Neisseria meningitidis (NmneuA) and a sialyltransferase from Photobacterium damselae (PdbST) were overexpressed. To allow production of 3′-SL, a CMP-sialic acid synthetase from Neisseria meningitidis (NmneuA) and a sialyltransferase from Neisseria meningitidis (NmST) were overexpressed.

Heterologous and Homologous Expression

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

Cultivation Conditions

A preculture of 96well microtiter plate experiments was started from a cryovial or a single colony from an LB plate, in 150 μL LB and was incubated overnight at 37° C. on an orbital shaker at 800 rpm. This culture was used as inoculum for a 96well square microtiter plate, with 400 μL MMsf medium by diluting 400×. Each strain was grown in multiple wells of the 96-well plate as biological replicates. These final 96-well culture plates were then incubated at 37° C. on an orbital shaker at 800 rpm for 72h, or shorter, or longer. At the end of the cultivation experiment samples were taken from each well to measure the supernatant concentration (extracellular sugar concentrations, after 5 min. spinning down the cells), or by boiling the culture broth for 15 min at 90° C. or for 60 min at 60° C. before spinning down the cells (=whole broth concentration, intra- and extracellular sugar concentrations, as defined herein).

Also, a dilution of the cultures was made to measure the optical density at 600 nm. The cell performance index or CPI was determined by dividing the oligosaccharide concentrations by the biomass, in relative percentages compared to a reference strain. The biomass is empirically determined to be approximately ⅓^{rd} of the optical density measured at 600 nm.

Material and Methods Corynebacterium glutamicum

Media

Two different media are used, namely a rich tryptone-yeast extract (TY) medium and a minimal medium for shake flask (MMsf). The minimal medium uses a 1000× stock trace element mix.

Trace element mix consisted of 10 g/L CaCl₂), 10 g/L FeSO₄.7H₂O, 10 g/L MnSO₄.H₂O, 1 g/L ZnSO₄.7H₂O, 0.2 g/L CuSO₄, 0.02 g/L NiCl₂.6H₂O, 0.2 g/L biotin (pH 7.0) and 0.03 g/L protocatechuic acid.

The minimal medium for the shake flasks (MMsf) experiments contained 20 g/L (NH₄)₂SO₄, 5 g/L urea, 1 g/L KH₂PO₄, 1 g/L K₂HPO₄, 0.25 g/L MgSO₄.7H₂O, 42 g/L MOPS, from 10 up to 30 g/L glucose or another carbon source including but not limited to fructose, maltose, sucrose, glycerol and maltotriose when specified in the examples and 1 ml/L trace element mix. Depending on the experiment lactose, LNB or LacNAc could be added as a precursor.

The TY medium consisted of 1.6% tryptone (Difco, Erembodegem, Belgium), 1% yeast extract (Difco) and 0.5% sodium chloride (VWR. Leuven, Belgium). TY agar (TYA) plates consisted of the TY media, with 12 g/L agar (Difco, Erembodegem, Belgium) added.

Complex medium, e.g., TY, was sterilized by autoclaving (121° C., 21′) and minimal medium by filtration (0.22 μm Sartorius). When necessary, the medium was made selective by adding an antibiotic (e.g., kanamycin, ampicillin).

Strains and Mutations

Corynebacterium glutamicum ATCC 13032, available at the American Type Culture Collection.

Integrative plasmid vectors based on the Cre/loxP technique as described by Suzuki et al. (Appl. Microbiol. Biotechnol., 2005 Apr., 67(2):225-33) and temperature-sensitive shuttle vectors as described by Okibe et al. (Journal of Microbiological Methods 85, 2011, 155-163) are constructed for gene deletions, mutations and insertions. Suitable promoters for (heterologous) gene expression can be derived from Yim et al. (Biotechnol. Bioeng., 2013 Nov., 110(11):2959-69). Cloning can be performed using Gibson Assembly, Golden Gate assembly, Cliva assembly, LCR or restriction ligation.

For the production of lactose-based oligosaccharides, C. glutamicum mutant strains are created to contain a gene coding for a lactose importer (such as the E. coli lacY gene). For 2′FL, 3FL and diFL production, an alpha-1,2- and/or alpha-1,3-fucosyltransferase expression construct is additionally added to the strains. For LNT and LNnT production, expression constructs are added that code for a galactoside beta-1,3-N-acetylglucosaminyltransferase (lgtA) from Neisseria meningitidis and either an N-acetylglucosamide beta-1,3-galactosyltransferase (wbgO) from Escherichia coli O55:H7 for LNT production or an N-acetylglucosamide beta-1,4-galactosyltransferase (lgtB) from Neisseria meningitidis for LNnT production. For 3′-SL and 6′-SL production, a sialic acid producing C. glutamicum strain is obtained by overexpressing the native fructose-6-P-aminotransferase (CgglmS) to enhance the intracellular glucosamine-6-phosphate pool. Further on, the enzymatic activities of the genes nagA, nagB and gamA were disrupted by genetic knockouts and a glucosamine-6-P-aminotransferase from S. cerevisiae (ScGNA1), an N-acetylglucosamine-2-epimerase from Bacteroides ovatus (BoAGE) and a sialic acid synthase from Campylobacter jejuni (CjneuB) were overexpressed on the genome. In addition, a lactose permease from E. coli (EclacY) was integrated in the genome to establish lactose uptake.

To allow production of 6′-SL, a CMP-sialic acid synthetase from Neisseria meningitidis (NmneuA) and a sialyltransferase from Photobacterium damselae (PdbST) were overexpressed. To allow production of 3′-SL, a CMP-sialic acid synthetase from Neisseria meningitidis (NmneuA) and a sialyltransferase from Neisseria meningitidis (NmST) were overexpressed.

### Heterologous and Homologous Expression

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

Cultivation Conditions

A preculture of 96well microtiter plate experiments was started from a cryovial or a single colony from a TY plate, in 150 μL TY and was incubated overnight at 37° C. on an orbital shaker at 800 rpm. This culture was used as inoculum for a 96-well square microtiter plate, with 400 μL MMsf medium by diluting 400×. Each strain was grown in multiple wells of the 96-well plate as biological replicates. These final 96-well culture plates were then incubated at 37° C. on an orbital shaker at 800 rpm for 72h, or shorter, or longer. At the end of the cultivation experiment samples were taken from each well to measure the supernatant concentration (extracellular sugar concentrations, after 5 min. spinning down the cells), or by boiling the culture broth for 15 min at 60° C. before spinning down the cells (=whole broth concentration, intra- and extracellular sugar concentrations, as defined herein).

Also, a dilution of the cultures was made to measure the optical density at 600 nm. The cell performance index or CPI was determined by dividing the oligosaccharide concentrations, e.g., sialyllactose concentrations, measured in the whole broth by the biomass, in relative percentages compared to the reference strain. The biomass is empirically determined to be approximately ⅓^{rd} of the optical density measured at 600 nm.

Analytical Methods

Optical Density

Cell density of the cultures was frequently monitored by measuring optical density at 600 nm (Implen Nanophotometer NP80, Westburg, Belgium or with a Spark 10M microplate reader, Tecan, Switzerland).

Productivity

The specific productivity Qp is the specific production rate of the oligosaccharide product, typically expressed in mass units of product per mass unit of biomass per time unit (=g oligosaccharide/g biomass/h). The Qp value has been determined for each phase of the fermentation runs, i.e., Batch and Fed-Batch phase, by measuring both the amount of product and biomass formed at the end of each phase and the time frame each phase lasted.

The specific productivity Qs is the specific consumption rate of the substrate, e.g., sucrose, typically expressed in mass units of substrate per mass unit of biomass per time unit (=g sucrose/g biomass/h). The Qs value has been determined for each phase of the fermentation runs, i.e., Batch and Fed-Batch phase, by measuring both the total amount of sucrose consumed and biomass formed at the end of each phase and the time frame each phase lasted.

The yield on sucrose Ys is the fraction of product that is made from substrate and is typically expressed in mass unit of product per mass unit of substrate (=g oligosaccharide/g sucrose). The Ys has been determined for each phase of the fermentation runs, i.e., Batch and Fed-Batch phase, by measuring both the total amount of oligosaccharide produced and total amount of sucrose consumed at the end of each phase.

The yield on biomass Yx is the fraction of biomass that is made from substrate and is typically expressed in mass unit of biomass per mass unit of substrate (=g biomass/g sucrose). The Yp has been determined for each phase of the fermentation runs, i.e., Batch and Fed-Batch phase, by measuring both the total amount of biomass produced and total amount of sucrose consumed at the end of each phase.

The rate is the speed by which the product is made in a fermentation run, typically expressed in concentration of product made per time unit (=g oligosaccharide/L/h). The rate is determined by measuring the concentration of oligosaccharide that has been made at the end of the Fed-Batch phase and dividing this concentration by the total fermentation time.

The lactose conversion rate is the speed by which lactose is consumed in a fermentation run, typically expressed in mass units of lactose per time unit (=g lactose consumed/h). The lactose conversion rate is determined by measurement of the total lactose that is consumed during a fermentation run, divided by the total fermentation time. Similar conversion rates can be calculated for other precursors such as Lacto-N-biose, N-acetyl-lactosamine, Lacto-N-tetraose, or Lacto-N-neotetraose.

Liquid Chromatography

Standards for 2′fucosyllactose, 3-fucosyllactose, difucosyllactose, Lacto-N-tetraose, Lacto-N-neotetraose, 3′sialyllactose and 6′sialyllactose were synthetized in house. Other standards such as but not limited to lactose, sucrose, glucose, fructose were purchased from Sigma, LacNAc and LNB were purchased from Carbosynth.

Carbohydrates were analysed via an UPLC-RI (Waters, USA) method, whereby RI (Refractive Index) detects the change in the refraction index of a mobile phase when containing a sample. All sugars were separated in an isocratic flow using an Acquity UPLC BEH Amide column (Waters, USA) and a mobile phase containing 75 mL acetonitrile, 25 mL Ultrapure water and 0.25 mL triethylamine (for 2′FL, 3FL, DiFL, LNT and LNnT) or containing 70 ml acetonitrile, 26 mL 150 mM ammonium acetate and 4 mL methanol with 0.05% pyrrolidine (for 3′SL and 6′SL). The column size was 2.1×50 mm with 1.7 μm particle size. The temperature of the column was set at 50° C. (for 2′FL, 3FL, DiFL, LNT, LnnT) or 25° C. (for 3′SL and 6′SL) and the pump flow rate was 0.130 mL/min.

Normalization of the Data

For all types of cultivation conditions, data obtained from the mutant strains was normalized against data obtained in identical cultivation conditions with reference strains having an identical genetic background as the mutant strains but lacking the genetic modification of interest. The dashed horizontal line on each plot that is shown in the examples, indicates the setpoint to which all adaptations were normalized. All data is given in relative percentages to that setpoint.

Strain Performance Parameters
oligosaccharide titers (g/L),
production rate r (g oligosaccharide/L/h),
cell performance index CPI (g oligosaccharide/g Biomass),
specific productivity Qp (g oligosaccharide/g Biomass/h),
yield on sucrose Ys (g oligosaccharide/g Sucrose),
sucrose uptake/conversion rate Qs (g Sucrose/g Biomass/h),
lactose conversion/consumption rate rs (g Lactose/h),
oligosaccharide secretion,
growth speed of the production host,
antifoam addition,
viscosity,
airlift,
total fermentation time.

### Example 2: Production of Oligosaccharides in an E. coli Host Lacking Genes for Enterobacterial Common Antigen, O Antigen and/or Colanic Acid Biosynthesis

E. coli mutant strains for the production of oligosaccharides, and more specifically human milk oligosaccharides such as 2′FL, 3FL, 3′SL, 6SL, LNT or LNnT are engineered as described in Example 1. Such strains are further modified to additionally have deletions of all or of a selection of the genes rfe, wzzE, wecB, wecC, rffG, rfEH, rffC, wecE, wzxE, wecF, wzyE or rftM (encoding the proteins of SEQ ID NO:15 to 26), which includes glycosyltransferase-coding genes that are important for the production of the enterobacterial common antigen, a cell surface glycolipid of the E. coli cell wall.

Alternatively, such strains are modified to have deletions of all or of a selection of the genes wbbK, wbbJ, wbbI, wbbH, glf, rfbX, tfbC, rfbA, rfbD, rfbB or wcaN (encoding the proteins of SEQ ID NO:28 to 37 or 38, respectively), which includes glycosyltransferase-coding genes that are important for the production of O-antigen, a polysaccharide structural component of the E. coli cell wall.

Alternatively, such strains are modified to have deletions of all or of a selection of the genes wcaM, wcaL, wcaK, wzxC, wcaJ, cpsG, cpsB, wcaI, gmm, fcl, gmd, wcaF, wcaE, wcaD, wcaC, wcaB, wcaA, wzc, wzb or wza (encoding the proteins of SEQ ID NO:39 to 57 or 58, respectively), which includes glycosyltransferase-coding genes that are important for the production of colanic acid, a negatively charged polysaccharide structural component of the E. coli cell wall. For the production of fucosylated products, when the genes cpsG, cpsB, fcl and gmd (encoding the proteins of SEQ ID NO:44, 45, 48 and 49, respectively) are knocked-out, the production of GDP-fucose should be restored e.g., by adding L-fucose as a substrate and expressing a gene coding for an enzyme having bifunctional fucokinase/L-fucose-1-P-guanylyltransferase activity.

Alternatively, such strains could be further modified to additionally have deletions of multiple of the aforementioned genes that are involved in the biosynthesis of enterobacterial common antigen, O antigen or colanic acid biosynthesis. The resulting mutant strains are thus deficient in multiple of these polysaccharide structural cell wall components.

Any of these aforementioned strains are able to produce any of the listed HMO's, and in similar or potentially higher amounts than the respective reference strains lacking these cell wall structural component deletions. Additionally, the strains grow similarly well or better than their respective reference strains.

These strains can also be evaluated in fed-batch fermentations at bioreactor scale, as described in Example 1. Sucrose can be used as a carbon source and lactose as the precursor for oligosaccharide formation. Examples of other carbon sources are glucose, glycerol, fructose, arabinose, maltotriose, sorbitol, xylose, rhamnose and mannose. The strain's performance in the bioreactor will be similar or better compared to their reference strains in any of the measured parameters listed in Example 1, materials and methods.

### Example 3: Production of 6′SL in a Production Host Lacking Genes for O-Antigen Synthesis

An E. coli mutant strain producing 6′SL as described in Example 1 was used to additionally create a knock-out of the region in the genome encoding the genes wbbK, wbbJ, wbbI, wbbH, glf, rfbX, rfbC, rfbA, rfbD, rfbB and wcaN ((encoding the proteins of SEQ ID NO:28 to 38)). This region includes genes that are important for the production of O-antigen, a polysaccharide structural component of bacterial lipopolysaccharide (LPS), the major component of the outer leaflet of the bacterial membrane. The resulting mutant strain is thus deficient in these polysaccharide structural components.

This strain (“O-antigen KO”) was evaluated and compared to its parent strain not lacking the O-antigen genes (“Reference”) in a growth experiment as described in Example 1. Each strain was grown in 4 multiple wells of a 96-well plate. The dashed horizontal line indicates the setpoint to which all datapoints were normalized.

Table 1 shows the CPI of 6SL of the “O-antigen KO” strain and its maximal growth speed (Mumax), both in relative % normalized to the reference strain (average value f standard deviation). The data indicates that, compared to a reference strain, a higher 6SL CPI is obtained in the strain lacking the genes responsible for O-antigen synthesis, and that its maximal growth speed is slightly increased.

TABLE 1

| | Normalized | Normalized |
|---|---|---|
| | 6SL CPI | Mumax |
| Mutation | (avg % ± sd) | (avg % ± sd) |
| Reference | 100.0 (±4.5) | 100.0 (±7.5) |
| O-antigen KO | 119.2 (±17.8) | 113.6 (±2.9) |

This strain was also evaluated in fed-batch fermentations at bioreactor scale. The bioreactor runs were performed as described in Example 1. Sucrose was used as a carbon source. Lactose was added in the batch medium at 100 g/L as a precursor for 6′SL formation.

The strain's performance in the bioreactor was similar or better compared to the reference strain in all of the parameters listed in Example 1, materials and methods.

### Example 4: Production of 6′SL in a Production Host Lacking Genes for Colanic Acid Synthesis or for O-Antigen and Colanic Acid Synthesis

An E. coli mutant strain producing 6′SL as described in Example 1 was used to additionally create a knock-out of either one or both of the two following regions in the genome. One region includes the genes wcaJ, cpsG, cpsB, wcaI, gmm, fcl and gmd (encoding the proteins of SEQ ID NO:43 to 49), containing glycosyltransferase-coding genes that are important for the production of colanic acid. A second region includes the genes wbbK, wbbJ, wbbI, wbbH, glf, rfbX, frbC, rfbA, rfbD, rfbB, wcaN, wcaM, wcaL, wcaK, wzxC, wcaJ, cpsG, cpsB, wcaI, gmm, fcl, gmd, wcaF, wcaE, wcaD, wcaC, wcaB, wcaA, wzc, wzb and wza (encoding the proteins of SEQ ID NO:28 to 58), containing glycosyltransferase-coding genes important for the production of colanic acid and O-antigen structures on the cell wall. The resulting mutant strains are thus deficient in one or both of these polysaccharide structural components of the cell wall.

These strains (“Colanic acid KO” and “O-antigen and colanic acid KO”) were evaluated and compared to their parent strain not lacking these genes (“Reference”) in a growth experiment as described in Example 1. Each strain was grown in 4 multiple wells of a 96-well plate. The dashed horizontal line indicates the setpoint to which all datapoints were normalized.

Table 2 shows the CPI of 6SL of the “Colanic acid KO” and the “O-antigen and colanic acid KO” strain and their maximal growth speed (Mumax), both in relative % normalized to the reference strain (average value standard deviation). The data indicates that, compared to a reference strain, both a comparable 6SL CPI and maximal growth speed are obtained in the strains lacking genes responsible for either colanic acid or both colanic and O-antigen synthesis.

TABLE 2

| | Normalized 6SL CPI | Normalized Mumax |
|---|---|---|
| Mutation | (avg % ± sd) | (avg % ± sd) |
| Reference | 100 (±7.2) | 100 (±1.7) |
| Colanic acid KO | 92.7 (±6.9) | 98.2 (±2.5) |
| O-antigen and | 97.6 (±3.1) | 96.6 (±3.1) |
| colanic acid KO | | |

### Example 5: Production of 2′FL in a Production Host Lacking Genes for Colanic Acid or Colanic Acid and O-Antigen Synthesis

An E. coli strain was engineered for the production of 2′FL as described in Example 1. Such a strain was further modified to additionally have a knock-out of the region in the genome encoding the genes wbbK, wbbJ, wbbI, wbbH, glf, rfbX, frbC, rfbA, rfbD, rfbB, wcaN, wcaM, wcaL, wcaK, wzxC, wcaJ, cpsG, cpsB, wcaI, gmm, fcl, gmd, wcaF, wcaE, wcaD, wcaC, wcaB, wcaA, wzc, wzb and wza (encoding the proteins of SEQ ID NO:28 to 58), or a knock-out of the region in the genome encompassing the genes wcaM to wza (encoding the proteins of SEQ ID NO:39 to 58) only.

These regions include genes that are important for the production of both colanic acid and O-antigen or colanic acid structures on the cell wall, respectively. The resulting mutant strain is thus deficient in one or both of these polysaccharide structural components.

In addition, the E coli genes encoding for gmd, fcl, cpsG and cpsB (SEQ ID NO:49, 48, 44 and 45, respectively), which are important for the conversion of mannose-6P to GDP-fucose, were cloned using promoters and UTR's as described in Example 1 and expressed in these strains from a plasmid containing a pSC101 ori. More specifically, the four genes were expressed using the following promoters and UTR's from the iGEM BIOFAB collection (http://parts.igem.org/Collections/BioFAB): cpsG using promoter apFAB299 and UTR apFAB890, cpsB using promoter apFAB51 and UTR apFAB896, gmd using promoter apFAB130 and UTR apFAB886 and fcl using promoter apFAB142 and UTR apFAB871. Additionally, a plasmid (pMB1 ori) with a gene coding for an alpha-1,2-fucosyltransferase (HpFutC, (SEQ ID NO:13)) was introduced for the production of 2′FL.

Table 3 shows the CPI of 2′FL of the “Colanic acid KO” and the “O-antigen and colanic acid KO” strains, in relative % normalized to the reference strain (average value standard deviation). The data indicates that 2′FL is clearly produced better in these strains lacking these genes for colanic acid or colanic acid and O-antigen biosynthesis compared to the reference strain.

TABLE 3

| Mutation | Normalized 2'FL CPI (avg % ± sd) |
|---|---|
| Reference | 100 (±0.9) |
| Colanic acid KO | 147.3 (±16.4) |
| O-antigen and | 159.2 (±29.6) |
| colanic acid KO | |

### Example 6: Production of 3FL in a Production Host Lacking Genes for Colanic Acid or Colanic Acid and O-Antigen Synthesis

An E. coli strain was engineered for the production of 3FL as described in Example 1. Such a strain was further modified to additionally have a knock-out of the region in the genome encoding the genes wbbK, wbbJ, wbbI, wbbH, glf, rfbX, frbC, rfbA, rfbD, rfbB, wcaN, wcaM, wcaL, wcaK, wzxC, wcaJ, cpsG, cpsB, wcaI, gmm, fcl, gmd, wcaF, wcaE, wcaD, wcaC, wcaB, wcaA, wzc, wzb and wza ((encoding the proteins of SEQ ID NO:28 to 58)), or a knock-out of the region in the genome encompassing the genes wcaM to wza (encoding the proteins of SEQ ID NO:39 to 58) only.

These regions include genes that are important for the production of both colanic acid and O-antigen or colanic acid structures on the cell wall, respectively. The resulting mutant strain is thus deficient in one or both of these polysaccharide structural components.

In addition, the E coli genes encoding for gmd, fcl, cpsG and cpsB (SEQ ID NO:49, 48, 44 and 45, respectively), which are important for the conversion of mannose-6P to GDP-fucose, were cloned and expressed in these strains from a plasmid containing a pSC101 on as described in example 5. Additionally, a plasmid (pMB1 ori) with a gene coding for an alpha-1,3-fucosyltransferase (3FT, (SEQ ID NO:14)) was introduced for the production of 3FL.

Table 4 shows the CPI of 3FL of the “Colanic acid KO” and the “O-antigen and colanic acid KO” strains, in relative % normalized to the reference strain (average value standard deviation). The data indicates that 3FL production is similar in these strains lacking these genes for colanic acid or colanic acid and O-antigen biosynthesis compared to the reference strain.

TABLE 4

| Mutation | Normalized 3FL CPI (avg % ± sd) |
|---|---|
| Reference | 100 (±7.8) |
| Colanic acid KO | 107.6 (±11.9) |
| O-antigen and | 94.8 (±4.7) |
| colanic acid KO | |

### Example 7: Production of LNT and LNnT in a Production Host Lacking Genes for Colanic Acid and O-Antigen Synthesis

An E coli strain was engineered for the production of LNT or LNnT as described in Example 1. Such a strain was further modified to additionally have a knock-out of the region in the genome encoding all or a selection of the genes wbbL_2, wbbK, wbbJ, wbbI, wbbH, glf, rfbX, frbC, rfbA, rfbD, rfbB, wcaN, wcaM, wcaL, wcaK, wzxC, wcaJ, cpsG, cpsB, wcaI, gmm, fcl, gmd, wcaF, wcaE, wcaD, wcaC, wcaB, wcaA, wzc, wzb and wza (encoding the proteins of SEQ ID NO:27 to 58). More specifically, strains were created with a knock-out of the genes wbbK (encoding the protein of SEQ ID NO:28) or wbbL_2 (encoding the protein of SEQ ID NO:27) to wza (encoding the protein of SEQ ID NO:58), or of the genes wbbK or wbbL_2 (encoding the proteins of SEQ ID NO:28 or 27, respectively) to wcaN (encoding the protein of SEQ ID NO:38) in both a strain producing LNT or LNnT. These regions include genes that are important for the production of both colanic acid and O-antigen, or O-antigen alone, respectively. The resulting mutant strains are thus deficient in one or both of these polysaccharide structural components.

These strains were evaluated and compared to their parent strains not lacking the O-antigen and/or colonic acid genes (“Ref”) in a growth experiment as described in Example 1. Each strain was grown in at least 4 multiple wells of a 96-well plate. The dashed horizontal line indicates the setpoint to which all datapoints were normalized.

Tables 5 and 6 show the CPI of LNT or LNnT and the maximal growth speed (Mumax) of strains lacking important genes of the O-antigen or colanic acid synthesis pathway, or both, in relative % normalized to their reference strains (average value f standard deviation). The data indicates that, compared to a reference strain, a higher CPI is obtained for both LNT or LNnT production in all strains lacking the genes responsible for O-antigen synthesis, or both 0-antigen and colanic acid synthesis.

TABLE 5

| | | Normalized | Normalized |
|---|---|---|---|
| | | LNT CPI | Mumax |
| Mutation | Genes | (avg % ± sd) | (avg % ± sd) |
| Reference | — | 100 (±13.7) | 100 (±12.9) |
| Colanic acid KO | ΔwcaM-wza | 100.8 (±11.5) | 91.7 (±8.5) |
| O-antigen KO | ΔwbbK-wcaN | 132.8 (±7.2) | 104.4 (±0.9) |
| O-antigen KO | ΔwbbL_2-wcaN | 139.7 (±14.9) | 105.0 (±3.0) |
| O-antigen and | ΔwbbK-wza | 154.4 (±15.9) | 108.6 (±2.3) |
| colanic acid KO | | | |
| O-antigen and | ΔwbbL_2-wza | 137.2 (±11.1) | 101.6 (±1.0) |
| colanic acid KO | | | |

TABLE 6

| | | Normalized | Normalized |
|---|---|---|---|
| | | LNnT CPI | Mumax |
| Mutation | Genes | (avg % ± sd) | (avg % ± sd) |
| Reference | — | 100 (±2.7) | 100 (±3.5) |
| Colanic acid KO | ΔwcaM-wza | 106.6 (±1.8) | 105.8 (±1.1) |
| O-antigen KO | ΔwbbK-wcaN | 123.6 (±1.0) | 109.0 (±1.3) |
| O-antigen KO | ΔwbbL_2-wcaN | 109.7 (±1.5) | 104.6 (±1.9) |
| O-antigen and | ΔwbbK-wza | 110.3 (±1.7) | 105.8 (±1.3) |
| colanic acid KO | | | |
| O-antigen and | ΔwbbL_2-wza | 120.5 (±4.5) | 109.4 (±2.0) |
| colanic acid KO | | | |

These strains can also be evaluated in batch or fed-batch fermentations at bioreactor scale. Such bioreactor runs can be performed as described in Example 1, with e.g., sucrose as the carbon source and lactose as the acceptor substrate. For example, such a fermentation was performed with a strain for LNnT production carrying the “Colanic acid KO” (ΔwaM-wza). During this fermentation, the LNnT titer (in g/L) and production rate (g LNnT/U/h) were on average 10% higher throughout the entire fermentation compared to an identical control bioreactor run with a reference strain lacking this ΔwaM-wza knock-out.

### Example 8: Production of Oligosaccharides in a Bacillus subtilis Host Lacking Genes for the Biosynthesis of Cell Wall Polymers Like Teichoic Acid

In another embodiment, the production of oligosaccharides, and more specifically human milk oligosaccharides such as 2′FL, 3FL, 3′-SL, 6′-SL, LNT or LNnT can be established by engineering a Bacillus subtilis host strain as described in Example 1. These strains could be modified to have deletions of particular genes in the tag gene cluster (tagOABDFGH), which includes glycosyltransferase-coding genes that are important for the biosynthesis of the cell wall polymer teichoic acid. The tagO gene, which performs the first step in teichoic acid synthesis, can be deleted with additional deletions of all or of a selection of the genes tagB, tagD, tagF, tagG or tagH. Alternatively, the tagA gene, which performs the second step in teichoic acid biosynthesis, can be deleted with additional deletions of all or of a selection of the genes tagB, tagD, tagF, tagG or tagH.

Any of these aforementioned strains are able to produce any of the listed HMO's, and in similar or potentially higher amounts than the respective reference strains lacking these cell wall structural component deletions. Additionally, the strains grow similarly well or better than their respective reference strains.

### Example 9: Production of Oligosaccharides in a Corynebacterium glutamicum Host Lacking Genes for the Biosynthesis of Cell Wall Polymers Like Corynomycolic Acids and/or Arabinogalactan

In another embodiment, the production of oligosaccharides, and more specifically human milk oligosaccharides such as 2′FL, 3FL, 3′-SL, 6′-SL, LNT or LNnT can be established by engineering a Corynebacterium glutamicum host strain as described in Example 1. These strains could be modified to have deletions of all or of a selection of the genes accD2 or accD3 in the biosynthesis pathway for corynomycolic acids. Alternatively, these strains could be modified to have deletions of all or of a selection of the genes aftA, aftB or emb, which includes glycosyltransferase-coding genes that are important in the biosynthesis of arabinogalactan, a polysaccharide structural component of the C. glutamicum cell wall. Alternatively, such strains could be modified to have deletions of multiple of the aforementioned genes that are involved in the biosynthesis of corynomycolic acids or arabinogalactan biosynthesis. The resulting strains are as such deficient in multiple of these polysaccharide structural cell wall components.

Any of these aforementioned strains are able to produce any of the listed HMO's, and in similar or potentially higher amounts than the respective reference strains lacking these cell wall structural component deletions.

### Example 10: Production of Phosphorylated and/or Activated Monosaccharides in an E. coli Host Lacking Genes for Enterobacterial Common Antigen, 0 Antigen and/or Colanic Acid Biosynthesis

E. coli strains defective in the formation of enterobacterial common antigen, 0 antigen and/or colanic acid biosynthesis, with gene deletions as listed in Example 2, can be used for the production of phosphorylated and/or activated monosaccharides. Examples of phosphorylated monosaccharides include but are not limited to glucose-1-phosphate, glucose-6-phosphate, glucose-1,6-bisophosphate, galactose-1-phosphate, fructose-6-phosphate, fructose-1,6-bisphosphate, fructose-1-phosphate, glucosamine-1-phosphate, glucosamine-6-phosphate, N-acetylglucosamine-1-phosphate, mannose-1-phosphate, mannose-6-phosphate or fucose-1-phosphate. Some but not all of these phosphorylated monosaccharides are precursors or intermediates for the production of activated monosaccharide. Examples of activated monosaccharides include but are not limited to GDP-fucose, UDP-glucose, UDP-galactose and UDP-N-acetylglucosamine. These phosphorylated monosaccharides and/or activated monosaccharides can be produced in higher amounts than naturally occurring in E. coli e.g., by introducing some of the genetic modifications as described in Example 1. An E. coli strain with active expression units of the sucrose phosphorylase and fructokinase genes (BaSP encoding the protein of SEQ ID NO:2, ZmFrk encoding the protein of SEQ ID NO:1) is able to grow on sucrose as a carbon source and can produce high(er) amounts of glucose-1P, as described in WO2012/007481. Such a strain additionally containing a knock-out of the genes pgi, pfkA and pfkB accumulate fructose-6-phosphate in the medium when grown on sucrose. Alternatively, by knocking out genes coding for (a) phosphatase(s) (agp), glucose 6-phosphate-1-dehydrogenase (zwf), phosphoglucose isomerase (pgi), glucose-1-phosphate adenylyltransferase (glgC), phosphoglucomutase (pgm) a mutant is constructed, which accumulates glucose-6-phosphate.

Alternatively, the strain according to the disclosure and further containing a sucrose phosphorylase and fructokinase with an additional overexpression of the wild type or variant protein of the L-glutamine-D-fructose-6-phosphate aminotransferase (gImS) from E. coli (encoding the protein of SEQ ID NO:6) can produce higher amounts of glucosamine-6P, glucosamine-1P and/or UDP-N-acetylglucosamine. Alternatively, by knocking out the E coli gene wcaJ coding for the undecaprenyl-phosphate glucose phosphotransferase the strain will have an increased pool of GDP-fucose. An increased pool of UDP-glucose and/or UDP-galactose could be achieved by overexpressing the E. coli enzymes glucose-1-phosphate uridyltransferase (galU) and/or UDP-galactose-4-epimerase (galE). Alternatively, by overexpressing genes coding for galactokinase (galK) and galactose-1-phosphate uridylyltransferase (for example, originating from Bifidobacterium bifidum) the formation of UDP-galactose is enhanced by additionally knocking out genes coding for (a) phosphatase(s) (agp), UDP-glucose, galactose-1P uridylyltransferase (galT), UDP-glucose-4-epimerase (galE) a mutant is constructed, which accumulates galactose-1-phosphate.

Another example of an activated monosaccharide is CMP-sialic acid, which is not naturally produced by E. coli. Production of CMP-sialic acid can e.g., be achieved by introducing genetic modifications as described in Example 1 for the 3′SL or 6′SL background strain (but without the necessity for a gene coding for a sialyltransferase enzyme).

Such strains can be used in a bio fermentation process to produce these phosphorylated monosaccharides or activated monosaccharides in which the strains are grown on e.g., one or more of the following carbon sources: sucrose, glucose, glycerol, fructose, lactose, arabinose, maltotriose, sorbitol, xylose, rhamnose and mannose.

### Example 11: Production of Monosaccharides or Disaccharides in an E. coli Host Lacking Genes for Enterobacterial Common Antigen, O Antigen and/or Colanic Acid Biosynthesis

E. coli strains defective in the formation of enterobacterial common antigen, O antigen and/or colanic acid biosynthesis, with gene deletions as listed in Example 2, can be used for the production of monosaccharides.

An example of such a monosaccharide is L-fucose. An E. coli fucose production strain can be created e.g., by starting from a strain that is able to produce 2′FL as described in Example 1 and by additionally knocking out the E. coli genes fucK and fucI (coding for an L-fucose isomerase and an L-fuculokinase) to avoid fucose degradation, and by expressing an 1,2-alpha-L-fucosidase (e.g., afcA from Bifidobacterium b fidum (GenBank accession no.: AY303700)) to degrade 2′FL into fucose and lactose. Such a strain can be used in a bio fermentation process to produce L-fucose in which the strain is grown on sucrose, glucose or glycerol and in the presence of catalytic amounts of lactose as an acceptor substrate for the alpha-1,2-fucosyltransferase.

An example of such a disaccharide is e.g., lactose (galactose-beta,1,4-glucose).

An E. coli lactose production strain can be created e.g., by introducing in wild type E. coli at least one recombinant nucleic acid sequence encoding for a protein having a beta-1,4-galactosyltransferase activity and being able to transfer galactose on a free glucose monosaccharide to intracellularly generate lactose as e.g., described in WO2015150328. As such the sucrose is taken up or internalized into the host cell via a sucrose permease. Within the bacterial host cell, sucrose is degraded by invertase to fructose and glucose. The fructose is phosphorylated by fructokinase (e.g., frk from Zymomonas mobilis (encoding the protein of SEQ ID NO:1)) to fructose-6-phosphate, which can then be further converted to UDP-galactose by the endogenous E. coli enzymes phosphohexose isomerase (pgi), phosphoglucomutase (pgm), glucose-1-phosphate uridylyltransferase (galU) and UDP-galactose-4-epimerase (galE). A beta-1,4-galactosyltransferase (e.g., igtB from Neisseria meningitidis, encoding the protein of SEQ ID NO:5) then catalyses the reaction UDP-galactose+glucose=>UDP+lactose. Preferably, the strain is further modified to not express the E coli lacZ enzyme, a beta-galatosidase, which would otherwise degrade lactose. Such a strain can be used in a bio fermentation process to produce lactose in which the strain is grown on sucrose as the sole carbon source.

### Example 12: Production of Glycolipids in an E. coli Host Lacking Genes for Enterobacterial Common Antigen, 0 Antigen and/or Colanic Acid Biosynthesis

E. coli strains defective in the formation of enterobacterial common antigen, 0 antigen and/or colanic acid biosynthesis, with gene deletions as listed in Example 2, can be used for the production of glycolipids. An example of such a glycolipid is e.g., a rhamnolipid containing one or two rhamnose residues (mono- or dirhamnolipid). The production of monorhamnolipids can be catalyzed by the enzymatic complex rhamnosyltransferase 1 (Rt1), encoded by the rhlAB operon of Pseudomonas aeruginosa, using dTDP-L-rhamnose and beta-hydroxydecanoic acid precursors. Overexpression in an E. coli strain of this rhlAB operon, as well as overexpression of the Pseudomonas aeruginosa rmlBDAC operon genes to increase dTDP-L-rhamnose availability, allows for monorhamnolipids production, mainly containing a C10-C10 fatty acid dimer moiety. This can be achieved in various media such as rich LB medium or minimal medium with glucose as carbon source.

### Example 13: Production of LNnT in a Production Host Lacking Genes for Colanic Acid and O-Antigen or Enterobacterial Common Antigen Synthesis

An E. coli strain was engineered for the production of LNnT as described in Example 1. Such a strain was further modified to additionally have a knock-out of the region in the genome encoding all or a selection of the genes rfe, wzzE, wecB, wecC, rffG, rfEH, rffC, wecE, wzxE, wecF, wzyE, rffM, wbbL_2, wbbK, wbbJ, wbbI, wbbH, glf, rfbX, rfbC, rfbA, rfbD, rfbB, wcaN, wcaM, wcaL, wcaK, wzxC, wcaJ, cpsG, cpsB, wcaI, gmm, fcl, gmd, wcaF, wcaE, wcaD, wcaC, wcaB, wcaA, wzc, wzb and wza (encoding the proteins of SEQ ID NO:15 to 58). More specifically, strains were created with a knock-out of the genes wbbL_2 to wza (encoding the proteins of SEQ ID NO:27 to 58), or of the genes wcaM to wza (encoding the proteins of SEQ ID NO:39 to 58), or of the genes wcaM to wza (encoding the proteins of SEQ ID NO:39 to 58) and rfe to rffM (encoding the proteins of SEQ ID NO:15 to 26) in a strain producing LNnT. These regions include genes that are important for the production of both colanic acid and O-antigen, or colanic acid alone, or both colanic acid and enterobacterial common antigen, respectively. The resulting mutant strains are thus deficient in one or multiple of these polysaccharide structural components.

These strains were evaluated and compared to their parent strain not lacking any of these above listed genes (“Ref”) in a growth experiment as described in Example 1. Each strain was grown in at least 4 multiple wells of a 96-well plate. The dashed horizontal line indicates the setpoint to which all datapoints were normalized.

Table 7 shows the CPI of LNnT of strains lacking important genes of both colanic acid and O-antigen, or colanic acid alone, or both colanic acid and enterobacterial common antigen, in relative % normalized to their reference strain (average value f standard deviation).

The data indicates that, compared to a reference strain, a higher CPI is obtained for LNnT production in all tested strains.

TABLE 7

| | | Normalized LNnT |
|---|---|---|
| Mutation | Genes | CPI (avg % ± sd) |
| Reference | — | 100 (±5.5) |
| Colanic acid KO | ΔwcaM-wza | 117.5 (±4.1) |
| O-antigen and | ΔwbbL_2-wza | 117.7 (±3.8) |
| colanic acid KO | | |
| Colanic acid and | ΔwcaM-wza + | 112.3 (±5.3) |
| enterobacterial common | Δrfe-rffM | |
| antigen KO | | |

### Example 14: Production of LNT by a Production Host Lacking Genes for Colanic Acid, O-Antigen and Enterobacterial Common Antigen Synthesis in a 5 L Bioreactor

An E. coli strain was engineered for the production of LNT as described in Example 1. Such a strain was further modified to additionally have a knock-out of the region in the genome encoding the genes rfe, wzzE, wecB, wecC, rffG, rfH, rffC, wecE, wzxE, wecF, wzyE, rffM, wbbL_2, wbbK, wbbJ, wbbI, wbbH, glf, rfbX, rfbC, rfbA, rfbD, rfbB, wcaN, wcaM, wcaL, wcaK, wzxC, wcaJ, cpsG, cpsB, wcaI, gmm, fcl, gmd, wcaF, wcaE, wcaD, wcaC, wcaB, wcaA, wzc, wzb and wza (encoding the proteins of SEQ ID NO:15 to 58). More specifically, a strain was created with a knock-out of the genes wbbL_2 to wza (encoding the proteins of SEQ ID NO:27 to 58) and rfe to rftM (encoding the proteins of SEQ ID NO:15 to 26) in a strain producing LNT. These regions include genes that are important for the production of both colanic acid, O-antigen and enterobacterial common antigen.

The resulting mutant strains are thus deficient in multiple of these polysaccharide structural components. This strain was evaluated and compared to the parent strain not lacking any of these above listed genes (“Ref”) in a 5 L bioreactor with 5 L working volume (BIOSTAT® B-DCU) as described in Example 1. At the end of the fermentations, the LNT and lacto-N-triose II titers varied between 75 g/L and 90 g/L (strain lacking the above listed genes) and varied between 55 g/L and 70 g/L for the parent strain. Also, filling volume of the fermentations (measured in vessels with 5.0 L working volume under the same aeration conditions) with the strain lacking the above listed genes varied between 4.6 and 4.8 L and varied between 4.8 and 5.0 L for the parent strain.

### Example 15: Materials and Methods Chlamydomonas reinhardtii

Media

C. reinhardtii cells were cultured in Tris-acetate-phosphate (TAP) medium (pH 7.0). The TAP medium uses a 1000× stock Hutner's trace element mix. Hutner's trace element mix consisted of 50 g/L Na2EDTA.H₂O (Titriplex III), 22 g/L ZnSO₄.7H₂O, 11.4 g/L H₃BO₃, 5 g/L MnCl₂.4H₂O, 5 g/L FeSO₄.7H₂O, 1.6 g/L CoCl₂.6H₂O, 1.6 g/L CuSO₄.5H₂O and 1.1 g/L (NH₄)6M0O3.

The TAP medium contained 2.42 g/L Tris (tris(hydroxymethyl)aminomethane), 25 mg/L salt stock solution, 0.108 g/L K₂HPO₄, 0.054 g/L KH₂PO₄ and 1.0 mL/L glacial acetic acid. The salt stock solution consisted of 15 g/L NH₄CL, 4 g/L MgSO₄.7H₂O and 2 g/L CaCl₂.2H₂O. Medium was sterilized by autoclaving (121° C., 21′). For stock cultures on agar slants TAP medium was used containing 1% agar (of purified high strength, 1000 g/cm²).

Strains, Plasmids and Mutations

C. reinhardtii wild-type strains 21 gr (CC-1690, wild-type, mt+), 6145C (CC-1691, wild-type, mt−), CC-125 (137c, wild-type, mt+), CC-124 (137c, wild-type, mt−) as available from Chlamydomonas Resource Center (https://www.chlamycollection.org), University of Minnesota, U.S.A.

Expression plasmids originated from pSI103, as available from Chlamydomonas Resource Center. Cloning can be performed using Gibson Assembly, Golden Gate assembly, Cliva assembly, LCR or restriction ligation. Suitable promoters for (heterologous) gene expression can be derived from e.g., Scranton et al. (Algal Res. 2016, 15: 135-142). Targeted gene modification (like gene knock-out or gene replacement) can be carried using the Crispr-Cas technology as described e.g., by Jiang et al. (Eukaryotic Cell 2014, 13(11): 1465-1469).

Transformation via electroporation was performed as described by Wang et al. (Biosci. Rep. 2019, 39: BSR2018210). Cells were grown in liquid TAP medium under constant aeration and continuous light with a light intensity of 8000 Lx until the cell density reached 1.0-2.0×10⁷ cells/mL. Then, the cells were inoculated into fresh liquid TAP medium in a concentration of 1.0×10⁶ cells/mL and grown under continuous light for 18-20 h until the cell density reached 4.0×10⁶ cells/mL. Next, cells were collected by centrifugation at 1250 g for 5 min at room temperature, washed and resuspended with pre-chilled liquid TAP medium containing 60 mM sorbitol (Sigma, U.S.A.), and iced for 10 min. Then, 250 μL of cell suspension (corresponding to 5.0×10⁷ cells) were placed into a pre-chilled 0.4 cm electroporation cuvette with 100 ng plasmid DNA (400 ng/mL). Electroporation was performed with 6 pulses of 500 V each having a pulse length of 4 ms and pulse interval time of 100 ms using a BTX ECM830 electroporation apparatus (1575 Ω, 50 μFD). After electroporation, the cuvette was immediately placed on ice for 10 min. Finally, the cell suspension was transferred into a 50 ml conical centrifuge tube containing 10 mL of fresh liquid TAP medium with 60 mM sorbitol for overnight recovery at dim light by slowly shaking. After overnight recovery, cells were recollected and plated with starch embedding method onto selective 1.5% (w/v) agar-TAP plates containing ampicillin (100 mg/L) or chloramphenicol (100 mg/L). Plates were then incubated at 23+−0.5° C. under continuous illumination with a light intensity of 8000 Lx. Cells were analyzed 5-7 days later.

For enhanced production of endogenous and/or exogenous oligomannoside N-glycosylated glycoproteins, C. reinhardtii cells were modified with a transcriptional unit comprising the At1g3000 gene from Arabidopsis thaliana encoding an a-1,2-mannosidase that is involved in the trimming of N-linked glycans in the Golgi apparatus. In a next step for production of xylosylated oligomannoside N-glycosylated glycoproteins, mutant C. reinhardtii cells were transformed with an expression plasmid comprising a transcriptional unit for the At5g55500 gene from A. thaliana encoding a beta-1,2-xylosyltransferase that transfers xylose to the mannose subunits present in the N-glycan(s) of N-glycosylated proteins.

For enhanced production of endogenous and/or exogenous glycolipids C. reinhardtii cells were transformed with an expression plasmid comprising an overexpression unit for GTR14, encoding the GPI mannosyltransferase I, which is involved in the transfer of the first alpha-1,4-mannose to GlcN-acyl-PI during GPI precursor assembly.

Heterologous and Homologous Expression

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

Cultivation Conditions

Cells of C. reinhardtii were cultured in selective TAP-agar plates at 23+−0.5° C. under 14/10 h light/dark cycles with a light intensity of 8000 Lx. Cells were analyzed after 5 to 7 days of cultivation.

For high-density cultures, cells could be cultivated in closed systems like e.g., vertical or horizontal tube photobioreactors, stirred tank photobioreactors or flat panel photobioreactors as described by Chen et al. (Bioresour. Technol. 2011, 102: 71-81) and Johnson et al. (Biotechnol. Prog. 2018, 34: 811-827).

### Example 16: Production of Endogenous and/or Exogenous N-Glycosylated Proteins in a C. reinhardtii Host Lacking a Gene for Beta-1,3-Glucan Biosynthesis and/or Deficient in Hydroxyproline-Rich Glycoproteins

C. reinhardtii mutant strains for enhanced production of endogenous and/or exogenous oligomannoside N-glycoproteins and xylosylated oligomannoside N-glycoproteins are engineered as described in Example 15. Such strains are further modified via Crispr-Cas technology to additionally have a deletion in or a knock-out in any one or more of the GTR13 gene encoding 1,3-beta-D-glucan synthase, or the SAG1, SAD1, GP1, GP2 or VSP3 genes encoding hydroxyproline-rich glycoproteins (HRGPs). The resulting strains are thus deficient in the synthesis of beta-1,3-glucan and/or specific HRGPs as important cell wall components of C. reinhardtii.

### Example 17: Production of Rhamnolipids in a C. reinhardtii Host Lacking a Gene for Beta-Glucan Biosynthesis

C. reinhardtii mutant strains were engineered for production of a rhamnolipid, e.g., a rhamnolipid containing one or two rhamnose residues (mono- or dirhamnolipid). Therefore, C. reinhardtii cells were transformed with an expression plasmid comprising the rhlAB operon of Pseudomonas aeruginosa, encoding for the rhamnosyltransferase 1 (Rt1) complex, and the rmlBDAC operon genes of Pseudomonas aeruginosa, to increase dTDP-L-rhamnose availability, allowing for monorhamnolipids production, mainly containing a C10-C10 fatty acid dimer moiety. The novel strains were further engineered via Crispr-Cas technology to additionally have a deletion in or a knock-out in the GTR13 gene encoding 1,3-beta-D-glucan synthase. The resulting strains are thus deficient in the synthesis of beta-1,3-glucan as important cell wall component of C. reinhardtii.

1.-46. (canceled)

47. A microorganism genetically modified for production of at least one glycosylated product,
wherein the microorganism is a bacterium or yeast, and
wherein the microorganism has a cell wall biosynthesis that is reduced by a deletion, reduced, or abolished expression of at least one enzyme within the cell wall biosynthesis pathway, which cell wall biosynthesis pathway is at least one pathway selected from the group consisting of:
cell wall carbohydrate antigen biosynthesis,
capsular polysaccharide biosynthesis,
cell wall protein mannosylation biosynthesis, beta-1,3-glucan biosynthesis, beta-1,6-glucan biosynthesis and/or chitin biosynthesis when the microorganism is a yeast,
mycolic acid and/or arabinogalactan biosynthesis when the microorganism is a Corynebacterium, Nocardia, or Mycobacterium, and
teichoic acid biosynthesis when the microorganism is a Gram-positive bacterium.

48. The microorganism of claim 47, wherein the reduced cell wall biosynthesis pathway is combined with the introduction of one or more pathways for the synthesis of one or more nucleotide-activated sugars.

49. The microorganism of claim 47, wherein the microorganism is further modified to express one or more glycosyltransferases for production of the glycosylated product.

50. The microorganism of claim 47, wherein the glycosylated product is an oligosaccharide, a glycosylated aglycon, a glycolipid, or a glycoprotein.

51. The microorganism of claim 47, wherein the enzyme within the cell wall biosynthesis pathway is a glycosyltransferase.

52. The microorganism of claim 47, wherein the microorganism is a bacterium selected from the group consisting of Escherichia, Bacillus, Lactobacillus, Lactococcus, and Corynebacterium.

53. The microorganism of claim 47, wherein the microorganism is a yeast selected from the group consisting of Pichia, Hansenula, Komagataella, and Saccharomyces.

54. The microorganism of claim 47, wherein the microorganism is a bacterium having a further cell wall biosynthesis pathway that is reduced by a deletion, reduced, or abolished expression of at least one enzyme within the further cell wall biosynthesis pathway selected from the group consisting of colanic acid biosynthesis, exopolysaccharide biosynthesis lipopolysaccharide biosynthesis, and a combination of any thereof.

55. The microorganism of claim 47, wherein the microorganism is a Gram-negative bacterium having a reduced cell wall biosynthesis by a reduced O-antigen biosynthesis wherein the reduction in the O-antigen biosynthesis is provided by a deletion, reduced or abolished expression of at least one of the genes present in the O-antigen biosynthesis gene cluster comprising rhamnosyltransferase, putative glycosyltransferase, putative lipopolysaccharide biosynthesis O-acetyl transferase, β-1,6-galactofuranosyltransferase, putative O-antigen polymerase, UDP-galactopyranose mutase, polyisoprenol-linked O-antigen repeat unit flippase, dTDP-4-dehydrorhamnose 3,5-epimerase, dTDP-glucose pyrophosphorylase, dTDP-4-dehydrorhamnose reductase, dTDP-glucose 4,6-dehydratase 1, or UTP:glucose-1-phosphate uridylyltransferase.

56. The microorganism of claim 55, wherein the reduction in the O-antigen biosynthesis is provided by a deletion, reduced or abolished expression of at least one of i) WbbL, WbbK, WbbJ, WbbI, WbbH, glf, rfbX, rfbC, rfbA, rfbD, rfbB, wcaN, optionally as given by SEQ ID NOs:27 to 38, respectively, or ii) a polypeptide sequence having 80% or more sequence identity to the full-length sequence of any one of the SEQ ID NOs:27 to 38 and having rhamnosyltransferase activity, glycosyltransferase activity, lipopolysaccharide biosynthesis 0-acetyl transferase activity, β-1,6-galactofuranosyltransferase activity, O-antigen polymerase activity, UDP-galactopyranose mutase activity, polyisoprenol-linked O-antigen repeat unit flippase activity, dTDP-4-dehydrorhamnose 3,5-epimerase activity, dTDP-glucose pyrophosphorylase activity, dTDP-4-dehydrorhamnose reductase activity, dTDP-glucose 4,6-dehydratase 1 activity or UTP:glucose-1-phosphate uridylyltransferase activity, respectively.

57. The microorganism of claim 47, wherein the microorganism is a Gram-negative bacterium having a reduced cell wall biosynthesis by a reduced common-antigen biosynthesis, wherein the reduction in the common-antigen biosynthesis is provided by a deletion, reduced or abolished expression of at least one of the genes present in the common-antigen biosynthesis gene cluster comprising UDP-N-acetylglucosamine-undecaprenyl-phosphate N-acetylglucosaminephosphotransferase, enterobacterial common antigen polysaccharide co-polymerase, UDP-N-acetylglucosamine 2-epimerase, UDP-N-acetyl-D-mannosamine dehydrogenase, dTDP-glucose 4,6-dehydratase 2, dTDP-glucose pyrophosphorylase, dTDP-4-amino-4,6-dideoxy-D-galactose acyltransferase, dTDP-4-dehydro-6-deoxy-D-glucose transaminase, lipid III flippase, TDP-N-acetylfucosamine:lipid II N-acetylfucosaminyltransferase, putative enterobacterial common antigen polymerase, or UDP-N-acetyl-D-mannosaminuronic acid transferase.

58. The microorganism of claim 57, wherein the reduction in the common-antigen biosynthesis is provided by a deletion, reduced or abolished expression of at least one of i) rfe, wzzE, wecB, wecC, rffG, rffH, rffC, wecE, wzxE, wecF, wzyE, rfM, optionally as given by SEQ ID NOs:15 to 26, respectively, or ii) a polypeptide sequence having 80% or more sequence identity to the full-length sequence of any one of the SEQ ID NOs:15 to 26 and having UDP-N-acetylglucosamine-undecaprenyl-phosphate N-acetylglucosaminephosphotransferase activity, enterobacterial common antigen polysaccharide co-polymerase activity, UDP-N-acetylglucosamine 2-epimerase activity, UDP-N-acetyl-D-mannosamine dehydrogenase activity, dTDP-glucose 4,6-dehydratase 2 activity, dTDP-glucose pyrophosphorylase activity, dTDP-4-amino-4,6-dideoxy-D-galactose acyltransferase activity, dTDP-4-dehydro-6-deoxy-D-glucose transaminase activity, lipid III flippase activity, TDP-N-acetylfucosamine:lipid II N-acetylfucosaminyltransferase activity, enterobacterial common antigen polymerase activity or UDP-N-acetyl-D-mannosaminuronic acid transferase activity, respectively.

59. The microorganism of claim 54, wherein the microorganism is a bacterium having a further reduced cell wall biosynthesis by a reduced colanic acid biosynthesis, wherein the reduction in the colanic acid biosynthesis is provided by a deletion, reduced or abolished expression of at least one of the genes present in the colanic acid biosynthesis gene cluster comprising putative colanic acid biosynthesis protein, putative colanic biosynthesis glycosyl transferase, putative colanic acid biosynthesis pyruvyl transferase, M-antigen undecaprenyl diphosphate flippase, UDP-glucose:undecaprenyl-phosphate glucose-1-phosphate transferase, phosphomannomutase, mannose-1-phosphate guanylyltransferase, colanic acid biosynthesis fucosyltransferase, GDP-mannose mannosyl hydrolase, GDP-L-fucose synthase, GDP-mannose 4,6-dehydratase, colanic acid biosynthesis acetyltransferase, colanic acid biosynthesis fucosyltransferase, putative colanic acid polymerase, colanic acid biosynthesis galactosyltransferase, colanic acid biosynthesis acetyltransferase, colanic acid biosynthesis glucuronosyltransferase, protein-tyrosine kinase, protein-tyrosine phosphatase, or outer membrane polysaccharide export protein.

60. The microorganism of claim 59, wherein the reduction in the colanic acid biosynthesis is provided by a deletion, reduced or abolished expression of at least one of i) WcaM, WcaL, WcaK, WzxC, wcaJ, cpsG, cpsB, WcaI, gmm, fcl, gmd, WcaF, WcaE, WcaD, WcaC, WcaB, WcaA, Wzc, wzb, Wza, optionally as given by SEQ ID NOs:39 to 58, respectively, or ii) a polypeptide sequence having 80% or more sequence identity to the full-length sequence of any one of the SEQ ID NOs:39 to 58 and having colanic acid biosynthesis protein activity, colanic biosynthesis glycosyl transferase activity, colanic acid biosynthesis pyruvyl transferase activity, M-antigen undecaprenyl diphosphate flippase activity, UDP-glucose:undecaprenyl-phosphate glucose-1-phosphate transferase activity, phosphomannomutase activity, mannose-1-phosphate guanylyltransferase activity, colanic acid biosynthesis fucosyltransferase activity, GDP-mannose mannosyl hydrolase activity, GDP-L-fucose synthase activity, GDP-mannose 4,6-dehydratase activity, colanic acid biosynthesis acetyltransferase activity, colanic acid biosynthesis fucosyltransferase activity, colanic acid polymerase activity, colanic acid biosynthesis galactosyltransferase activity, colanic acid biosynthesis acetyltransferase activity, colanic acid biosynthesis glucuronosyltransferase activity, protein-tyrosine kinase activity, protein-tyrosine phosphatase activity or outer membrane polysaccharide export protein activity, respectively.

61. The microorganism of claim 47, wherein the microorganism is a yeast having a reduced cell wall biosynthesis by a reduced cell wall protein mannosylation biosynthesis, wherein the reduction of the cell wall protein mannosylation biosynthesis is provided by a deletion, reduced or abolished expression of at least one of Protein-O-mannosyltransferase encoding gene.

62. The microorganism of claim 47, wherein the microorganism is a Corynebacterium, Nocardia, or Mycobacterium having a reduced cell wall biosynthesis by a reduced mycolic acid and/or arabinogalactan biosynthesis wherein the reduced mycolic acid and/or arabinogalactan biosynthesis is provided by a reduced expression of at least one of mycolic acid and/or arabinogalactan biosynthesis genes.

63. The microorganism of claim 47, wherein the microorganism is a Gram-positive bacterium having a reduced cell wall biosynthesis by a reduced teichoic acid biosynthesis wherein the reduced teichoic acid biosynthesis is provided by a reduced expression of at least one of teichoic acid biosynthesis genes.

64. The microorganism of claim 47, wherein the glycosylated product is an oligosaccharide with a degree of polymerization greater than 3.

65. The microorganism of claim 47, wherein the microorganism is isolated.

66. A method to reduce the viscosity, foaming, and/or airlift of a fermentation process with a microorganism, wherein the cell wall biosynthesis of the microorganism is reduced by deletion, reduced or abolished expression of at least one enzyme within the cell wall biosynthesis pathway,
wherein the microorganism is a bacterium or yeast, and
wherein the cell wall biosynthesis pathway is at least one pathway selected from the group consisting of:
cell wall carbohydrate antigen biosynthesis,
capsular polysaccharide biosynthesis,
cell wall protein mannosylation biosynthesis, beta-1,3-glucan biosynthesis, beta-1,6-glucan biosynthesis and/or chitin biosynthesis when the microorganism is a yeast, mycolic acid and/or arabinogalactan biosynthesis when the microorganism is a Corynebacterium, Nocardia or Mycobacterium, and
teichoic acid biosynthesis when the microorganism is a Gram-positive bacterium.

67. The method according to claim 66, wherein the microorganism is further modified to produce at least one glycosylated product.

68. A method for producing glycosylated product by a genetically modified cell, the method comprising:
culturing a cell in a medium under conditions permissive for producing glycosylated product, wherein the cell is genetically modified for producing glycosylated product, the cell comprising at least one polynucleotide encoding an enzyme for glycosylated product synthesis, wherein the cell is further genetically modified for reduced cell wall biosynthesis by deletion, reduced or abolished expression of at least one enzyme within the cell wall biosynthesis pathway, wherein the cell wall biosynthesis pathway is at least one pathway chosen from cell wall carbohydrate antigen biosynthesis, capsular polysaccharide biosynthesis, cell wall protein mannosylation biosynthesis, beta-1,3-glucan biosynthesis, beta-1,6-glucan biosynthesis, chitin biosynthesis, mycolic acid biosynthesis, arabinogalactan biosynthesis and teichoic acid biosynthesis, and
optionally separating glycosylated product from the culture.

69. The method according to claim 68, wherein the enzyme for glycosylated product synthesis comprises enzymes involved in nucleotide-activated sugar synthesis and glycosyltransferases.

70. The method according to claim 68, wherein the genetically modified cell is a bacterium or yeast.

71. The method according to claim 68, wherein the genetically modified cell is a bacterium, which is selected from the group consisting of Enterobacteriaceae and Escherichia.

72. The method according to claim 68, wherein the genetically modified cell is a yeast selected from the group consisting of Pichia, Hansenula, Komagataella, and Saccharomyces.

73. A method for producing glycosylated product by a genetically modified Gram-negative bacterial cell, the method comprising:
culturing a cell in a medium under conditions permissive for producing glycosylated product, wherein the cell is a Gram-negative bacterial cell genetically modified for producing glycosylated product, the cell comprising at least one polynucleotide encoding an enzyme for glycosylated product synthesis, the cell further genetically modified for reduced cell wall biosynthesis by deletion, reduced or abolished expression of at least one enzyme within the cell wall biosynthesis pathway, the cell wall biosynthesis being cell wall carbohydrate antigen biosynthesis, and
optionally separating glycosylated product from the culture.

74. The method according to claim 73, wherein the enzyme for glycosylated product synthesis comprises enzymes involved in nucleotide-activated sugar synthesis and glycosyltransferases.

75. The method according to claim 73, wherein the Gram-negative bacterial cell has a further cell wall biosynthesis pathway that is reduced by a deletion, reduced or abolished expression of at least one enzyme within the further cell wall biosynthesis pathway chosen from colanic acid biosynthesis, exopolysaccharide biosynthesis and/or lipopolysaccharide biosynthesis.

76. A method for producing glycosylated product by a genetically modified yeast cell, the method comprising:
culturing a cell in a medium under conditions permissive for producing glycosylated product, wherein the cell is a yeast cell genetically modified for producing glycosylated product, the cell comprising at least one polynucleotide encoding an enzyme for glycosylated product synthesis, the cell further genetically modified for reduced cell wall biosynthesis by deletion, reduced or abolished expression of at least one enzyme within the cell wall biosynthesis pathway, the cell wall biosynthesis being i) cell wall protein mannosylation biosynthesis, ii) beta-1,3-glucan biosynthesis, iii) beta-1,6-glucan biosynthesis, and/or iv) chitin biosynthesis, and
optionally separating glycosylated product from the culture.

77. The method according to claim 76, wherein the enzyme for glycosylated product synthesis comprises enzymes involved in nucleotide-activated sugar synthesis and glycosyltransferases.

78. A method for producing glycosylated product by a genetically modified Corynebacterium, Nocardia, or Mycobacterium cell, the method comprising:
culturing a cell in a medium under conditions permissive for producing glycosylated product, wherein the cell is a Corynebacterium, Nocardia, or Mycobacterium cell genetically modified for producing glycosylated product, the cell comprising at least one polynucleotide encoding an enzyme for glycosylated product synthesis, the cell further genetically modified for reduced cell wall biosynthesis by deletion, reduced or abolished expression of at least one enzyme within the cell wall biosynthesis pathway, the cell wall biosynthesis being i) mycolic acid biosynthesis, and/or ii) arabinogalactan biosynthesis, and
optionally separating glycosylated product from the culture.

79. The method according to claim 78, wherein the enzyme for glycosylated product synthesis comprises enzymes involved in nucleotide-activated sugar synthesis and glycosyltransferases.

80. A method for producing glycosylated product by a genetically modified Bacillus cell, the method comprising:
culturing a cell in a medium under conditions permissive for producing glycosylated product, wherein the cell is a Bacillus cell genetically modified for producing glycosylated product, the cell comprising at least one polynucleotide encoding an enzyme for glycosylated product synthesis, the cell further genetically modified for reduced cell wall biosynthesis by deletion, reduced or abolished expression of at least one enzyme within the cell wall biosynthesis pathway, the cell wall biosynthesis being teichoic acid biosynthesis, and
optionally separating glycosylated product from the culture.

81. The method according to claim 80, wherein the enzyme for glycosylated product synthesis comprises enzymes involved in nucleotide-activated sugar synthesis and glycosyltransferases.

82. A method for producing glycosylated product, the method comprising:
culturing a cell in a medium under conditions permissive for producing the glycosylated product, wherein the cell is a cell of the microorganism of claim 47, and
optionally separating the glycosylated product from the culture.

83. The method according to claim 66, wherein the cell wall biosynthesis is reduced by deletion, reduced or abolished expression of at least one glycosyltransferase within the cell wall biosynthesis pathway.

84. The method according to claim 66, wherein the glycosylated product is chosen from saccharide, a glycosylated aglycon, a glycolipid, or a glycoprotein.

85. The method according to claim 66, wherein the glycosylated product is an oligosaccharide or a mammalian milk oligosaccharide.

86. The method according to claim 66, wherein the glycosylated product is an oligosaccharide with a degree of polymerization higher than 3.

87. The method according to claim 86, wherein the oligosaccharide is a mammalian milk oligosaccharide.

88. The method according to claim 68, wherein the cell wall biosynthesis is reduced by deletion, reduced or abolished expression of at least one glycosyltransferase within the cell wall biosynthesis pathway.

89. The method according to claim 68, wherein the glycosylated product is chosen from saccharide, a glycosylated aglycon, a glycolipid, or a glycoprotein.

90. The method according to claim 68, wherein the glycosylated product is an oligosaccharide or a mammalian milk oligosaccharide.

91. The method according to claim 68, wherein the glycosylated product is an oligosaccharide with a degree of polymerization higher than 3.

92. The method according to claim 91, wherein the oligosaccharide is a mammalian milk oligosaccharide.

93. A method of using the microorganism of claim 47, the method comprising:
using the microorganism to produce an oligosaccharide or a mammalian milk oligosaccharide.

94. The method according to claim 73, wherein the cell is an Escherichia coli cell.

95. A method of producing glycosylated product by a genetically modified cell in a bioreactor, the method comprising:
culturing a cell in a medium under conditions permissive for producing glycosylated product,
wherein the cell is genetically modified for producing glycosylated product, the cell comprising at least one polynucleotide encoding an enzyme for glycosylated product synthesis, and
wherein the vessel filling of the bioreactor is greater than or equal to 50%.

96. The method according to claim 95, wherein the enzyme for glycosylated product synthesis comprises enzymes involved in nucleotide-activated sugar synthesis and glycosyltransferases.

97. The method according to claim 95, wherein the cell is a bacterium or yeast, having a cell wall biosynthesis reduced by a deletion, reduced, or abolished expression of at least one enzyme within the cell wall biosynthesis pathway, which cell wall biosynthesis pathway is selected from the group consisting of cell wall carbohydrate antigen biosynthesis, capsular polysaccharide biosynthesis, cell wall protein mannosylation biosynthesis, beta-1,3-glucan biosynthesis, beta-1,6-glucan biosynthesis and/or chitin biosynthesis when the cell is a yeast, mycolic acid and/or arabinogalactan biosynthesis when the cell is a Corynebacterium, Nocardia, or Mycobacterium, and teichoic acid biosynthesis when the cell is a Gram-positive bacterium.

98. The method according to claim 95, wherein the glycosylated product is an oligosaccharide, a mammalian milk oligosaccharide, fucosylated oligosaccharide, neutral oligosaccharide or sialylated oligosaccharide, 2′-fucosyllactose, 3-fucosyllactose, difucosyllactose, Lacto-N-tetraose, Lacto-N-neotetraose, 3′-sialyllactose, 6′-sialyllactose, lacto-N-fucopentaose II, lacto-N-fucopentaose I, lacto-N-fucopentaose III, lacto-N-fucopentaose V, lacto-N-fucopentaose VI, sialyllacto-N-tetraose d (LSTd), sialyllacto-N-tetraose c (LSTc), sialyllacto-N-tetraose b (LSTb), or sialyllacto-N-tetraose a (LSTa).


SEQUENCE LISTING (58 sequences)

SEQ ID NO: 1 - protein, 301 aa, Zymomonas mobilis
MKNDKKIYGCIEGGGTKFMLALIDSDRKMLAVERVPTTTPEETLGKSVEFFKKALPQYADSFASFGIASFGPLCLDRKSPKWGYITNTPKPFWPNTDVVTPFKEAFGCPVEIDTDVNGAALAENFWGASKGTHTSVYVTVGTGFGGGVLIDGKPIHGLAHPEMGHGIPIRHPDDRDFEGCCPYHGGCYEGLASGTAIRKRWGKALNEMEPAEFEKAREIIAFYLAHFNVTLQAFISPERIVFGGGVMHVDGMLASVRRQTAEIANSYFEGADFEKIIVLPGLGDQAGMMGAFALALAAENK

SEQ ID NO: 2 - protein, 504 aa, Bifidobacterium adolescentis [sequence omitted]

SEQ ID NO: 3 - protein, 348 aa, Neisseria meningitidis
MPSEAFRRHRAYRENKLQPLVSVLICAYNVEKYFAQSLAAVVNQTWCNLDILIVDDGSTDGTLAIAQRFQEQDGRIKILAQAQNSGLIPSLNIGLDELAKSGMGEYIARTDADDIAAPDWIEKIVGEMEKDRSIIAMGAWLEVLSEEKDGNRLARHHEHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGVWLDFAANGRMRRLFTLRQYFGILHRLLKNR

SEQ ID NO: 4 - protein, 265 aa, Escherichia coli
MIIDEAESAESTHPVVSVILPVNKKNPFLDEAINSILSQTFSSFEIIIVANCCTDDFYNELKHKVNDKIKLIRTNIAYLPYSLNKAIDLSNGEFIARMDSDDISHPDRFTKQVDFLKNNPYVDVVGTNAIFIDDKGREINKTKLPEENLDIVKNLPYKCCIVHPSVMFRKKVIASIGGYMFSNYSEDYELWNRLSLAKIKFQNLPEYLFYYRLHEGQSTAKKNLYMVMVNDLVIKMKCFFLTGNINYLFGGIRTIASFIYCKYIK

SEQ ID NO: 5 - protein, 275 aa, Neisseria meningitidis
MQNHVISLASAAERRAHIADTFGRHGIPFQFFDALMPSERLEQAMAELVPGLSAHPYLSGVEKACFMSHAVLWKQALDEGLPYITVFEDDVLLGEGAEKFLAEDAWLQERFDPDTAFIVRLETMFMHVLTSPSGVADYCGRAFPLLESEHWGTAGYIISRKAMRFFLDRFAALPPEGLHPVDLMMFSDFFDREGMPVCQLNPALCAQELHYAKFHDQNSALGSLIEHDRLLNRKQQRRDSPANTFKHRLIRALTKISREREKRRQRREQFIVPFQ

SEQ ID NO: 6 - protein, 609 aa, Escherichia coli
MCGIVGAIAQRDVAEILLEGLRRLEYRGYDSAGLAVVDTEGHMTRLRRLGKVQMLAQAAEEHPLHGGTGIAHTRWATHGEPSEVNAHPHVSEHIVVVHNGIIENHEPLREELKARGYTFVSETDTEVIAHLVNWELKQGGTLREAVLRAIPQLRGAYGTVIMDSRHPDTLLAARSGSPLVIGLGMGENFIASDQLALLPVTRRFIFLEEGDIAEITRRSVNIFDKTGAEVKRQDIESNLQYDAGDKGIYCHYMQKEIYEQPNAIKNTLTGRISHGQVDLSELGPNADELLSKVEHIQILACGTSYNSGMVSRYWFESLAGIPCDVEIASEFRYRKSAVRRNSLMITLSQSGETADTLAGLRLSKELGYLGSLAICNVPGSSLVRESDLALMTNAGTEIGVASTKAFTTQLTVLLMLVAKLSRLKGLDASIEHDIVHGLQALPSRIEQMLSQDKRIEALAEDFSDKHHALFLSRGDQYPIALEGALKLKEISYIHAEAYAAGELKHGPLALIDADMPVIVVAPNNELLEKLKSNIEEVRARGGQLYVFADQDAGFVSSDNMHIIEMPHVEEVIAPIFYTVPLQLLAYHVALIKGTDVDQPRNLAKSVTVE

SEQ ID NO: 7 - protein, 159 aa, Saccharomyces cerevisiae
MSLPDGFYIRRMEEGDLEQVTETLKVLTTVGTITPESFSKLIKYWNEATVWNDNEDKKIMQYNPMVIVDKRTETVAATGNIIIERKIIHELGLCGHIEDIAVNSKYQGQGLGKLLIDQLVTIGFDYGCYKIILDCDEKNVKFYEKCGFSNAGVEMQIRK

SEQ ID NO: 8 - protein, 421 aa, Bacteroides ovatus
MDSKNNIGHSADISLTAELPIPIYNGNTIMDFKKLASLYKDELLDNVLPFWLEHSQDHEYGGYFTCLDREGKVFDTDKFIWLQSREVWMFSMLYNKVEKRQEWLDCAIQGGEFLKKYGHDGNYNWYFSLDRSGRPLVEPYNIFSYTFATMAFGQLSLTTGNQEYADIAKKTFDIILSKVDNPKGRWNKLHPGTRNLKNFALPMILCNLALEIEHLLDETYLRETMDTCIHEVMEVFYRPELGGIIVENVDIDGNLVDCFEGRQVTPGHAIEAMWFIMDLGKRLNRPELIEKAKETTLTMLNYGWDKQYGGIYYFMDRNGCPPQQLEWDQKLWWVHIETLISLLKGYQLTGDKKCLEWFEKVHDYTWEHFKDKEYPEWYGYLNRRGEVLLPLKGGKWKGCFHVPRGLYQCWKTLEEIKNIVS

SEQ ID NO: 9 - protein, 346 aa, Campylobacter jejuni
MKEIKIQNIIISEEKAPLVVPEIGINHNGSLELAKIMVDAAFSAGAKIIKHQTHIVEDEMSKAAKKVIPGNAKISIYEIMQKCALDYKDELALKEYTEKLGLVYLSTPFSRAGANRLEDMGVSAFKIGSGECNNYPLIKHIAAFKKPMIVSTGMNSIESIKPTVKILLDNEIPFVLMHTTNLYPTPHNLVRLNAMLELKKEFSCMVGLSDHTTDNLACLGAVVLGACVLERHFTDSMHRSGPDIVCSMDTKALKELIIQSEQMAIIRGNNESKKAAKQEQVTIDFAFASVVSIKDIKKGEVLSMDNIWVKRPGLGGISAAEFENILGKKALRDIENDAQLSYEDFA

SEQ ID NO: 10 - protein, 221 aa, Campylobacter jejuni
MSLAIIPARGGSKGIKNKNLVLLNNKPLIYYTIKAALNAKSISKVVVSSDSDEILNYAKSQNVDILKRPISLAQDDTTSDKVLLHALKFYKDYEDVVFLQPTSPLRTNIHINEAFNLYKNSNANALISVSECDNKILKAFVCNDCGDLAGICNDEYPFMPRQKLPKTYMSNGAIYILKIKEFLNNPSFLQSKTKHFLMDESSSLDIDCLEDLKKVEQIWKK

SEQ ID NO: 11 - protein, 268 aa, Pasteurella multocida
MDKFAEHEIPKAVIVAGNGESLSQIDYRLLPKNYDVFRCNQFYFEERYFLGNKIKAVFFTPGVFLEQYYTLYHLKRNNEYFVDNVILSSFNHPTVDLEKSQKIQALFIDVINGYEKYLSKLTAFDVYLRYKELYENQRITSGVYMCAVAIAMGYTDIYLTGIDFYQASEENYAFDNKKPNIIRLLPDFRKEKTLFSYHSKDIDLEALSFLQQHYHVNFYSISPMSPLSKHFPIPTVEDDCETTFVAPLKENYINDILLPPHFVYEKLG

SEQ ID NO: 12 - protein, 391 aa, Photobacterium damselae
MTVVAPTLEVYIDHASLPSLQQLIHIIQAKDEYPSNQRFVSWKRVTVDADNANKLNIHTYPLKGNNTSPEMVAAIDEYAQSKNRLNIEFYTNTAHVFNNLPPIIQPLYNNEKVKISHISLYDDGSSEYVSLYQWKDTPNKIETLEGEVSLLANYLAGTSPDAPKGMGNRYNWHKLYDTDYYFLREDYLDVEANLHDLRDYLGSSAKQMPWDEFAKLSDSQQTLFLDIVGFDKEQLQQQYSQSPLPNFIFTGTTTWAGGETKEYYAQQQVNVINNAINETSPYYLGKDYDLFFKGHPAGGVINDIILGSFPDMINIPAKISFEVLMMTDMLPDTVAGIASSLYFTIPADKVNFIVFTSSDTITDREEALKSPLVQVMLTLGIVKEKDVLFWA

SEQ ID NO: 13 - protein, 299 aa, Helicobacter pylori
MAFKVVQICGGLGNQMFQYAFAKSLQKHSNTPVLLDITSFDWSNRKMQLELFPIDLPYASEKEIAIAKMQHLPKLVRNVLKCMGFDRVSQEIVFEYEPKLLKTSRLTYFYGYFQDPRYFDAISPLIKQTFTLPPPPENGNNKKKEEEYHRKLALILAAKNSVFVHIRRGDYVGIGCQLGIDYQKKALEYMAKRVPNMELFVFCEDLEFTQNLDLGYPFMDMTTRDKEEEAYWDMLLMQSCKHGIIANSTYSWWAAYLINNPEKIIIGPKHWLFGHENILCKEWVKIESHFEVKSQKYNA

SEQ ID NO: 14 - protein, 329 aa, Basilea psittacipulmonis
MSVLKKLVRTLKKKKDIPSENQEDIKPQEFGHIKHYHFWPLSNETFFNQFAQEKNLDLSQTALISCFGELSAIPKIPERYKVFFTGENIYHPDRISYSDPELYRMVDLYLGFEYRTEPKYLRFPLWVWYLCGLTKKPHFSHESIAEFIRKMNQPEFRLQSSRNRFCSHISSHDTNGIRKRMIDLILPIASVDCAGKFMNNTDELKAKFNDDKIDYLKQYRFNLCPENSESVGYITEKIFESIMAGCIPIYWGGVKQLFVEPDILNPEAFIYYEKGKEEQLAKQVEELWISPKRYEEFAAIAPFKEDAAEVIYTWIEELEKRLRAFEPKA

SEQ ID NO: 15 - protein, 367 aa, Excherichia coli
MNLLTVSTDLISIFLFTTLFLFFARKVAKKVGLVDKPNFRKRHQGLIPLVGGISVYAGICFTFGIVDYYIPHASLYLACAGVLVFIGALDDRFDISVKIRATIQAAVGIVMMVFGKLYLSSLGYIFGSWEMVLGPFGYFLTLFAVWAAINAFNMVDGIDGLLGGLSCVSFAAIGMILWFDGQTSLAIWCFAMIAAILPYIMLNLGILGRRYKVFMGDAGSTLIGFTVIWILLETTQGKTHPISPVTALWIIAIPLMDMVAIMYRRLRKGMSPFSPDRQHIHHLIMRAGFTSRQAFVLITLAAALLASIGVLAEYSHFVPEWVMLVLFLLAFFLYGYCIKRAWKVARFIKRVKRRLRRNRGGSPNLTK

SEQ ID NO: 16 - protein, 348 aa, Excherichia coli
MTQPMPGKPAEDAENELDIRGLFRTLWAGKLWIIGMGLAFALIALAYTFFARQEWSSTAITDRPTVNMLGGYYSQQQFLRNLDVRSNMASADQPSVMDEAYKEFVMQLASWDTRREFWLQTDYYKQRMVGNSKADAALLDEMINNIQFIPGDFTRAVNDSVKLIAETAPDANNLLRQYVAFASQRAASHLNDELKGAWAARTIQMKAQVKRQEEVAKAIYDRRMNSIEQALKIAEQHNISRSATDVPAEELPDSEMFLLGRPMLQARLENLQAVGPAFDLDYDQNRAMLNTLNVGPTLDPRFQTYRYLRTPEEPVKRDSPRRAFLMIMWGIVGGLIGAGVALTRRCSK

SEQ ID NO: 17 - protein, 376 aa, Excherichia coli
MKVLTVFGTRPEAIKMAPLVHALAKDPFFEAKVCVTAQHREMLDQVLKLFSIVPDYDLNIMQPGQGLTEITCRILEGLKPILAEFKPDVVLVHGDTTTTLATSLAAFYQRIPVGHVEAGLRTGDLYSPWPEEANRTLTGHLAMYHFSPTETSRQNLLRENVADSRIFITGNTVIDALLWVRDQVMSSDKLRSELAANYPFIDPDKKMILVTGHRRESFGRGFEEICHALADIATTHQDIQIVYPVHLNPNVREPVNRILGHVKNVILIDPQEYLPFVWLMNHAWLILTDSGGIQEEAPSLGKPVLVMRDTTERPEAVTAGTVRLVGTDKQRIVEEVTRLLKDENEYQAMSRAHNPYGDGQACSRILEALKNNRISL

SEQ ID NO: 18 - protein, 420 aa, Excherichia coli
MSFATISVIGLGYIGLPTAAAFASRQKQVIGVDINQHAVDTINRGEIHIVEPDLASVVKTAVEGGFLRASTTPVEADAWLIAVPTPFKGDHEPDMTYVESAARSIAPVLKKGALVILESTSPVGSTEKMAEWLAEMRPDLTFPQQVGEQADVNIAYCPERVLPGQVMVELIKNDRVIGGMTPVCSARASELYKIFLEGECVVTNSRTAEMCKLTENSFRDVNIAFANELSLICADQGINVWELIRLANRHPRVNILQPGPGVGGHCIAVDPWFIVAQNPQQARLIRTAREVNDHKPFWVIDQVKAAVADCLAATDKRASELKIACFGLAFKPNIDDLRESPAMEIAELIAQWHSGETLVVEPNIHQLPKKLTGLCTLAQLDEALATADVLVMLVDHSQFKVINGDNVHQQYVVDAKGVWR

SEQ ID NO: 19 - protein, 355 aa, Excherichia coli
MRKILITGGAGFIGSALVRYIINETSDAVVVVDKLTYAGNLMSLAPVAQSERFAFEKVDICDRAELARVFTEHQPDCVMHLAAESHVDRSIDGPAAFIETNIVGTYTLLEAARAYWNALTEDKKSAFRFHHISTDEVYGDLHSTDDFFTETTPYAPSSPYSASKASSDHLVRAWLRTYGLPTLITNCSNNYGPYHFPEKLIPLMILNALAGKSLPVYGNGQQIRDWLYVEDHARALYCVATTGKVGETYNIGGHNERKNLDVVETICELLEELAPNKPHGVAHYRDLITFVADRPGHDLRYAIDASKIARELGWLPQETFESGMRKTVQWYLANESWWKQVQDGSYQGERLGLKG

SEQ ID NO: 20 - protein, 293 aa, Excherichia coli
MKGIILAGGSGTRLHPITRGVSKQLLPIYDKPMIYYPLSVLMLAGIREILIITTPEDKGYFQRLLGDGSEFGIQLEYAEQPSPDGLAQAFIIGETFLNGEPSCLVLGDNIFFGQGFSPKLRHVAARTEGATVFGYQVMDPERFGVVEFDDNFRAISLEEKPKQPKSNWAVTGLYFYDSKVVEYAKQVKPSERGELEITSINQMYLEAGNLTVELLGRGFAWLDTGTHDSLIEASTFVQTVEKRQGFKIACLEEIAWRNGWLDDEGVKRAASSLAKTGYGQYLLELLRARPRQY

SEQ ID NO: 21 - protein, 224 aa, Excherichia coli
MPVRASIEPLTWENAFFGVNSAIVRITSEAPLLTPDALAPWSRVQAKIAASNTGELDALQQLGFSLVEGEVDLALPVNNASDSGAVVAQETDIPALRQLASAAFAQSRFRAPWYAPDASSRFYAQWIENAVRGTFDHQCLILRAASGDIRGYVSLRELNATDARIGLLAGRGAGAELMQTALNWAYARGKTTLRVATQMGNTAALKRYIQSGANVESTAYWLYR

SEQ ID NO: 22 - protein, 376 aa, Excherichia coli
MIPFNAPPVVGTELDYMQSAMGSGKLCGDGGFTRRCQQWLEQRFGSAKVLLTPSCTASLEMAALLLDIQPGDEVIMPSYTFVSTANAFVLRGAKIVFVDVRPDTMNIDETLIEAAITDKTRVIVPVHYAGVACEMDTIMALAKKHNLFVVEDAAQGVMSTYKGRALGTIGHIGCFSFHETKNYTAGGEGGATLINDKALIERAEIIREKGTNRSQFFRGQVDKYTWRDIGSSYLMSDLQAAYLWAQLEAADRINQQRLALWQNYYDALAPLAKAGRIELPSIPDGCVQNAHMFYIKLRDIDDRSALINFLKEAEIMAVFHYIPLHGCPAGEHFGEFHGEDRYTTKESERLLRLPLFYNLSPVNQRTVIATLLNYFS

SEQ ID NO: 23 - protein, 416 aa, Excherichia coli
MSLAKASLWTAASTLVKIGAGLLVGKLLAVSFGPAGLGLAANFRQLITVLGVLAGAGIFNGVTKYVAQYHDNPQQLRRVVGTSSAMVLGFSTLMALVFVLAAAPISQGLFGNTDYQGLVRLVALVQMGIAWGNLLLALMKGFRDAAGNALSLIVGSLIGVLAYYVSYRLGGYEGALLGLALIPALVVIPAAIMLIKRGVIPLSYLKPSWDNGLAGQLSKFTLMALITSVTLPVAYIMMRKLLAAQYSWDEVGIWQGVSSISDAYLQFITASFSVYLLPTLSRLTEKRDITREVVKSLKFVLPAVAAASFTVWLLRDFAIWLLLSNKFTAMRDLFAWQLVGDVLKVGAYVFGYLVIAKASLRFYILAEVSQFTLLMVFAHWLIPAHGALGAAQAYMATYIVYFSLCCGVFLLWRRRA

SEQ ID NO: 24 - protein, 359 aa, Excherichia coli
MTVLIHVLGSDIPHHNRTVLRFFNDALAATSEHAREFMVVGKDDGLSDSCPALSVQFFPGKKSLAEAVIAKAKANRQQRFFFHGQFNPTLWLALLSGGIKPSQFFWHIWGADLYELSSGLRYKLFYPLRRLAQKRVGCVFATRGDLSFFAKTHPKVRGELLFFPTRMDPSLNTMANDRQREGKMTILVGNSGDRSNEHIAALRAVHQQFGDTVKVVVPMGYPPNNEAYIEEVRQAGLELFSEENLQILSEKLEFDAYLALLRQCDLGYFIFARQQGIGTLCLLIQAGIPCVLNRENPFWQDMTEQHLPVLFTTDDLNEDIVREAQRQLASVDKNTIAFFSPNYLQGWQRALAIAAREVA

SEQ ID NO: 25 - protein, 450 aa, Excherichia coli
MSLLQFSGLFVVWLLCTLFIATLTWFEFRRVRFNFNVFFSLLFLLTFFFGFPLTSVLVFRFDVGVAPPEILLQALLSAGCFYAVYYVTYKTRLRKRVADVPRRPLFTMNRVETNLTWVILMGIALVSVGIFFMHNGFLLFRLNSYSQIFSSEVSGVALKRFFYFFIPAMLVVYFLRQDSKAWLFFLVSTVAFGLLTYMIVGGTRANIIIAFAIFLFIGIIRGWISLWMLAAAGVLGIVGMFWLALKRYGMNVSGDEAFYTFLYLTRDTFSPWENLALLLQNYDNIDFQGLAPIVRDFYVFIPSWLWPGRPSMVLNSANYFTWEVLNNHSGLAISPTLIGSLVVMGGALFIPLGAIVVGLIIKWFDWLYELGNREPNRYKAAILHSFCFGAIFNMIVLAREGLDSFVSRVVFFIVVFGACLMIAKLLYWLFESAGLIHKRTKSSLRTQVEG

SEQ ID NO: 26 - protein, 246 aa, Excherichia coli
MNNNTTAPTYTLRGLQLIGWRDMQHALDYLFADGQLKQGTLVAINAEKMLTIEDNAEVRELINAAEFKYADGISVVRSVRKKYPQAQVSRVAGADLWEELMARAGKEGTPVFLVGGKPEVLAQTEAKLRNQWNVNIVGSQDGYFKPEQRQALFERIHASGAQIVTVAMGSPKQEIIMRDCRLVHPDALYMGVGGTYDVFTGHVKRAPKIWQTLGLEWLYRLLSQPSRIKRQLRLLRYLRWHYTGNL

SEQ ID NO: 27 - protein, 264 aa, Excherichia coli
MVYIIIVSHGHEDYIKKLLENLNADDEHYKIIVRDNKDSLLLKQICQHYAGLDYISGGVYGFGHNNNIAVAYVKEKYRPADDDYILFLNPDIIMKHDDLLTYIKYVESKRYAFSTLCLFRDEAKSLHDYSVRKFPVLSDFIVSFMLGINKTKIPKESIYSDTVVDWCAGSFMLVRFSDFVRVNGFDQGYFMYCEDIDLCLRLSLAGVRLHYVPAFHAIHYAHHDNRSFFSKAFRWHLKSTFRYLARKRILSNRNFDRISSVFHP

SEQ ID NO: 28 - protein, 372 aa, Excherichia coli
MGKSIVVVSAVNFTTGGPFTILKKFLAATNNKENVSFIALVHSAKELKESYPWVKFIEFPEVKGSWLKRLHFEYVVCKKLSKELNATHWICLHDITANVVTKKRYVYCHNPAPFYKGILFREILMEPSFFLFKMLYGLIYKINIKKNTAVFVQQFWMKEKFIKKYSINNIIVSRPEIKLSDKSQLTDDDSQFKNNPSELTIFYPAVPRVFKNYELIISAARKLKEQSNIKFLLTISGTENAYAKYIISLAEGLDNVHFLGYLDKEKIDHCYNISDIVCFPSRLETWGLPLSEAKERGKWVLASDFPFTRETLGSYEKKAFFDSNNDDMLVKLIIDFKKGNLKKDISDANFIYRNENVLVGFDELVNFITEEH

SEQ ID NO: 29 - protein, 196 aa, Excherichia coli
MILKLAKRYGLCGFIRLVRDVLLTRVFYRNCRIIRFPCYIRNDGSINFGENFTSGVGLRLDAFGRGVIFFSDNVQVNDYVHIASIESVTIGRDTLIASKVFITDHNHGSFKHSDPMSSPNIPPDMRTLESSAVVIGQRVWLGENVTVLPGTIIGNGVVVGANSVVRGSIPENTVIAGVPAKIIKKYNHETKLWEKA

SEQ ID NO: 30 - protein, 330 aa, Excherichia coli
MYFLNDLNFSRRDAGFKARKDALDIASDYENISVVNIPLWGGVVQRIISSVKLSTFLCGLENKDVLIFNFPMAKPFWHILSFFHRLLKFRIVPLIHDIDELRGGGGSDSVRLATCDMVISHNPQMTKYLSKYMSQDKIKDIKIFDYLVSSDVEHRDVTDKQRGVIYAGNLSRHKCSFIYTEGCDFTLFGVNYENKDNPKYLGSFDAQSPEKINLPGMQFGLIWDGDSVETCSGAFGDYLKFNNPHKTSLYLSMELPVFIWDKAALADFIVDNRIGYAVGSIKEMQEIVDSMTIETYKQISENTKIISQKIRTGSYFRDVLEEVIDDLKTR

SEQ ID NO: 31 - protein, 388 aa, Excherichia coli
MIYLVISVFLITAFICLYLKKDIFYPAVCVNIIFALVLLGYEITSDIYAFQLNDATLIFLLCNVLTFTLSCLLTESVLDLNIRKVNNAIYSIPSKKVHNVGLLVISFSMIYICMRLSNYQFGTSLLSYMNLIRDADVEDTSRNFSAYMQPIILTTFALFIWSKKFTNTKVSKTFTLLVFIVFIFAIILNTGKQIVFMVIISYAFIVGVNRVKHYVYLITAVGVLFSLYMLFLRGLPGGMAYYLSMYLVSPIIAFQEFYFQQVSNSASSHVFWFFERLMGLLTGGVSMSLHKEFVWVGLPTNVYTAFSDYVYISAELSYLMMVIHGCISGVLWRLSRNYISVKIFYSYFIYTFSFIFYHESFMTNISSWIQITLCIIVFSQFLKAQKIK

SEQ ID NO: 32 - protein, 367 aa, Excherichia coli [sequence omitted]

SEQ ID NO: 33 - protein, 415 aa, Excherichia coli
MNTNKLSLRRNVIYLAVVQGSNYLLPLLTFPYLVRTLGPENFGIFGFCQATMLYMIMFVEYGFNLTATQSIAKAADSKDKVTSIFWAVIFSKIVLIVITLIFLTSMTLLVPEYNKHAVIIWSFVPALVGNLIYPIWLFQGKEKMKWLTLSSILSRLAIIPLTFIFVNTKSDIAIAGFIQSSANLVAGIIALAIVVHEGWIGKVTLSLHNVRRSLADGFHVFISTSAISLYSTGIVIILGFISGPTSVGNFNAANTIRNALQGLLNPITQAIYPRISSTLVLNRVKGVILIKKSLTCLSLIGGAFSLILLLGASILVKISIGPGYDNAVIVLMIISPLPFLISLSNVYGIQVMLTHNYKKEFSKILIAAGLLSLLLIFPLTTLFKEIGAAITLLATECLVTSLMLMFVRNNKLLVC

SEQ ID NO: 34 - protein, 185 aa, Excherichia coli
MNVIRTEIEDVLILEPRVFGDDRGFFYESFNQSAFEHILGYPVSFVQDNHSRSSKNVLRGLHFQRGEYAQDKLVRCTHGAVFDVAVDIRPNSVSFGKWVGVLLSADNKQQLWIPKGFAHGFLVLSDIAEFQYKTTNYYHPESDCGICWNDERIAIDWPQTSGLILSPKDERLFTLDELIRLKLIA

SEQ ID NO: 35 - protein, 293 aa, Excherichia coli
MKMRKGIILAGGSGTRLYPVTMAVSKQLLPIYDKPMIYYPLSTLMLAGIRDILIISTPQDTPRFQQLLGDGSQWGLNLQYKVQPSPDGLAQAFIIGEEFIGGDDCALVLGDNIFYGHDLPKLMEAAVNKESGATVFAYHVNDPERYGVVEFDKNGTAISLEEKPLEPKSNYAVTGLYFYDNDVVQMAKNLKPSARGELEITDINRIYLEQGRLSVAMMGRGYAWLDTGTHQSLIEASNFIATIEERQGLKVSCPEEIAFRKGFIDVEQVRKLAVPLIKNNYGQYLYKMTKDSN

SEQ ID NO: 36 - protein, 299 aa, Excherichia coli
MNILLFGKTGQVGWELQRALAPLGNLIAFDVHSTDYCGDFSNPEGVAETVRSIRPDIIVNAAAHTAVDKAESEPEFAQLINATSVEAIAKAANEVGAWVIHYSTDYVFPGNGDMPWLETDATAPLNVYGETKLAGEKALQEYCAKHLIFRTSWVYAGKGNNFAKTMLRLAKEREELAVINDQFGAPTGAELLADCTAHAIRVALNKPDVAGLYHLVASGTTTWYDYAALVFEEARKAGIPLALNKLNAVPTTAYPTPARRPHNSRLNTEKFQQNFALVLPDWQVGVKRMLNELFTTTAI

SEQ ID NO: 37 - protein, 361 aa, Excherichia coli
MKILVTGGAGFIGSAVVRHIINNTQDSVVNVDKLTYAGNRESLADVSDSERYVFEHADICDAPAMARIFAQHQPDAVMHLAAESHVDRSITGPAAFIETNIVGTYVLLEAARNYWSALDSDKKNSFRFHHISTDEVYGDLPHPDEVNNTEELPLFTETTAYAPSSPYSASKASSDHLVRAWKRTYGLPTIVTNCSNNYGPYHFPEKLIPLVILNALEGKALPIYGKGDQIRDWLYVEDHARALYTVVTEGKAGETYNIGGHNEKKNIDVVLTICDLLDEIVPKEKSYREQITYVADRPGHDRRYAIDAEKIGRALGWKPQETFESGIRKTVEWYLSNTKWVDNVKSGAYQSWIEQNYEGRQ

SEQ ID NO: 38 - protein, 297 aa, Excherichia coli
MTNLKAVIPVAGLGMHMLPATKAIPKEMLPIVDKPMIQYIVDEIVAAGIKEILLVTHASKNAVENHFDTSYELESLLEQRVKRQLLAEVQSICPPGVTIMNVRQGEPLGLGHSILCARPAIGDNPFVVVLPDVVIDDASADPLRYNLAAMIARFNETGRSQVLAKRMPGDLSEYSVIQTKEPLDREGKVSRIVEFIEKPDQPQTLDSDIMAVGRYVLSADIWPELERTQPGAWGRIQLTDAIAELAKKQSVDAMLMTGDSYDCGKKMGYMQAFVKYGLRNLKEGAKFRKGIEKLLSE

SEQ ID NO: 39 - protein, 464 aa, Excherichia coli
MPFKKLSRRTFLTASSALAFLHTPFARALPARQSVNINDYNPHDWIASFKQAFSEGQTVVVPAGLVCDNINTGIFIPPGKTLHILGSLRGNGRGRFVLQDGSQVTGEDGGSMHNITLDVRGSDCTIKGLTMSGFGPVTQIYIGGKNKRVMRNLIIDNLTVSHANYAILRQGFHNQIIGANITNCKFSDLQGDAIEWNVAINDRDILISDHVIERINCTNGKINWGIGIGLAGSTYDNNYPEDQAVKNFVVANITGSDCRQLIHVENGKHFVIRNIKARNITPDFSKKAGIDNATVAIYGCDNFVIDNIEMINSAGMLIGYGVIKGKYLSIPQNFRVNDIQLDNTHLAYKLRGIQISAGNAVSFVALTNIEMKRASLELHNKPQHFFMRNINVMQESSVGPALSMNFDMRKDVRGVFMAKKETLLSLANVHAVNEKGQSSVDIDRINHHIVNVEKINFRLPELRE

SEQ ID NO: 40 - protein, 406 aa, Excherichia coli
MKVGFFLLKFPLSSETFVLNQITAFIDMGFEVEILALQKGDTQNTHAAWTKYNLAARTRWLQDEPTGKVAKLRHRASQTLRGIHRKNTWQALNLKRYGAESRNLILSAICGQVATPFRADVFIAHFGPAGVTAAKLRELGVIRGKIATIFHGIDISSREVLNHYTPEYQQLFRRGDLMLPISDLWAGRLQKMGCPREKIAVSRMGVDMTRFSPRPVKAPATPLEIISVARLTEKKGLHVAIEACRQLKEQGVAFRYRILGIGPWERRLRTLIEQYQLEDVVEMPGFKPSHEVKAMLDDADVFLLPSVTGADGDMEGIPVALMEAMAVGIPVVSTLHSGIPELVEADKSGWLVPENDARALAQRLAAFSQLDTDELAPVVKRAREKVEHDFNQQVINRELASLLQAL

SEQ ID NO: 41 - protein, 426 aa, Excherichia coli [sequence omitted]

SEQ ID NO: 42 - protein, 492 aa, Excherichia coli
MSLREKTISGAKWSAIATVIIIGLGLVQMTVLARIIDNHQFGLLTVSLVIIALADTLSDFGIANSIIQRKEISHLELTTLYWLNVGLGIVVCVAVFLLSDLIGDVLNNPDLAPLIKTLSLAFVVIPHGQQFRALMQKELEFNKIGMIETSAVLAGFTCTVVSAHFWPLAMTAILGYLVNSAVRTLLFGYFGRKIYRPGLHFSLASVAPNLRFGAWLTADSIINYLNTNLSTLVLARILGAGVAGGYNLAYNVAVVPPMKLNPIITRVLFPAFAKIQDDTEKLRVNFYKLLSVVGIINFPALLGLMVVSNNFVPLVFGEKWNSIIPVLQLLCVVGLLRSVGNPIGSLLMAKARVDISFKFNVFKTFLFIPAIVIGGQMAGAIGVTLGFLLVQIINTILSYFVMIKPVLGSSYRQYILSLWLPFYLSLPTLVVSYALGIVLKGQLALGMLLAVQIATGVLAFVVMIVLSRHPLVVEVKRQFCRSEKMKMLLRAG

SEQ ID NO: 43 - protein, 464 aa, Excherichia coli
MTNLKKRERAKTNASLISMVQRFSDITIMFAGLWLVCEVSGLSFLYMHLLVALITLVVFQMLGGITDFYRSWRGVRAATEFALLLQNWTLSVIFSAGLVAFNNDFDTQLKIWLAWYALTSIGLVVCRSCIRIGAGWLRNHGYNKRMVAVAGDLAAGQMLMESFRNQPWLGFEVVGVYHDPKPGGVSNDWAGNLQQLVEDAKAGKIHNVYIAMQMCDGARVKKLVHQLADTTCSVLLIPDVFTFNILHSRLEEMNGVPVVPLYDTPLSGVNRLLKRAEDIVLATLILLLISPVLCCIALAVKLSSPGPVIFRQTRYGMDGKPIKVWKFRSMKVMENDKVVTQATQNDPRVTKVGNFLRRTSLDELPQFINVLTGGMSIVGPRPHAVAHNEQYRQLIEGYMLRHKVKPGITGWAQINGWRGETDTLEKMEKRVEFDLEYIREWSVWFDIKIVFLTVFKGFVNKAAY

SEQ ID NO: 44 - protein, 456 aa, Excherichia coli
MKKLTCFKAYDIRGKLGEELNEDIAWRIGRAYGEFLKPKTIVLGGDVRLTSETLKLALAKGLQDAGVDVLDIGMSGTEEIYFATFHLGVDGGIEVTASHNPMDYNGMKLVREGARPISGDTGLRDVQRLAEANDFPPVDETKRGRYQQINLRDAYVDHLFGYINVKNLTPLKLVINSGNGAAGPVVDAIEARFKALGAPVELIKVHNTPDGNFPNGIPNPLLPECRDDTRNAVIKHGADMGIAFDGDFDRCFLFDEKGQFIEGYYIVGLLAEAFLEKNPGAKIIHDPRLSWNTVDVVTAAGGTPVMSKTGHAFIKERMRKEDAIYGGEMSAHHYFRDFAYCDSGMIPWLLVAELVCLKDKTLGELVRDRMAAFPASGEINSKLAQPVEAINRVEQHFSREALAVDRTDGISMTFADWRFNLRTSNTEPVVRLNVESRGDVPLMEARTRTLLTLLNE

SEQ ID NO: 45 - protein, 478 aa, Excherichia coli
MAQSKLYPVVMAGGSGSRLWPLSRVLYPKQFLCLKGDLTMLQTTICRLNGVECESPVVICNEQHRFIVAEQLRQLNKLTENIILEPAGRNTAPAIALAALAAKRHSPESDPLMLVLAADHVIADEDAFRAAVRNAMPYAEAGKLVTFGIVPDLPETGYGYIRRGEVSAGEQDMVAFEVAQFVEKPNLETAQAYVASGEYYWNSGMFLFRAGRYLEELKKYRPDILDACEKAMSAVDPDLNFIRVDEEAFLACPEESVDYAVMERTADAVVVPMDAGWSDVGSWSSLWEISAHTAEGNVCHGDVINHKTENSYVYAESGLVTTVGVKDLVVVQTKDAVLIADRNAVQDVKKVVEQIKADGRHEHRVHREVYRPWGKYDSIDAGDRYQVKRITVKPGEGLSVQMHHHRAEHWVVVAGTAKVTIDGDIKLLGENESIYIPLGATHCLENPGKIPLDLIEVRSGSYLEEDDVVRFADRYGRV

SEQ ID NO: 46 - protein, 407 aa, Excherichia coli
MKILVYGINYSPELTGIGKYTGEMVEWLAAQGHEVRVITAPPYYPQWQVGENYSAWRYKREEGAATVWRCPLYVPKQPSTLKRLLHLGSFAVSSFFPLMAQRRWKPDRIIGVVPTLFCAPGMRLLAKLSGARTVLHIQDYEVDAMLGLGLAGKGKGGKVAQLATAFERSGLHNVDNVSTISRSMMNKAIEKGVAAENVIFFPNWSEIARFQHVADADVDALRNQLDLPDNKKIILYSGNIGEKQGLENVIEAADRLRDEPLIFAIVGQGGGKARLEKMAQQRGLRNMQFFPLQSYDALPALLKMGDCHLVVQKRGAADAVLPSKLTNILAVGGNAVITAEAYTELGQLCETFPGIAVCVEPESVEALVAGIRQALLLPKHNTVAREYAERTLDKENVLRQFINDIRG

SEQ ID NO: 47 - protein, 159 aa, Excherichia coli
MFLRQEDFATVVRSTPLVSLDFIVENSRGEFLLGKRTNRPAQGYWFVPGGRVQKDETLEAAFERLTMAELGLRLPITAGQFYGVWQHFYDDNFSGTDFTTHYVVLGFRFRVSEEELLLPDEQHDDYRWLTSDALLASDNVHANSRAYFLAEKRTGVPGL

SEQ ID NO: 48 - protein, 321 aa, Excherichia coli
MSKQRVFIAGHRGMVGSAIRRQLEQRGDVELVLRTRDELNLLDSRAVHDFFASERIDQVYLAAAKVGGIVANNTYPADFIYQNMMIESNIIHAAHQNDVNKLLFLGSSCIYPKLAKQPMAESELLQGTLEPTNEPYAIAKIAGIKLCESYNRQYGRDYRSVMPTNLYGPHDNFHPSNSHVIPALLRRFHEATAQNAPDVVVWGSGTPMREFLHVDDMAAASIHVMELAHEVWLENTQPMLSHINVGTGVDCTIRELAQTIAKVVGYKGRVVFDASKPDGTPRKLLDVTRLHQLGWYHEISLEAGLASTYQWFLENQDRFRG

SEQ ID NO: 49 - protein, 373 aa, Excherichia coli [sequence omitted]

SEQ ID NO: 50 - protein, 182 aa, Excherichia coli
MQDLSGFSVPKGFRGGNAIKVQLWWAVQATIFAWSPQVLYRWRAFLLRLFGAKIGKNVVIRPSVKITYPWKLTLGDYAWVGDDVNLYTLGEITIGAHSVISQKSYLCTGSHDHASQHFTINATPIVIGEKCWLATDVFVAPGVTIGDGTVVGARSSVFKSLPANVVCRGNPAVVIRERVETE

SEQ ID NO: 51 - protein, 248 aa, Excherichia coli
MLLSIITVAFRNLEGIVKTHASLAHLAQVEDISFEWIVVDGGSNDGTREYLENLNGIFNLRFVSEPDNGIYDAMNKGIAMAQGKFALFLNSGDIFHQNAANFVRKLKMQKDNVMITGDALLDFGDGHKIKRSAKPGWYIYHSLPASHQAIFFPVSGLKKWRYDLEYKVSSDYALAAKMYKAGYAFKKLNGLVSEFSMGGVSTTNNMELCADAKKVQRQILHVPGFWAELSWHLRQRTTSKTKALYNKV

SEQ ID NO: 52 - protein, 405 aa, Excherichia coli
MSTSIRICSYLLLPLIYLLVNVKIAQLGESFPITIVTFLPVLLLLFLERISVKKLMIALGIGAGLTAFNYLFGQSLDASKYVTSTMLFVYIVIIIGMVWSIRFKTISPHNHRKILRFFYLVVGLVVALAAVEMAQIILTGGSSIMESISKYLIYSNSYVLNFIKFGGKRTTALYFEPAFFALALISIWLSIKQFGIKTPKTDAMILAGIILSGSFSGVMTFILFYLLEWAFQYLNKEAIKKKLPLALISLAVFLVGVVIAFPYISTRLGDLGTEGSSSYYRIVGPLVMVGYSLTHIDGVVRFGSLYEYVASFGIFNGADVGKTIDNGLYLLIIYFSWFAVFLSLWYMGKVIKMMINAFGDNRNFRVQLYLFTPVSLFFTGSIFSPEYAFLIVCPFILRKALNITR

SEQ ID NO: 53 - protein, 405 aa, Excherichia coli
MNILQFNVRLAEGGAAGVALDLHQRALQQGLASHFVYGYGKGGKESVSHQNYPQVIKHTPRMTAMANIALFRLFNRDLFGNFNELYRTITRTAGPVVLHFHVLHSYWLNLKSVVRFCEKVKNHKPDVTLVWTLHDHWSVTGRCAFTDGCEGWKTGCQKCPTLNNYPPVKIDRAHQLVAGKRQLFREMLALGCQFISPSQHVADAFNSLYGPGRCRIINNGIDMATEAILADLPPVRETQGKPKIAVVAHDLRYDGKTNQQLVREMMALGDKIELHTFGKFSPFTAGNVVNHGFETDKRKLMSALNQMDALVFSSRVDNYPLILCEALSIGVPVIATHSDAAREVLQKSGGKTVSEEEVLQLVQLSKPEIAQAIFGTTLAEFSQRSRAAYSGQQMLEEYVNFYQNL

SEQ ID NO: 54 - protein, 162 aa, Excherichia coli
MLEDLRANSWSLRPCCMVLAYRVAHFCSVWRKKNVLNNLWAAPLLVLYRIITECFFGYEIQAAATIGRRFTIHHGYAVVINKNVVAGDDFTIRHGVTIGNRGADNMACPHIGNGVELGANVIILGDITLGNNVTVGAGSVVLDSVPDNALVVGEKARVKVIK

SEQ ID NO: 55 - protein, 279 aa, Excherichia coli
MKNNPLISIYMPTWNRQQLAIRAIKSVLRQDYSNWEMIIVDDCSTSWEQLQQYVTALNDPRITYIHNDINSGACAVRNQAIMLAQGEYITGIDDDDEWTPNRLSVFLAHKQQLVTHAFLYANDYVCQGEVYSQPASLPLYPKSPYSRRLFYKRNIIGNQVFTWAWRFKECLFDTELKAAQDYDIFLRMVVEYGEPWKVEEATQILHINHGEMQITSSPKKFSGYFHFYRKHKDKFDRASKKYQLFTLYQIRNKRMTWRTLLTLLSVRNGKRLADGIRGR

SEQ ID NO: 56 - protein, 720 aa, Excherichia coli
MTEKVKQHAAPVTGSDEIDIGRLVGTVIEARWWVIGITTVFALCAVVYTFFATPIYSADALVQIEQNSGNSLVQDIGSALANKPPASDAEIQLIRSRLVLGKTVDDLDLDIAVSKNTFPIFGAGWDRLMGRQNETVKVTTFNRPKEMADQVFTLNVLDNKNYTLSSDGGFSARGQAGQMLKKEGVTLMVEAIHASPGSEFTVTKYSTLGMINQLQNSLTVTENGKDAGVLSLTYTGEDREQIRDILNSIARNYQEQNIERKSAEASKSLAFLAQQLPEVRSRLDVAENKLNAFRQDKDSVDLPLEAKAVLDSMVNIDAQLNELTFKEAEISKLYTKVHPAYRTLLEKRQALEDEKAKLNGRVTAMPKTQQEIVRLTRDVESGQQVYMQLLNKEQELKITEASTVGDVRIVDPAITQPGVLKPKKGLIILGAIILGLMLSIVGVLLRSLFNRGIESPQVLEEHGISVYASIPLSEWQKARDSVKTIKGIKRYKQSQLLAVGNPTDLAIEAIRSLRTSLHFAMMQAQNNVLMMTGVSPSIGKTFVCANLAAVISQTNKRVLLIDCDMRKGYTHELLGTNNVNGLSEILIGQGDITTAAKPTSIAKFDLIPRGQVPPNPSELLMSERFAELVNWASKNYDLVLIDTPPILAVTDAAIVGRHVGTTLMVARYAVNTLKEVETSLSRFEQNGIPVKGVILNSIFRRASAYQDYGYYEYEYKSDAK

SEQ ID NO: 57 - protein, 147 aa, Excherichia coli
MFNNILVVCVGNICRSPTAERLLQRYHPELKVESAGLGALVGKGADPTAISVAAEHQLSLEGHCARQISRRLCRNYDLILTMEKRHIERLCEMAPEMRGKVMLFGHWDNECEIPDPYRKSRETFAAVYTLLERSARQWAQALNAEQV

SEQ ID NO: 58 - protein, 379 aa, Excherichia coli
MMKSKMKLMPLLVSVTLISGCTVLPGSNMSTMGKDVIKQQDADFDLDKMVNVYPLTPRLIDQLRPRPNVARPNMTLESEIANYQYRVGPGDVLNVTVWDHPELTTPAGQYRSSSDTGNWVQPDGTMFYPYIGKVHVVGKTLAEIRSDITGRLATYIADPQVDVNIAAFRSQKAYISGQVNKSGQQAITNVPLTILDAINAAGGLTDTADWRNVVLTHNGREERISLQALMQNGDLNQNRLLYPGDILYVPRNDDLKVFVMGEVKKQSTLKMDFSGMTLTEALGNAEGIDMTTSNASGIFVIRPLKGEGGRNGKIANIYQLDMSDATSLVMATEFRLQPYDVVYVTTAPVSRWNRLINQLLPTISGVRYMTDTASDIHNW